SEQUENCE LISTING (12 sequences)

SEQ ID NO: 1 - protein, 7 aa, synthetic construct
GYTFDRY

SEQ ID NO: 2 - protein, 6 aa, synthetic construct
LPGSGD

SEQ ID NO: 3 - protein, 11 aa, synthetic construct
MYYDYDQGFDL

SEQ ID NO: 4 - protein, 11 aa, synthetic construct
KFSQDIDRFLS

SEQ ID NO: 5 - protein, 7 aa, unidentified
  source 1..7: note = Murine
RVKRLVD

SEQ ID NO: 6 - protein, 9 aa, synthetic construct
VQYDEFPYG

SEQ ID NO: 7 - protein, 120 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFDLWGQGTLVTVSS

SEQ ID NO: 8 - protein, 107 aa, synthetic construct
DIQMTQSPSSLSASVGDRVTITCKFSQDIDRFLSWFQQKPGKAPKSLIYRVKRLVDGVPSRFSGSGSGTDFTLTISSLQPEDFATYYCVQYDEFPYGFGGGTKVEIK

SEQ ID NO: 9 - protein, 467 aa, synthetic construct [sequence omitted]

SEQ ID NO: 10 - protein, 447 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, synthetic construct
DIQMTQSPSSLSASVGDRVTITCKFSQDIDRFLSWFQQKPGKAPKSLIYRVKRLVDGVPSRFSGSGSGTDFTLTISSLQPEDFATYYCVQYDEFPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 100 aa, synthetic construct
DIQMTQSPSSLSASVGDRVTITCKFSQDIDRFLSWFQQKPGKAPKSLIYRVKRLVDGVPSRFSGSGSGTDFTLTISSLQPEDFATYYCVQYDEFPYGFGG

(19) United States
(12) Patent Application Publication
(10) Pub. No.: US 2026/0103511 A1
(43) Pub. Date: Apr. 16, 2026

(54) Liquid Pharmaceutical Formulations for an Anti-IL-1beta Antibody

(71) Applicant: Avalo Therapeutics, Inc., Rockville, MD (US)

(72) Inventors: Virginie Le Brun, Basel (CH); Dino C. Miano, Rockville, MD (US)

(21) Appl. No.: 19/359,490

(22) Filed: Oct. 15, 2025

Related U.S. Application Data
(60) Provisional application No. 63/707,988, filed on Oct. 16, 2024.

(51) Int. Cl. C07K 16/24; A61K 9/00; A61K 39/00; A61K 39/395; A61K 47/02; A61K 47/18; A61K 47/20; A61K 47/22; A61K 47/26

(52) U.S. Cl. CPC C07K 16/245; A61K 9/0019; A61K 39/39591; A61K 47/02; A61K 47/183; A61K 47/20; A61K 47/22; A61K 47/26; A61K 2039/505; C07K 2317/52; C07K 2317/55; C07K 2317/565

(57) ABSTRACT

The present disclosure relates to improved liquid pharmaceutical formulations for an antibody that binds to interleukin-1-β (IL-1β) that may be stored for long periods of time and that may be administered by both intravenous infusion and subcutaneous injection.

### CROSS-REFERENCE TO RELATED APPLICATION

This patent application claims the benefit of priority from U.S. Provisional Application No. 63/707,988, filed Oct. 16, 2024, the contents of which are incorporated herein by reference in their entirety for all purposes.

### SEQUENCE LISTING

The instant application contains a Sequence Listing that has been submitted electronically in XML format and is hereby incorporated by reference in its entirety. Said XML copy, created on Oct. 14, 2025, is named “01118-0071-00PCT-ST26.xml” and is 13,032 bytes in size.

### FIELD OF THE INVENTION

The invention relates to improved liquid pharmaceutical formulations for an antibody that binds to interleukin-1-beta (IL-1β) that may be stored for long periods of time and that may be administered by both intravenous infusion and subcutaneous injection.

### BACKGROUND OF THE INVENTION

The present invention relates to liquid pharmaceutical formulations of an antibody that binds to interleukin-1-β (IL-1β).

IL-1β is expressed in a wide range of tissues and cells, including blood monocytes, lymphoid and non-lymphoid macrophages, and dendritic cells. IL-1β exerts strong proinflammatory activities at very low concentrations. Inactive IL-1β precursors accumulate in the cytosol until processed by NLRP3 inflammasome and caspase-1 into its active cytokine form (IL-1β). Inflammasome activation leads to rapid increases in active IL-1β.

IL-1β antibodies have been disclosed previously. Anti-IL-1β antibodies have been proposed or evaluated for the treatment of autoimmune disorders and other conditions associated with inflammation, including rheumatoid arthritis, osteoarthritis, and neuroinflammation, as disclosed in WO 2004/067568 and U.S. Pat. Nos. 7,541,033 and 7,714,200.

WO 2004/067568 and U.S. Pat. Nos. 7,541,033 and 7,714,200 provide general guidance on formulating anti-IL-1β antibodies at concentrations suitable for intravenous infusion, but not for subcutaneous injections. For example, WO 2004/067568 discloses that anti-IL-1β antibodies may be formulated for intravenous infusion, preferably, at concentrations of 1 to about 100 mg/mL or 10 to about 50 mg/mL, may comprise a buffer, preferably, a citrate or phosphate buffer. may have a pH between about 4 to 8, preferably, between about 5 and 7.5; may include a salt such as NaCl, may include a detergent to prevent aggregation and aid in maintaining stability. WO 2004/067568 additionally discloses that a typical composition for intravenous infusion of the anti-IL-1β antibodies could have a volume as much as 250 ml of fluid.

WO 2004/067568 and U.S. Pat. Nos. 7,541,033 and 7,714,200 do not disclose any specific formulations for anti-IL-1β antibodies or provide any specific guidance for formulating such antibodies for subcutaneous injection.

There remains a need for improved options for formulating anti-IL-1β antibodies, including a need for formulations with prolonged stability that are suitable for administration by both intravenous infusion and subcutaneous injection.

### SUMMARY OF THE INVENTION

The present disclosure includes, for example, novel liquid pharmaceutical formulations of anti-IL-1β antibodies that exhibit long-term stability and are suitable for both intravenous infusion and subcutaneous injection. The invention is exemplified by the non-limiting embodiments below.

Embodiment 1. A liquid pharmaceutical formulation comprising: a concentration of an anti-IL-1β antibody of 100 mg/mL to 200 mg/mL; at least one buffering agent; at least one colloidal stabilizer; at least one antioxidant; at least one saccharide; and at least one surfactant, wherein the anti-IL-1β antibody comprises:
(i) a heavy chain CDR1 (HCDR1) comprising the amino acid sequence of SEQ ID NO: 1; a heavy chain CDR2 (HCDR2) comprising the amino acid sequence of SEQ ID NO: 2; a heavy chain CDR3 (HCDR3) comprising the amino acid sequence of SEQ ID NO: 3; a light chain CDR1 (LCDR1) comprising the amino acid sequence of SEQ ID NO: 4; a light chain CDR2 (LCDR2) comprising the amino acid sequence of SEQ ID NO: 5; and a light chain CDR3 (LCDR3) comprising the amino acid sequence of SEQ ID NO: 6; or
(ii) a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7; a VL comprising an acid sequence chosen from: SEQ ID NO: 8 and SEQ ID NO: 12; a HC comprising an amino acid sequence chosen from: SEQ ID NO: 9 and SEQ ID NO: 10; and a LC comprising the amino acid sequence of SEQ ID NO: 11;
(iii) variable heavy chain region (VH) comprising the amino acid sequence of SEQ ID NO: 7; or
(iv) a variable light chain region (VL) comprising an amino acid sequence chosen from: SEQ ID NO: 8 and SEQ ID NO: 12;
(v) a VH comprising the amino acid sequence of SEQ ID NO: 7 and a VL comprising an amino acid sequence chosen from: SEQ ID NO: 8 and SEQ ID NO: 12;
(vi) a heavy chain (HC) comprising an amino acid sequence having at least 85%, 90%, 91%, 92%, 93%, 94%, 95%, 96%, 97%, 98%, 99%, or 100% sequence identity to an amino acid sequence chosen from: SEQ ID NO: 9 and SEQ ID NO: 10; or
(vii) a light chain (LC) comprising the amino acid sequence having at least 85%, 90%, 91%, 92%, 93%, 94%, 95%, 96%, 97%, 98%, 99%, or 100% sequence identity to the amino acid sequence of SEQ ID NO: 11; or
(viii) a HC comprising an amino acid sequence having at least 85%, 90%, 91%, 92%, 93%, 94%, 95%, 96%, 97%, 98%, 99%, or 100% sequence identity to an amino acid sequence chosen from: SEQ ID NO: 9 and SEQ ID NO: 10 and a LC comprising an amino acid sequence having at least 85%, 90%, 91%, 92%, 93%, 94%, 95%, 96%, 97%, 98%, 99%, or 100% sequence identity to the amino acid sequence of SEQ ID NO: 11; or
(ix) a HC comprising an amino acid sequence chosen from: SEQ ID NO: 9 and SEQ ID NO: 10; or
(x) a LC comprising the amino acid sequence of SEQ ID NO: 11; or
(xi) a HC comprising an amino acid sequence chosen from: SEQ ID NO: 9 and SEQ ID NO: 10 and a LC comprising the amino acid sequence of SEQ ID NO: 11.

Embodiment 2. The liquid pharmaceutical formulation as recited in Embodiment 1, wherein the anti-IL-1β antibody is present at a concentration of 135 mg/ml to 165 mg/ml.

Embodiment 3. The liquid pharmaceutical formulation as recited in Embodiments or 2, wherein the at least one buffering agent is present at a concentration of 10 mM to 30 mM, the at least one colloidal stabilizer is present at a concentration of 30 mM to 70 mM, the at least one antioxidant is present at a concentration of 1 to 20 mM, the at least one saccharide is present at a concentration of 70 to 150 mM, and the at least one surfactant is present at a concentration of 0.01% to 0.04% (w/v).

Embodiment 4. The liquid pharmaceutical formulation as recited in any one of Embodiments 1-3 comprising 150 mg/mL of the anti-IL-1β antibody, 20 mM of the at least one buffering agent, 50 mM of the at least one colloidal stabilizer, 10 mM of the at least one antioxidant, 110 mM of the at least one saccharide, and 0.02% to 0.04% (w/v) of the at least one surfactant.

Embodiment 5. The liquid pharmaceutical formulation as recited in any one of Embodiments 1-4, wherein the at least one buffering agent is chosen from: histidine; histidine HCl; and sodium phosphate.

Embodiment 6. The liquid pharmaceutical formulation as claimed in any one of Embodiments 1-5, wherein the at least one colloidal stabilizer is a free amino acid chosen from: arginine; arginine HCl; lysine; lysine HCl; and sodium chloride.

Embodiment 7. The liquid pharmaceutical formulation as claimed in any one of Embodiments 1-6, wherein the at least one saccharide acts as both a conformational stabilizer and a tonicity agent and is chosen from: sucrose, glucose; fructose; galactose; mannose; arabinose; xylose; erythrose; lactose; maltose; and trehalose.

Embodiment 8. The liquid pharmaceutical formulation as claimed in Embodiment 7, wherein the at least one saccharide is 110 mM sucrose, wherein the sucrose raises the tonicity of the formulation to an osmolality of 243-343 mOsmo/Kg.

Embodiment 9. The liquid pharmaceutical formulation as claimed in any one of Embodiments 1-8, wherein the at least one antioxidant is methionine.

Embodiment 10. The liquid pharmaceutical formulation as claimed in any one of Embodiments 1-9, wherein the at least one surfactant is a polysorbate.

Embodiment 11. The liquid pharmaceutical formulation as claimed in any one of Embodiments 1-10, wherein the polysorbate is PS80.

Embodiment 12. The liquid pharmaceutical formulation as claimed in any one of Embodiments 1-11, wherein the pH of the formulation is 5.7 to 7.

Embodiment 13. The liquid pharmaceutical formulation as claimed in any one of Embodiments 1-12, wherein the at least one buffering agent is histidine, the at least one colloidal stabilizer is arginine, the at least one saccharide is sucrose, the at least one antioxidant is methionine, and the at least one surfactant is PS80, and wherein the pH of the formulation is 5.7 to 6.5.

Embodiment 14. The liquid pharmaceutical formulation as claimed in any one of Embodiments 1-13, wherein the formulation is suitable for a route of administration chosen from: intravenous infusion and subcutaneous injection.

Embodiment 15. A liquid pharmaceutical formulation for delivery via subcutaneous injection comprising: 150 mg/mL of anti-IL-1β antibody; 20 mM histidine (11.6 mM histidine+8.4 mM histidine HCl); 50 mM arginine; 110 mM sucrose; 10 mM methionine; and 0.04% (w/v) polysorbate-80, wherein the anti-IL-1β antibody comprises: a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7 and a VL comprising an acid sequence chosen from: SEQ ID NO: 8 and SEQ ID NO: 12; a HC comprising an amino acid sequence chosen from: SEQ ID NO: 9 and SEQ ID NO: 10 SEQ ID NO: 9, and a LC comprising the amino acid sequence of SEQ ID NO: 11, wherein the pH of the formulation is 6.2.

Embodiment 16. A liquid pharmaceutical formulation for delivery via subcutaneous injection comprising: 150 mg/mL of anti-IL-1β antibody; 20 mM histidine (11.6 mM histidine+8.4 mM histidine HCl); 50 mM arginine; 110 mM sucrose; 10 mM methionine; and 0.02% to 0.04% (w/v) polysorbate-80, wherein the anti-IL-1β antibody comprises: a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7 and a VL comprising an acid sequence chosen from: SEQ ID NO: 8 and SEQ ID NO: 12; a HC comprising an amino acid sequence chosen from: SEQ ID NO: 9 and SEQ ID NO: 10 SEQ ID NO: 9, and a LC comprising the amino acid sequence of SEQ ID NO: 11, wherein the pH of the formulation is 6.0 to 6.4.

Embodiment 17. The liquid pharmaceutical formulation as claimed in Embodiment 16, wherein the formulation is delivered via intravenous infusion.

Embodiment 18. The liquid pharmaceutical formulation as claimed in any one of Embodiments 1-17, wherein the anti-IL-1β antibody comprises ≥95% monomeric antibody and ≤5% aggregated antibody.

Embodiment 19. The liquid pharmaceutical formulation as claimed in any one of Embodiments 1-18, wherein the anti-IL-1β antibody comprises 0.1% to 0.2% fragments.

Embodiment 20. The liquid pharmaceutical formulation as recited in any one of Embodiments 1-19, wherein the anti-IL-1β antibody comprises 40.0% to 65.0% of the main isoform as determined by imaged capillary isoelectric focusing (icIEF).

Embodiment 21. The liquid pharmaceutical formulation as claimed in any one of Embodiments 1-20, wherein the formulation may be stored at a temperature of 2° C. to 8° C. for 48 months without substantially impacting the purity of the formulation.

Embodiment 22. The liquid pharmaceutical formulation as recited in any one of Embodiments 1-20, wherein the anti-IL-1β antibody comprises ≥95% monomeric antibody and ≤5% aggregated antibody after storage at 2° C. to 8° C. for 48 months.

Embodiment 23. The liquid pharmaceutical formulation as recited in any one of Embodiments 1-20, wherein the anti-IL-1β antibody comprises ≤0.1% fragments after storage at 2° C. to 8° C. for 36 months.

Embodiment 24. The liquid pharmaceutical formulation as recited in any one of Embodiments 1-20, wherein the anti-IL-1β antibody comprises ≥95% monomeric antibody and ≤5% is aggregated antibody after storage at −85° C. to −65° C. for 36 months.

Embodiment 25. A liquid pharmaceutical formulation comprising:
a) a means for inhibiting IL-1β; and
b) at least one buffering agent
c) at least one colloidal stabilizer;
d) at least one antioxidant;
e) at least one saccharide; and
f) at least one surfactant.

Embodiment 26. The liquid pharmaceutical formulation as recited in Embodiment 25, wherein the means for inhibiting IL-1β is an anti-IL-1β antibody.

### BRIEF DESCRIPTION OF THE FIGURES

FIG. 1 is a viscosity curve from a shear-rate ramp (1 s⁻¹ to 10⁴ s⁻¹) for a solution of 198 mg/mL Antibody A in 10 mM sodium citrate and 150 mM NaCl at pH 6.7, as discussed in Example 1.

FIG. 2 shows the viscosity at 5° C. and 25° C. at a shear rate of 4000 s⁻¹ for different concentrations of Antibody A in 10 mM sodium citrate and 150 mM NaCl at pH 6.7, as discussed in Example 1.

FIG. 3 provides the results of peptide mapping by percent oxidation at each oxidation site in the heavy chain and light chain identified after H₂O₂ and AAPH incubation, where “n.d.” means not detected, as described in Example 3.

FIG. 4 shows the protein content by UV/Vis spectroscopy for formulations F1-F4 at T0 to 12 weeks at 5±3° C., 25±2° C. and 60%±5% relative humidity (RH), or 40° C.±2° C. and 75%±5% relative humidity, as discussed in Example 5.

FIG. 5 shows the clarity and opalescence of solutions (turbidity) for formulations F1-F4 at T0 to 12 weeks at 5±3° C., 25±2° C. and 60%±5% relative humidity (RH), or 40° C.±2° C. and 75%=5% relative humidity, as discussed in Example 5.

FIGS. 6A-6D shows the count of subvisible particles for formulations F1-F4 at T0 to 12 weeks at 5±3° C., 25±2° C. and 60%±5% relative humidity (RH), or 40° C.±2° C. and 75%±5% relative humidity for particles ≥2 μm (FIG. 6A), particles ≥5 μm (FIG. 6B), particles ≥10 μm (FIG. 6C), and particles ≥25 μm (FIG. 6D), as discussed in Example 5.

FIGS. 7A-7D provides the results of size exclusion HPLC (SE-HPLC) for formulations F1-F4 at T0 to 12 weeks at 5±3° C., 25±2° C. and 60%±5% relative humidity (RH), or 40° C.±2° C. and 75%±5% relative humidity by the % area of the main peak (FIG. 7A), the % area of the high molecular weight species (HMWS) (FIG. 7B), and the % area of the low molecular weight species (LMWS) (FIG. 7C). FIG. 7D provides an overlay of SE-HPLC chromatograms for formulations F1-F4 after 12 weeks at 40° C.±2° C. and 75%±5% relative humidity, as discussed in Example 5.

FIGS. 8A-8D provides the results of imaged capillary isoelectric focusing (icIEF) for formulations F1-F4 at T0 to 12 weeks at 5±3° C., 25±2° C. and 60%±5% relative humidity (RH), or 40° C.±2° C. and 75%±5% relative humidity by the % area of the main peak (FIG. 8A), the % area of the basic variants (FIG. 8B), and the % area of the acidic variants (FIG. 8C). FIG. 8D provides an overlay of the icIEF data for formulations F1-F4 after 12 weeks at 40° C.±2° C. and 75%±5% relative humidity, as discussed in Example 5.

FIGS. 9A-9D shows the results of reverse phase HPLC (RP-HPLC) for formulations F1-F4 at T0 to 12 weeks at 5±3° C., 25±2° C. and 60%±5% relative humidity (RH), or 40° C.±2° C. and 75%±5% relative humidity by the % area of the main peak under non-reducing conditions (FIG. 9A), by the % area of the LC of Antibody A under reducing conditions (FIG. 9B), by the % area of the HC of Antibody A under reducing conditions (FIG. 9C), and by the % area of the LC plus the % area of the HC of Antibody A under reducing conditions (FIG. 9D), as discussed in Example 5.

FIGS. 10A-10E shows the results of capillary electrophoresis sodium dodecyl sulfate (CE-SDS) by Labchip for formulations F1-F4 at T0 to 12 weeks at 5±3° C., 25±2° C. and 60%±5% relative humidity (RH), or 40° C.±2° C. and 75%±5% relative humidity by % area of the main peak under non-reducing conditions (FIG. 10A), by the % area of the LC of Antibody A under reducing conditions (FIG. 10B), by the % area of the HC of Antibody A under reducing conditions (FIG. 10C). FIG. 10D provides overlays of CE-SDS data under reducing conditions for formulations F1-F4 after 12 weeks at 40° C.±2° C. and 75%±5% relative humidity. FIG. 10E provides overlays of CE-SDS data under non-reducing conditions for formulations F1-F4 after 12 weeks at 40° C.±2° C. and 75%±5% relative humidity, as discussed in Example 5.

FIG. 11 shows the polysorbate 80 (PS80) content in % (w/v) determined by a fluorescent micelle assay for formulations F1-F4 at T0 to 12 weeks at 5±3° C., 25±2° C. and 60%±5% relative humidity (RH), or 40° C.±2° C. and 75%±5% relative humidity, as discussed in Example 5.

FIG. 12 shows the pH for formulations F1-F4 at T0 to 12 weeks at 5±3° C., 25±2° C. and 60%±5% relative humidity (RH), or 40° C.±2° C. and 75%±5% relative humidity, as discussed in Example 5.

FIG. 13 shows the total protein content (mg/mL) determined by UV/vis for formulations F3 and F4 at T0 at room temperature (25° C.); at T6M, T9M, T12M, T18M, and T24M at 5±3° C.; and at T6M at 25±2° C. and 60%±5% relative humidity (RH).

FIG. 14 shows the clarity and opalescence of solution (turbidity in NTU) for formulations F3 and F4 at T0 at room temperature (25° C.); at T6M, T9M, T12M, T18M, and T24M at 5±3° C.; and at T6M at 25±2° C. and 60%±5% relative humidity (RH).

FIGS. 15A-15D shows the subvisible particle count for formulations F3 and F4 at T0 at room temperature (25° C.); at T6M, T9M, T12M, T18M, and T24M at 5±3° C.; and at T6M at 25±2° C. and 60%±5% relative humidity (RH) for particles ≥2 μm (#/mL) (FIG. 15A), particles ≥5 μm (#/mL) (FIG. 15B), particles ≥10 μm (#/mL) (FIG. 15C), and particles ≥25 μm (#/mL) (FIG. 15D), as discussed in Example 5.

FIGS. 16A-16C provides the purity results by size exclusion HPLC (SE-HPLC) for formulations F3 and F4 at T0 at room temperature (25° C.); at T6M, T9M, T12M, T18M, and T24M at 5±3° C.; and at T6M at 25±2° C. and 60%±5% relative humidity (RH) by the % area of the main peak (FIG. 16A) and the % area of the high molecular weight species (HMWS) (FIG. 16B). The low molecular weight species (LMWS) for F3 and F4 were either not detected or under the limit of detection at the times and under the conditions tested. FIG. 16C provides an overlay of SE-HPLC data for formulations F3 and F4 after 12 months at 5±3° C., as discussed in Example 5.

FIGS. 17A-17D provides the results of imaged capillary isoelectric focusing (icIEF) for formulations F3 and F4 at T0 at room temperature (25° C.); at T6M, T9M, T12M, T18M, and T24M at 5±3° C.; and at T6M at 25±2° C. and 60%±5% relative humidity (RH) by the % area of the main peak (FIG. 17A), the % area of the acidic variants (FIG. 17B), and the % area of the basic variants (FIG. 17C). FIG. 17D provides an overlay of icIEF data for formulations F3 and F4 after 12 months at 5±3° C., as discussed in Example 5.

FIGS. 18A-18F shows the results of reverse phase HPLC (RP-HPLC) for formulations F3 and F4 at T0 at room temperature (25° C.); at T6M, T9M, T12M, T18M, and T24M at 5±3° C.; and at T6M at 25±2° C. and 60%±5% relative humidity (RH) by the % area of the main peak under non-reducing conditions (FIG. 18A), by the % area of the LC of Antibody A under reducing conditions (FIG. 18B), by the % area of the HC of Antibody A under reducing conditions (FIG. 18C), and by the % area of the LC plus the % area of the HC of Antibody A under reducing conditions (FIG. 18D). FIG. 18E provides an overlay of RP-HPLC data under non-reducing conditions for formulations F3 and F4 after 12 months at 5±3° C. FIG. 18F provides an overlay of RP-HPLC data under reducing conditions for formulations F3 and F4 after 12 months at 5±3° C., as discussed in Example 5.

FIGS. 19A-19E shows the results of capillary electrophoresis sodium dodecyl sulfate (CE-SDS) by Labchip for formulations F3 and F4 at T0 at room temperature (25° C.); at T6M, T9M, T12M, T18M, and T24M at 5±3° C.; and at T6M at 25±2° C. and 60%±5% relative humidity (RH) by % area of the main peak under non-reducing conditions (FIG. 19A), by the % area of the LC of Antibody A under reducing conditions (FIG. 19B), by the % area of the HC of Antibody A under reducing conditions (FIG. 19C). FIG. 19D provides an overlay of CE-SDS data under non-reducing conditions for formulations F3 and F4 after 12 months at 5±3° C. FIG. 19E provides an overlay of CE-SDS data under reducing conditions for formulations F3 and F4 after 12 months at 5±3° C., as discussed in Example 5.

FIG. 20 shows the polysorbate 80 (PS80) content in % (w/v) determined by a fluorescent micelle assay for formulations F3 and F4 at T0 at room temperature (25° C.); at T6M, T9M, T12M, T18M, and T24M at 5±3° C.; and at T6M at 25±2° C. and 60%±5% relative humidity (RH), as discussed in Example 5.

FIG. 21 shows the pH for formulations F3 and F4 at T0 at room temperature (25° C.); at T6M, T9M, T12M, T18M, and T24M at 5±3° C.; and at T6M at 25±2° C. and 60%±5% relative humidity (RH), as discussed in Example 5.

FIG. 22 provides the results of stability testing of Formulation F3 of Antibody A at 5° C.±3° C. in inverted vials from T0 to 36 months as described in Example 6.

FIG. 23 provides the results of stability testing of Formulation F3 of Antibody A at 5° C.±3° C. in upright vials from T0 to 48 months as described in Example 6.

FIG. 24 provides the results of stability testing of Formulation F3 of Antibody A at 25° C./60% relative humidity from T0 to 12 months as described in Example 6.

FIG. 25 provides the results of stability testing of Formulation F3 of Antibody A at 40° C./75% relative humidity from T0 to 6 months as described in Example 6.

FIG. 26 provides the results of stability testing of Formulation F3 of Antibody A at −75° C.±10° C. (−65° C. or below) from T0 to 36 months as described in Example 7.

FIG. 27 provides the results of stability testing of Formulation F3 of Antibody A at 5° C.±3° C. (±5° C.) from T0 to 12 months as described in Example 7.

FIG. 28 provides the results of accelerated stability testing of Formulation F3 of Antibody A at 25° C.±2° C./60%±5% relative humidity (±25° C./60% RH) from T0 to 6 months as described in Example 7.

### DETAILED DESCRIPTION OF THE INVENTION

The following definitions are provided to facilitate an understanding of the invention. They are not intended to limit the invention in any way.

### Definitions

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain (HCD1, HCDR2, and HCDR3, respectively), and at least CDR1, CDR2, and CDR3 of a light chain (LCDR1, LCDR2, and LCDR3, respectively), wherein the antibody is capable of binding to its antigen. The term “antibody” includes, but is not limited to, fragments that are capable of binding to antigen, such as Fv, single-chain Fv (scFv), Fab, Fab′, and (Fab′) 2. The term “antibody” also includes, but is not limited to, chimeric antibodies, humanized antibodies, human antibodies, and antibodies of various species such as mouse, cynomolgus monkey, etc.

The term “heavy chain” or “HC” are synonymous, and both refer to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” and “VH” are synonymous, and both refers to a region comprising a HCDR 1, framework region (FR) 2, HCDR2, FR3, and HCDR3 of the HC. In some embodiments, a VH also comprises at least a portion of an FR1 and/or at least a portion of an FR4. In some embodiments, a HCDR1 corresponds to Kabat residues 31 to 35; a HCDR2 corresponds to Kabat residues 50 to 65; and a HCDR3 corresponds to Kabat residues 95 to 102. See, e.g., Kabat Sequences of Proteins of Immunological Interest (1987 and 1991, NIH, Bethesda, Md.).

The terms “light chain” or “LC” are synonymous, and both refer to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “light chain variable region” or “VL” are synonymous, and both refer to a region comprising a LCDR1, FR2, LCDR2, FR3, and LCDR3. In some embodiments, a VL also comprises an FR1 and/or an FR4. In some embodiments, a LCDR1 corresponds to Kabat residues 24 to 34; a LCDR2 corresponds to Kabat residues 50 to 56; and a LCDR3 corresponds to Kabat residues 89 to 97. See, e.g., Kabat Sequences of Proteins of Immunological Interest (1987 and 1991, NIH, Bethesda, Md.).

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a Fab, an scFv, a (Fab′)₂, etc.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “buffering agent” refers to an agent that helps stabilize pH. For example, the buffering agent may be a weak acid or base that maintains the pH of a solution by absorbing excess protons or hydroxide ions, thereby preventing large, rapid changes in acidity when other acids or bases are added to the solution.

The term “free amino acid” refers to amino acids not bound to other amino acids to form peptide bonds.

The term “colloidal stabilizer” refers to an agent that decreases intermolecular attractive forces and/or increases intermolecular repulsive forces between other molecules. For example, the colloidal stabilizer may be a substance that prevents dispersed particles in a colloid from aggregating or separating by creating a repulsive force between them, either through electrostatic repulsion, i.e., charges or through steric hindrance, which creates a physical barrier between the particles in the colloid.

The term “saccharide” refers to a compound that has a general formula (CH₂O)ₙ and derivatives thereof, including, for example, monosaccharides, disaccharides, trisaccharides, polysaccharides, sugar alcohols, reducing sugars, and nonreducing sugars.

The term “antioxidant” refers to an agent that inhibits or prevents the oxidation of other molecules.

The term “surfactant” refers to an agent that lowers the surface tension of a liquid.

The term “histidine” refers to L-histidine, and the term “histidine HCl” refers to L-histidine monohydrochloride.

The term “Arginine” refers to L-arginine, and “arginine HCl” refers to L-arginine monohydrochloride.

The terms “polysorbate-80” and “PS80” are used synonymously, and both refer to polyoxyethylene (80) sorbitan monooleate.

The term “isotonic” refers to a solution having the same osmotic pressure as a reference solution.

Where a range of values is provided, each numerical value between the upper and lower limits of the range is contemplated and disclosed herein and the endpoints of the range are also contemplated as included in the range.

### A. IL-1β Antibodies

In some embodiments, anti-IL-1β antibodies suitable for use in the liquid pharmaceutical formulations include IgG4-type anti-IL-1β antibodies.

In some embodiments, anti-ILI-1B antibodies suitable for use in the formulations are humanized.

In some embodiments, anti-IL-1β antibodies suitable for use in the formulations disclosed herein include anti-IL-1β antibodies comprising: a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6.

In other embodiments, anti-IL-1β antibodies suitable for use in the formulations disclosed herein include anti-IL-1β antibodies comprising a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7 and a VL comprising an acid sequence chosen from: SEQ ID NO: 8 and SEQ ID NO: 12; a HC comprising an amino acid sequence chosen from: SEQ ID NO: 9 and SEQ ID NO: 10 SEQ ID NO: 9, the VH without a leader sequence; and a LC comprising an amino acid sequence of SEQ ID NO: 11.

In some embodiments, anti-IL-1 antibodies suitable for use in the formulations include anti-IL-1β antibodies comprising a VH comprising the acid sequence of SEQ ID NO: 7.

In some embodiments, anti-IL-1β antibodies suitable for use in the formulations disclosed herein include anti-IL-1β antibodies comprising a VL comprising an amino acid sequence chosen from: SEQ ID NO: 8 and SEQ ID NO: 12.

In some embodiments, anti-IL-1β antibodies suitable for the formulations include anti-IL-1β antibodies comprising: a VH comprising the acid sequence of SEQ ID NO: 7; and a VL comprising an acid sequence chosen from: SEQ ID NO: 8 and SEQ ID NO: 12.

In some embodiments, anti-IL-1β antibodies suitable for use in the formulations include anti-IL-1β antibodies comprising: a HC comprising an amino acid sequence having at least 85%, 90%, 91%, 92%, 93%, 94%, 95%, 96%, 97%, 98%, 99%, or 100% sequence identity to an amino acid sequence chosen from: SEQ ID NO: 9 and SEQ ID NO: 10.

In some embodiments, the anti-IL-1β antibodies suitable use in the formulations include anti-IL-1β antibodies comprising a LC comprising an amino acid sequence having at least 85%, 90%, 91%, 92%, 93%, 94%, 95%, 96%, 97%, 98%, 99%, or 100% sequence identity to the amino acid sequence of SEQ ID NO: 11.

In some embodiments, anti-IL-1β antibodies suitable for use in the formulations include anti-IL-1β antibodies comprising: a HC comprising an amino acid sequence having at least 85%, 90%, 91%, 92%, 93%, 94%, 95%, 96%, 97%, 98%, 99%, or 100% sequence identity to an amino acid sequence chosen from SEQ ID NO: 9 and SEQ ID NO: 10; and a LC comprising an amino acid sequence comprising at least 85%, 90%, 91%, 92%, 93%, 94%, 95%, 96%, 97%, 98%, 99%, or 100% sequence identity to the amino acid sequence of SEQ ID NO: 11.

In some embodiments, anti-IL-1β antibodies suitable for use in the formulations include anti-IL-1β antibodies comprising a HC comprising an acid sequence chosen from: SEQ ID NO: 9 and SEQ ID NO: 10.

In some embodiments, anti-IL-1β antibodies suitable for use in the formulations include anti-IL-1β antibodies comprising a LC comprising an amino acid sequence chosen from: SEQ ID NO: 11.

In some embodiments, anti-IL-1β antibodies suitable for use in the formulations include anti-IL-1β antibodies comprising: a HC comprising an acid sequence chosen from: SEQ ID NO: 9 and SEQ ID NO: 10; and a LC comprising the acid sequence of SEQ ID NO: 11.

### B. Exemplary Formulations for Anti-IL-1β Antibodies

In some embodiments, the liquid pharmaceutical formulation comprises: an anti-IL-1β antibody as described herein in Section A, at least one buffering agent; at least one colloidal stabilizer; at least one antioxidant; at least one saccharide; and at least one surfactant. In some embodiments, the anti-IL-1β antibody is the antibody described and referred to herein as “Antibody A.”

In some embodiments, the formulation comprises a concentration of Antibody A of 100 mg/mL to 200 mg/mL. In some embodiments the formulation comprises a concentration of Antibody A of 135 mg/ml to 165 mg/ml. In some embodiments, the formulation comprises 150 mg/mL of Antibody A.

In some embodiments, the at least one buffering agent suitable for use in the formulation is chosen from: histidine; histidine HCl; and sodium phosphate.

In some embodiments, the at least one buffering agent sodium phosphate. In some embodiments, the at least one buffering agent is present at a concentration of 10 mM to 30 mM. In some embodiments, the at least one buffering agent is present at a concentration of 20 mM.

In some embodiments, the at least one colloidal stabilizer suitable for use in the formulation is chosen from: sodium chloride; free amino acids; In other embodiments, the at least one colloidal stabilizer is chosen from: arginine; arginine HCl; lysine; lysine HCl; and sodium chloride. In some embodiments, the colloidal stabilizer is present at a concentration of 30 mM to 70 mM or 40 mM to 60 mM. In other embodiments, the colloidal stabilizer is present at a concentration of 50 mM.

In some embodiments, the at least one saccharide suitable for use in the formulation is chosen from: sucrose; glucose; fructose; galactose; mannose; arabinose; xylose; erythrose; lactose; maltose; and trehalose, and wherein the at least one saccharide acts as both a conformational stabilizer and as a tonicity agent, wherein the at least one saccharide raises the tonicity of the formulation, resulting in an osmolality of the formulation to physiological conditions of 243-343 mOsmo/Kg.

In other embodiments, the formulation further comprises at least one tonicity agent chosen from: sucrose and trehalose.

In some embodiments, the at least one saccharide is present at a concentration of: 70 mM to 150 mM, 80 mM to 140 mM, 90 mM to 130 mM, 100 mM to 120 mM, or 105 mM to 115 mM. In another embodiment, the saccharide is sucrose and is present at a concentration of 110 mM.

In some embodiments, the at least one antioxidant suitable for use in the formulation is methionine. In some embodiments, the antioxidant is present at a concentration of 1 mM to 20 mM, 5 mM to 15 mM, or 8 mM to 12 mM. In yet another embodiment, the at least one antioxidant is present at a concentration of 10 mM.

In some embodiments, the at least one surfactant suitable for use in the formulation is chosen from: a polysorbate. In some embodiments, the at least one surfactant is PS80. In some embodiments, the at least one surfactant is present at a concentration of 0.01% (w/v) to 0.1% (w/v), or 0.02% (w/v) to 0.06% (w/v). In other embodiments, the surfactant is present at a concentration of 0.04% (w/v).

In some embodiments, the pH of the formulation is 5.7 to 6; 6 to 7; 6 to 6.1; 6.1 to 6.2; 6.2 to 6.3; 6.3 to 6.4; 6.4 to 6.5; 6.5 to 6.6; 6.6 to 6.7; 6.7 to 6.8; 6/8 to 6/9; and 6.9 to 7. In other embodiments, the pH of the formulation is 6.2.

In other embodiments, the at least one buffering agent is histidine, the at least one colloidal stabilizer is arginine, the at least one saccharide is sucrose, the at least one antioxidant is methionine, and the at least one surfactant is PS80, wherein the pH of the formulation is 5.7 to 6.5.

In some embodiments, the formulation comprises 100 mg/mL to 200 mg/mL of Antibody A, 10 mM to 30 mM of at least one buffering agent chosen from: histidine/histidine HCl; and sodium phosphate; 40 mM to 60 mM of at least one colloidal stabilizer chosen from: sodium chloride, arginine/arginine HCl, and lysine/lysine HCl; 100 mM to 120 of at least one saccharide chosen from: sucrose, glucose, fructose, galactose, mannose, arabinose, xylose, erythrose, lactose, maltose, and trehalose; 5 mM to 15 mM antioxidant such as methionine, and 0.02% (w/v) to 0.6% (w/v) of at least one surfactant such as polysorbate-80, wherein the pH of the formulation is 6 to 7.

In some embodiments, the formulation is suitable for a route of administration chosen from: intravenous infusion and subcutaneous injection. In other embodiments, the formulation is administered via subcutaneous injection. Any of the liquid pharmaceutical formulations disclosed herein may be delivered via subcutaneous injection or via intravenous infusion with the same excipients and in the same amounts as disclosed herein.

In some embodiments, the liquid pharmaceutical formulation is delivered via subcutaneous injection and comprises: 150 mg/ml of anti-IL-1β antibody; 20 mM histidine (11.6 mM histidine+8.4 mM histidine HCl), 50 mM arginine; 110 mM sucrose; 10 mM methionine; and 0.04% (w/v) polysorbate-80, wherein the anti-IL-1β antibody comprises: a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7 and a VL comprising an acid sequence chosen from: SEQ ID NO: 8 and SEQ ID NO: 12; a HC comprising an amino acid sequence chosen from: SEQ ID NO: 9 and SEQ ID NO: 10 SEQ ID NO: 9, and a LC comprising the amino acid sequence of SEQ ID NO: 11, wherein the pH of the formulation is 6.2. In some embodiments, formulation comprises 150 mg/mL of Antibody A, about 20 mM histidine/histidine HCl, about 50 mM arginine/arginine HCl, about 110 mM sucrose, about 10 mM methionine, and about 0.02% to 0.04% (w/v) PS80, wherein the pH of the formulation is about 6, or about 6.2, or about 6.4. In some embodiments, the formulation comprises an anti-IL-1β antibody that comprises ≥95% monomeric antibody and ≤5% aggregated antibody.

In some embodiments, the formulation is suitable for a route of administration chosen from: intravenous infusion and/or subcutaneous injection.

In some embodiments, the formulation may be stored at a temperature ranging from 2° C. to 8° C. for six months, nine months, twelve months, eighteen months, twenty-four months, thirty-six months, or forty-eight months without substantially impacting the purity of the formulation. In another embodiment, the formulation may be stored at 2° C. to 8° C. for 48 months without substantially impacting the purity of the formulation.

In some embodiments, the formulation may be stored at ≤−65° C. for six months, nine months, twelve months, eighteen months, twenty-four months, or thirty-six months without substantially impacting the purity of the formulation. In some embodiments, at temperatures at or below (≤)−65° C., Antibody A can be stored >36M while maintaining ≥95% monomeric and ≤5% aggregated antibody.

In some embodiments, the formulation may be stored at ≤−65° C. for up to thirty-six months and then thawed and maintained at about 5° C. for six months, nine months, twelve months, eighteen months, or twenty-four months without substantially impacting the purity of the formulation.

In some embodiments, the formulation comprises an Antibody A comprises ≥95%, ≥97%, ≥98%, or ≥99% monomeric species as determined by size-exclusion high performance liquid chromatography (SE-HPLC, also called gel permeation HPLC or GP-HPLC). In some embodiments, Antibody A comprises ≥95%, monomeric species as determined by size-exclusion high performance liquid chromatography.

In some embodiments, the formulation comprises Antibody A comprising ≤5% aggregated antibody (e.g., dimers, trimers, and larger aggregates of Antibody A), ≤3% aggregate antibody, ≤2% aggregated antibody, or ≤1% aggregated antibody. In some embodiments, Antibody A comprises ≤5% aggregated antibody.

In some embodiments, the anti-IL-1β antibodies comprise ≥95% monomeric species and ≤5% aggregated antibody as determined by size-exclusion high performance liquid chromatography.

In other embodiments, Antibody A can be stored at 2° C. to 8° C. for up to 48 months while maintaining ≥95% monomeric antibody and ≤5% aggregated antibody.

In some embodiments, Antibody A comprises 0.1% to 0.2% fragments. In some embodiments, Antibody A comprises 0.1% fragments.

In some embodiments, 40.0%-60.0% of the anti-IL-1β antibodies in the formulation comprise the main isoform as observed by imaged capillary isoelectric focusing (icIEF). In some embodiments, 40.0%-60.0% of the anti-IL-1β antibodies in the formulation are in the main isoform as observed by icIEF.

In some embodiments the formulation comprises:
a. a means for inhibiting IL-1β;
b. at least one buffering agent;
c. at least one colloidal stabilizer;
d. at least one antioxidant;
e. at least one saccharide; and
f. at least one surfactant.

In other embodiments, the means for inhibiting IL-1β is an anti-IL-1β antibody.

### EXAMPLES

The following examples are provided to illustrate certain disclosed embodiments and are not to be construed as limiting the scope of this disclosure in any way.

Antibody A was manufactured and provided for testing as a solution containing a nominal protein concentration of 61.2 mg/mL and 10 mM sodium citrate/150 mM NaCl at pH 6.7, referred to herein as the Stock Solution of Antibody A. The molecular weight of Antibody A was identified as 145.6 kDa. The extinction coefficient of Antibody A was 1.46 mL/(mg*cm). Antibody A was stored at 2° C. to 8° C. prior to testing.

Antibody A is an IgG4-type anti-IL-1β antibody comprising a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7; a VL comprising an acid sequence chosen from: SEQ ID NO: 8 and SEQ ID NO: 12 (an alternative VL); a HC comprising an amino acid sequence chosen from: SEQ ID NO: 9 and SEQ ID NO: 10 (the VH without a leader sequence); and a LC comprising an amino acid sequence of SEQ ID NO: 11.

### Example 1: Viscosity Versus Concentration Profiling

A viscosity-concentration profile was determined for Antibody A at five concentrations and at temperatures of 5° C. and 25° C. to determine the upper concentration limit for development of a liquid formulation comprising Antibody A.

The concentration of Antibody A was increased to 200 mg/mL by spin filtration. Protein concentration was determined to be 198 mg/mL using SoloVPER (C Technologies). The 198 mg/mL solution was used to prepare a dilution series containing approximately 180 mg/mL, 120 mg/mL, 80 mg/mL, and 40 mg/mL protein, and the protein concentration was checked again by SoloVPER and determined to be 166 mg/mL, 126 mg/mL, 84 mg/mL, and 41 mg/mL.

The rheologic behavior of the 198 mg/mL protein solution was assessed with a shear-rate ramp using a cone/plate-equipped rheometer (Rheometer MCR102, Anton Paar). From these data, the optimal shear rate (Newtonian flow-range) for the viscosity measurements was determined. The viscosities of the protein solutions from the dilution series were then measured at this determined shear rate (4000 s⁻¹) using the same rheometer at 5° C. and 25° C. The measurements at 25° C. were performed as duplicates, and the arithmetic mean values were calculated to acquire additional data at the injection/administration temperature, while the measurements at 5° C. were performed as single measurements.

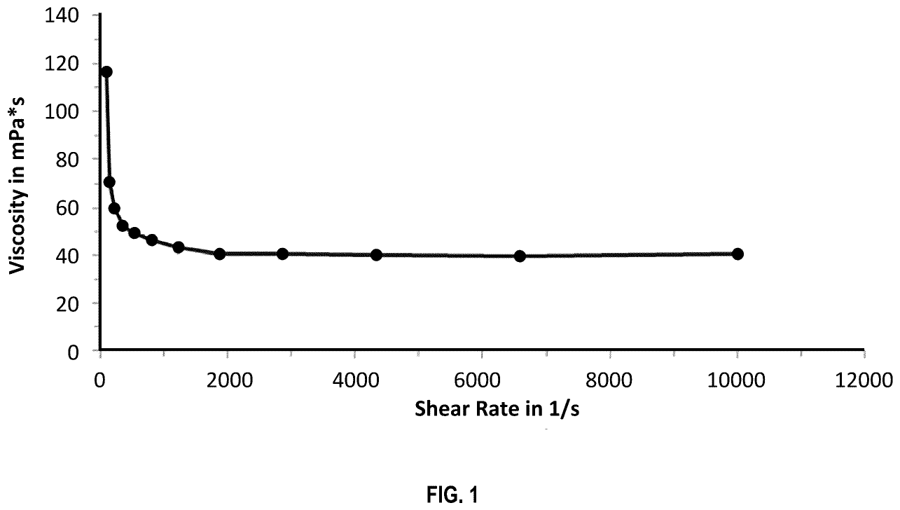

The 198 mg/mL solution of Antibody A in 10 mM sodium citrate and 150 mM NaCl at pH 6.7 exhibited shear-thinning flow behavior. The viscosity curve showed constant viscosity (Newtonian flow range) at shear rates above 2000 s⁻¹, as shown in FIG. 1. A shear rate of 4000 s⁻¹ was selected for viscosity measurements in all further experiments.

The viscosities of solutions of Antibody A at 166 mg/mL, 126 mg/mL, 84 mg/mL, and 41 mg/mL in 10 mM sodium citrate and 150 mM NaCl at pH 6.7 were determined at 5° C. and 25° C.

TABLE 1

| Protein concentration (mg/mL) | 41 | 84 | 126 | 166 | 198 |
|---|---|---|---|---|---|
| Viscosity (mPa*s) at 5° C. | 3 | 4 | 8 | 22 | 57 |
| Viscosity (mPa*s) at 25° C., replicate 1 | 1 | 2 | 4 | 10 | 24 |
| Viscosity (mPa*s) at 25° C., replicate 2 | 2 | 3 | 5 | 11 | 23 |
| Average Viscosity (mPa*s) at 25° C. | 1 | 2 | 5 | 10 | 23 |

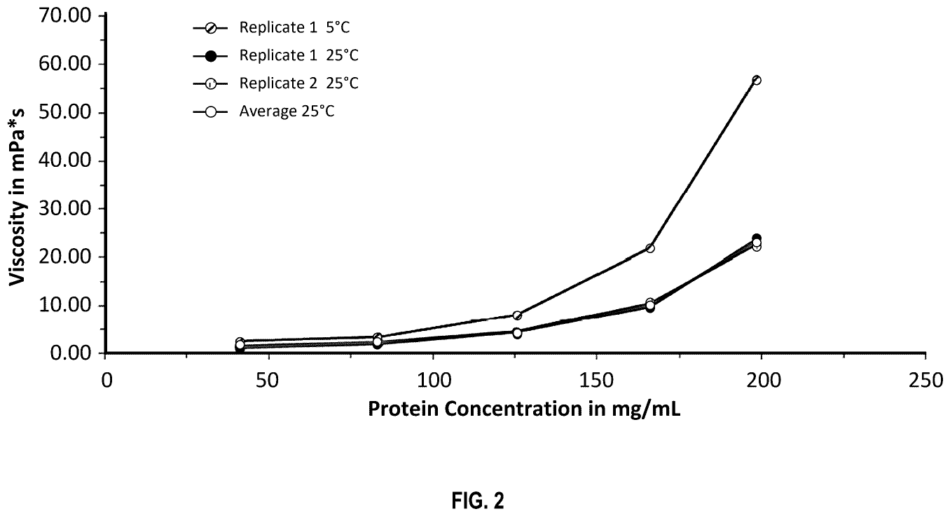

FIG. 2 provides a graph of viscosity versus protein concentration at 5° C. and 25° C. for the tested solutions. The measured steady-state viscosities showed a typical hyperbolic profile with an exponential increase in viscosity for protein concentrations above 150 mg/mL. Overall, the viscosity appeared to be in an acceptable range for a target concentration of 150 mg/mL.

### Example 2: Viscosity Screening of Different Formulations

A viscosity screening study of several Antibody A formulations was performed to assess the impact of buffering agents and excipients on formulations comprising Antibody A.

The formulations listed in Table 2 were prepared as follows. A solution of 60 mg/mL Antibody A in 10 mM sodium citrate/150 mM NaCl solution was buffer exchanged into the respective buffers by spin filtration. The concentration of Antibody A was increased by spin filtration before being diluted to the target concentration of 180 mg/mL, at which point the protein concentration and pH were checked.

TABLE 2

| | | | | | Protein |
|---|---|---|---|---|---|
| | | | | | Concentration |
| No. | Buffer | pH | Excipient 1 | Excipient 2 | (mg/mL) |
| A1 | 20 mM | 6.2 | — | — | 181 |
| | Histidine | | | | |
| | HCl | | | | |
| A2 | 20 mM | 6.5 | — | — | 181 |
| | Histidine | | | | |
| | HCl | | | | |
| A3 | 20 mM | 6.5 | — | — | 180 |
| | sodium | | | | |
| | citrate | | | | |
| A4 | 20 mM | 6.5 | — | — | 180 |
| | sodium | | | | |
| | succinate | | | | |
| A5 | 20 mM | 6.5 | 150 mM | — | 180 |
| | Histidine | | NaCl | | |
| | HCl | | | | |
| A6 | 20 mM | 6.5 | 50 mM | 150 mM | 179 |
| | Histidine | | Arginine | sucrose | |
| | HCl | | HCl | | |
| A7 | 20 mM | 6.5 | 100 mM | 75 mM | 183 |
| | Histidine | | Arginine | sucrose | |
| | HCl | | HCl | | |
| A8 | 20 mM | 6.2 | 50 mM | 150 mM | 180 |
| | Histidine | | Arginine | sucrose | |
| | HCl | | HCl | | |

The viscosity screening was performed using the same cone/plate-equipped rheometer as described in Example 1 at a shear rate of 4000 s⁻¹ and temperature of 25° C. Each formulation was tested twice, and the average viscosity was calculated. The results are tabulated in Table 3.

TABLE 3

| No. | A1 | A2 | A3 | A4 | A5 | A6 | A7 | A8 |
|---|---|---|---|---|---|---|---|---|
| Viscosity | 26 | 29 | 18 | 24 | 14 | 16 | 13 | 17 |
| replicate | | | | | | | | |
| 1 (mPa*s) | | | | | | | | |
| Viscosity | 27 | 30 | 19 | 25 | 14 | 16 | 12 | 16 |
| replicate | | | | | | | | |
| 2 (mPa*s) | | | | | | | | |
| Viscosity | 26 | 30 | 19 | 24 | 14 | 16 | 13 | 16 |
| averaged | | | | | | | | |
| (mPa*s) | | | | | | | | |

The viscosities of the screened formulations concentrated to about 180 mg/mL ranged from 13 mPa*s to 30 mPa*s (Table 3). Of the three tested buffering agents tested at pH 6.5 in formulations A2-A4, sodium citrate (Na-citrate) (formulation A3) exhibited lowest viscosity at a set pH, while the formulation with the histidine-HCl buffering agent (formulation A2) provided the highest viscosity. Reducing the pH from 6.5 to 6.2 provided a modest decrease in viscosity for formulation A1 versus A2.

Comparing the formulations comprising histidine-HCl at pH 6.5 (A2 and A5-A7), the data show that adding 150 mM NaCl (A5), 50 mM arginine HCl and 150 mM sucrose (A6), or 100 mM arginine-HCl and 75 mM sucrose (A7) reduced viscosity from about 30 mPa*s in A2 to about 14 mPa*s in A5, 16 mPa*s in A6, and 13 mPa*s in A7.

Comparing the formulations comprising histidine-HCl at pH 6.2 (A1 and A8), the data show that adding 50 mM arginine HCl and 150 mM sucrose reduced viscosity from 26 mPa*s (A1) to 16 mPa*s (A8).

Without wishing to be bound by any theory, it is expected that the decrease in viscosity in these formulations observed for formulations comprising NaCl or arginine HCl is due to the increased ionic strength of these solutions.

### Example 3: Susceptibility of Antibody A to Posttranslational Modifications

The susceptibility of Antibody A to deamidation, oxidation, clipping, and aggregation was evaluated.

Antibody A was subjected to a forced degradation stress panel, including forced deamidation at high pH, forced clipping (succinimide formation) at low pH, forced oxidation of methionine residues by hydrogen peroxide (H₂O₂), and forced oxidation of tryptophan residues by 2,2′-azobis(2-amidinopropane) dihydrochloride (AAPH).

An appropriate stock solution of Antibody A was prepared and pH or chemical stress agents were added. Samples were incubated at 40° C. for a distinct time, then frozen and kept at ≤−65° C. until analysis. To account for any effects due to thermal stress, control samples at standard pH and without chemical stress agents were incubated at 40° C. for the same duration as stressed samples. A stress control solution, as well as Antibody A control, were frozen immediately after preparation (T0). The antibody was evaluated after incubation under the various stress conditions for durations T_{A}, T_{B}, and/or T_{C}, as listed in Table 4.

TABLE 4

| | | Stress | Low Stress | High Stress |
|---|---|---|---|---|
| Condition | pH | Agent | Level | Level |
| Clipping | ~4 | N/A | T_{B} | T_{C} |
| Control | 6 | N/A | T_{B} | T_{C} |
| Deamidation | ~8 | N/A | T_{B} | T_{C} |
| Methionine | Same as | H₂O₂ | T_{A} | T_{A} |
| oxidation | control | | | |
| Tryptophan | Same as | AAPH | T_{A} | T_{A} |
| oxidation | control | | | |

Table 5 identifies the analytical techniques used to evaluate the purity of the various samples under the different conditions.

TABLE 5

| | | | Methionine | Tryptophan |
|---|---|---|---|---|
| Assay | Clipping | Deamidation | oxidation | oxidation |
| SE-HPLC | X | X | X | X |
| CE-SDS | X | | | |
| icIEF | | X | | |
| RP-HPLC | X | | X | X |
| LC-MS | | | X | X |

SE-HPLC (also called gel permeation HPLC or GP-HPLC) was performed to estimate the amounts of monomers, dimers, and aggregates. icIEF was performed using the iCE280 and iCE3. All samples were further subjected to peptide mapping by LC-MS.

### Results

Clipping. The results of the clipping study are reported in Table 6. Samples subjected to forced clipping (succinimide formation) at pH 4 showed considerable aggregation by SE-HPLC, generating about 40% aggregates compared to essentially no aggregates observed at pH 6. The low molecular weight species were below the limit of detection in all samples.

The CE-SDS results implied a decreasing trend for the relative amount of the HC for the pH 4 samples, decreasing from about 66% at T0 to about 64% after T_{C}. The sample subjected to pH 4 for T_{C} also showed a minor decrease in the amount of unmodified antibody by non-reducing RP-HPLC and the amount of HC observed by reducing RP-HPLC.

Table 6 summarizes the results of testing on samples at pH 4 versus control at pH 6, where LOQ stands for the limit of detection, NR refers to non-reduced conditions, R refers to reduced conditions, and NT means not tested.

TABLE 6

| | | | | | CE-SDS rel. area (%) | | | RP-HPLC rel. area (%) | | |
|---|---|---|---|---|---|---|---|---|---|---|
| | | SE-HPLC rel. area (%) | | | Main | LC | HC | Main | LC | HC |
| Sample | | Main | HMW | LMW | (NR) | (R) | (R) | (NR) | (R) | (R) |
| Control | T0 | 99.6 | 0.4 | <LOQ | 96.9 | 32.3 | 65.8 | 74.9 | 28.4 | 67.3 |
| (pH 6) | T_{A} | 99.7 | 0.4 | <LOQ | NT | NT | NT | 72.8 | 28.2 | 67.4 |
| | T_{B} | 99.6 | 0.4 | <LOQ | 96.8 | 32.0 | 66.1 | 73.1 | 28.1 | 67.1 |
| | T_{C} | 99.6 | 0.4 | <LOQ | 96.8 | 32.3 | 65.6 | 72.5 | 28.0 | 67.0 |
| Clipping | T0 | 59.7 | 40.4 | <LOQ | 96.9 | 32.2 | 65.8 | 73.0 | 28.4 | 67.6 |
| (pH 4) | T_{B} | 59.3 | 40.7 | <LOQ | 97.6 | 32.2 | 65.2 | 73.2 | 28.2 | 67.3 |
| | T_{C} | 58.3 | 41.7 | <LOQ | 96.5 | 32.3 | 64.1 | 71.7 | 27.9 | 66.2 |

Deamidation. The results of the deamidation study are reported in Table 7. Subjecting Antibody A to pH 8 resulted in the generation of acidic species in conjunction with a reduction of main species and basic species observed by icIEF, increasing from about 25% at T0 to about 50% after T_{C}. The control sample at pH 6 showed a more modest increase in acidic species, increasing from about 15% at T0 to about 25% after T_{C}.

Stressed samples at pH 8 also showed elevated levels of aggregates observed by SE-HPLC compared to the control, with about 1.6% aggregates present at T0 for the sample at pH 8 and about 0.4% aggregates present at T0 for the sample at pH 6. The level of aggregation decreased to about 1.4% after T_{C} in the sample at pH 8.

Table 7 summarizes the results of testing on samples at pH 8 versus control at pH 6, where LOQ stands for the limit of detection and NT means not tested.

TABLE 7

| | | SE-HPLC rel. area (%) | | | ieIEF rel. area (%) | | |
|---|---|---|---|---|---|---|---|
| Sample | | Main | HMW | LMW | Main | Acidic | Basic |
| Control | T0 | 99.6 | 0.4 | <LOQ | 60.1 | 16.8 | 23.1 |
| (pH 6) | T_{A} | 99.7 | 0.4 | <LOQ | NT | NT | NT |
| | T_{B} | 99.6 | 0.4 | <LOQ | 58.6 | 24.3 | 17.4 |
| | T_{C} | 99.6 | 0.4 | <LOQ | 55.0 | 27.6 | 17.4 |
| Deamidation | T0 | 98.4 | 1.6 | <LOQ | 56.8 | 26.3 | 16.9 |
| (pH 8) | T_{B} | 98.5 | 1.5 | <LOQ | 51.7 | 34.2 | 14.1 |
| | T_{C} | 98.6 | 1.4 | <LOQ | 40.1 | 49 | 10.9 |

Methionine oxidation. Table 8 reports the results of the methionine oxidation study. Solutions of Antibody A treated with hydrogen peroxide (H₂O₂) were evaluated by RP-HPLC and SE-HPLC. The data from RP-HPLC showed a clear change of the peak areas together with a retention time shift of the stressed-sample profiles in non-reduced RP-HPLC, and these changes were more pronounced in the high-stress sample. As observed by non-reduced RP-HPLC, the stressed samples showed an increase in the relative area of the main peak. Reduced RP-HPLC revealed even more distinct changes in the profiles of LC and HC after stress application. Data from SE-HPLC showed minor aggregation (generating about 1% aggregates under high stress conditions), but no relevant differences with regard to fragmentation caused by the modifications.

Table 8 summarizes the results of testing on samples incubated with H₂O₂ versus control, where LOQ stands for the limit of detection, NR refers to non-reduced conditions, R refers to reduced conditions, and NT means not tested.

TABLE 8

| | | | | | RP-HPLC rel. area (%) | | |
|---|---|---|---|---|---|---|---|
| | | SE-HPLC rel. area (%) | | | Main | LC | HC |
| Sample | | Main | HMW | LMW | (NR) | (R) | (R) |
| Control | T0 | 99.6 | 0.4 | <LOQ | 74.9 | 28.4 | 67.3 |
| (pH 6) | T_{A} | 99.7 | 0.4 | <LOQ | 72.8 | 28.2 | 67.4 |
| | T_{B} | 99.6 | 0.4 | <LOQ | 73.1 | 28.1 | 67.1 |
| | T_{C} | 99.6 | 0.4 | <LOQ | 72.5 | 28.0 | 67.0 |
| Oxidation | H₂O₂ | 99.4 | 0.6 | <LOQ | 83.4 | 11.8 | 9.8 |
| | High | | | | | | |
| | Stress | | | | | | |
| | H₂O₂ | 98.7 | 1.3 | <LOQ | 85.0 | 0.7 | 2.3 |
| | Low | | | | | | |
| | Stress | | | | | | |

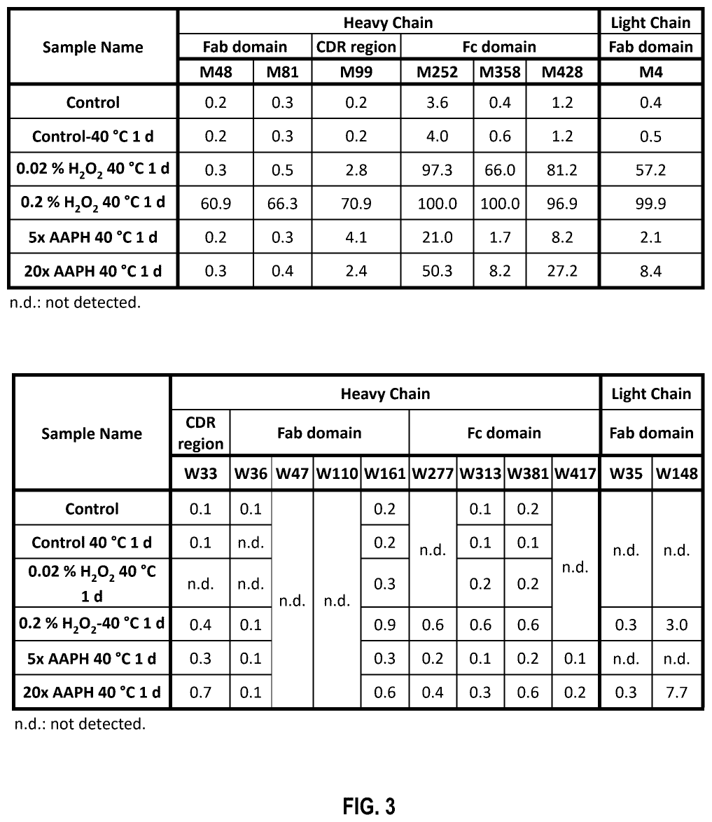

A peptide map analysis of the H₂O₂-stress samples was performed to determine the extent of oxidation of the methionine and tryptophan residues. FIG. 3 provides the results of peptide mapping by percent oxidation at each oxidation site in the heavy chain and light chain identified after H₂O₂ and AAPH incubation, where “n.d.” means not detected. Table 9 provides a summary of the results in FIG. 3 for the percent oxidation at each oxidation site in the heavy chain and light chain identified after H₂O₂ incubation, where “n.d.” means not detected.

TABLE 9

| | | | Control | Low Stress | High Stress |
|---|---|---|---|---|---|
| Sample | | Control | 40° C., T_{A} | H₂O₂, T_{A} | H₂O₂, T_{A} |
| HC Fab | M48 | 0.2 | 0.2 | 0.3 | 60.9 |
| HC Fab | M81 | 0.3 | 0.3 | 0.5 | 66.3 |
| HC Fab/ | M99 | 0.2 | 0.2 | 2.8 | 70.9 |
| CDR | | | | | |
| HC Fc | M252 | 3.6 | 4.0 | 97.3 | 100.0 |
| HC Fc | M358 | 0.4 | 0.6 | 66.0 | 100.0 |
| HC Fc | M428 | 1.2 | 1.2 | 81.2 | 96.9 |
| LC Fab | M4 | 0.4 | 0.5 | 57.2 | 99.9 |
| HC Fab/ | W33 | 0.1 | 0.1 | n.d. | 0.4 |
| CDR | | | | | |
| HC Fab | W36 | 0.1 | n.d. | n.d. | 0.1 |
| HC Fab | W47 | n.d. | n.d. | n.d. | n.d. |
| HC Fab | W110 | n.d. | n.d. | n.d. | n.d. |
| HC Fab | W161 | 0.2 | 0.2 | 0.3 | 0.9 |
| HC Fc | W277 | n.d. | n.d. | n.d. | 0.6 |
| HC Fc | W313 | 0.1 | 0.1 | 0.2 | 0.6 |
| HC Fc | W381 | 0.2 | 0.1 | 0.2 | 0.6 |
| HC Fc | W417 | n.d. | n.d. | n.d. | n.d. |
| LC Fab | W35 | n.d. | n.d. | n.d. | 0.3 |
| LC Fab | W148 | n.d. | n.d. | n.d. | 3.0 |

Oxidation occurred in 96.9% to 100% of the methionine-residues in the Fc domain of Antibody A (M256, M358, M428) under high stress conditions (high-stress H₂O₂ at 40° C. at T_{A}). The susceptibility of such conserved methionine-residues is known; an impact on activity or binding of Antibody A is unknown. Oxidation also occurred in 60.9% to 70.9% of the methionine-residues in the Fab domain (HC M48, M81, M99 and LC M4) under high stress conditions, with 70.9% of residue M99, which is located in the CDR, undergoing oxidation. The use of H₂O₂ as oxidizing agent shows a good selectivity for methionine-residues, as tryptophan residues were not significantly oxidized (except for W148, which underwent significant oxidation when also treated with AAPH, as discussed below).

Tryptophan oxidation. The tryptophan oxidation results are reported in Table 10. Solutions of Antibody A treated with AAPH showed a clear change in the RP-HPLC (non-reduced as well as reduced) profile with an increase of the post main peak species when compared with the control samples, indicating oxidation-induced changes in the polarity of the molecule. SE-HPLC data showed that samples treated with low-stress AAPH contained about 6% aggregates, which increased to about 20% in the high-stress AAPH sample.

Table 10 summarizes the results of testing on samples incubated with AAPH versus control, where LOQ stands for the limit of detection, NR refers to non-reduced conditions, R refers to reduced conditions, and NT means not tested.

TABLE 10

| | | | | | RP-HPLC rel. area (%) | | |
|---|---|---|---|---|---|---|---|
| | | SE-HPLC rel. area (%) | | | Main | LC | HC |
| Sample | | Main | HMW | LMW | (NR) | (R) | (R) |
| Control | T0 | 99.6 | 0.4 | <LOQ | 74.9 | 28.4 | 67.3 |
| (pH 6) | T_{A} | 99.7 | 0.4 | <LOQ | 72.8 | 28.2 | 67.4 |
| | T_{B} | 99.6 | 0.4 | <LOQ | 73.1 | 28.1 | 67.1 |
| | T_{C} | 99.6 | 0.4 | <LOQ | 72.5 | 28.0 | 67.0 |
| Oxidation | AAPH | 93.2 | 6.8 | <LOQ | 74.9 | 28.0 | 62.3 |
| | Low | | | | | | |
| | Stress | | | | | | |
| | AAPH | 80.3 | 19.5 | 0.1 | 67.5 | 0.4 | 26.9 |
| | High | | | | | | |
| | Stress | | | | | | |

A peptide map analysis of the AAPH-stress samples was performed to determine the extent of oxidation of the methionine and tryptophan residues. FIG. 3 provides the results of peptide mapping by percent oxidation at each oxidation site in the heavy chain and light chain identified after H₂O₂ and AAPH incubation, where “n.d.” means not detected. Table 11 provides a summary of the results in FIG. 3 for the percent oxidation at each oxidation site in the heavy chain and light chain identified after AAPH incubation, where “n.d.” means not detected.

TABLE 11

| | | | | Low Stress | High Stress |
|---|---|---|---|---|---|
| | | | Control | AAPH, | AAPH, |
| Sample | | Control | 40° C., T_{A} | 40° C., T_{A} | 40° C., T_{A} |
| HC Fab | M48 | 0.2 | 0.2 | 0.2 | 0.3 |
| HC Fab | M81 | 0.3 | 0.3 | 0.3 | 0.4 |
| HC Fab/ | M99 | 0.2 | 0.2 | 4.1 | 2.4 |
| CDR | | | | | |
| HC Fc | M252 | 3.6 | 4.0 | 21.0 | 50.3 |
| HC Fc | M358 | 0.4 | 0.6 | 1.7 | 8.2 |
| HC Fc | M428 | 1.2 | 1.2 | 8.2 | 27.2 |
| LC Fab | M4 | 0.4 | 0.5 | 2.1 | 8.4 |
| HC Fab/ | W33 | 0.1 | 0.1 | 0.3 | 0.7 |
| CDR | | | | | |
| HC Fab | W36 | 0.1 | n.d. | 0.1 | 0.1 |
| HC Fab | W47 | n.d. | n.d. | n.d. | n.d. |
| HC Fab | W110 | n.d. | n.d. | n.d. | n.d. |
| HC Fab | W161 | 0.2 | 0.2 | 0.3 | 0.6 |
| HC Fc | W277 | n.d. | n.d. | 0.2 | 0.4 |
| HC Fc | W313 | 0.1 | 0.1 | 0.1 | 0.3 |
| HC Fc | W381 | 0.2 | 0.1 | 0.2 | 0.6 |
| HC Fc | W417 | n.d. | n.d. | 0.1 | 0.2 |
| LC Fab | W35 | n.d. | n.d. | n.d. | 0.3 |
| LC Fab | W148 | n.d. | n.d. | n.d. | 7.7 |

The results show that, with one exception, the tryptophan residues in the AAPH-stressed samples were not significantly oxidized. Only the tryptophan at position 148 of the light chain showed significant oxidation (7.7%) after AAPH stress. Low- and high-stress AAPH induced significant oxidation levels of methionine-residues in the Fc region of the heavy chain, i.e. M252, M358 and M428, which are often conserved, solvent-exposed, and prone to oxidation.

### Example 4: Conformational and Colloidal Stability of Antibody A

The conformational and colloidal stability of Antibody A was assessed under eight buffering conditions with varying pH and ionic strength.

The Stock Solution of Antibody A was buffer exchanged into eight different solutions with varying buffer substances, pH, and ionic strengths using spin filtration, as summarized in Table 12. Buffer exchange was verified by osmolality and pH measurements. A solution of Antibody A was prepared and filtered through 0.2 μm filters into clean reaction tubes.

TABLE 12

| | | +100 mM | +100 mM |
|---|---|---|---|
| Buffer 10 mM/pH | No Salt | NaCl | Arginine-HCL |
| Histidine-HCl/pH 5 | X | X | X |
| Histidine-HCl/pH 6 | X | X | — |
| Na-phosphate/pH 7 | X | X | X |

A dilution series of protein concentrations was prepared from the two filtered solutions (buffer and protein solution from buffer exchange), which was then transferred in triplicates into a 384 well plate.

Conformational Stability. Conformational stability was evaluated by measuring the temperatures at the onset of unfolding (T_{unfold}) and aggregation (T_{Agg}) of Antibody A for all eight solutions in the buffers identified in Table 12.

Differential scanning fluorescence (DSF) measurements were collected using a thermal ramp (20-95° C.). Proteins unfold under thermal stress, gradually exposing buried tryptophan-residues to the solvent, which leads to an increase of the fluorescence signal when excited with laser light at 266 nm. The apparent first unfolding temperature of the protein (T_{unfold}) was calculated from the acquired fluorescence signal.

The unfolding temperatures (T_{unfold}) ranged from 54.7° C. to 67.4° C. for the eight different buffer conditions (Table 12). The lowest T_{unfold} were observed at pH 5, an increase in pH led to an increase in T_{unfold}. The addition of NaCl and Arg-HCl both led to a minor decrease of T_{unfold}. Overall, the data suggested good thermal stability for all samples at pH 6-7 with or without excipient addition, as summarized in Table 13.

Next, static light scattering signals on the eight buffer solutions were recorded with the same device, which was then used to determine the onset temperature of aggregation (T_{Agg}).

The aggregation onset temperatures (T_{Agg}) ranged from 51.8° C. to 74.9° C., as summarized in Table 13. For the samples without excipient addition T_{Agg} was lowest at pH 5 and highest at pH 6, while the sample at pH 7 showed an intermediate value. Addition of NaCl and addition of Arg-HCl resulted in an increase of T_{Agg} at pH 5 and pH 7, but a slight decrease of T_{Agg} at pH 6. Arg-HCl addition showed a slightly higher improvement of T_{Agg} than NaCl addition.

TABLE 13

| | | 100 mM | 100 mM | T_{unfold} | T_{Agg} |
|---|---|---|---|---|---|
| Buffer (10 mM) | pH | NaCl | Arg-HCl | in ° C. | in ° C. |
| His-HCl | 5 | — | — | 59.3 | 51.8 |
| His-HCl | 6 | — | — | 65.6 | 74.9 |
| Na-phosphate | 7 | — | — | 68.0 | 65.5 |
| His-HCl | 5 | X | — | 54.7 | 59.7 |
| His-HCl | 6 | X | — | 64.3 | 67.7 |
| Na-phosphate | 7 | X | — | 67.4 | 70.7 |
| His-HCl | 5 | — | X | 56.4 | 70.0 |
| Na-phosphate | 7 | — | X | 66.2 | 73.4 |

Antibody A exhibited superior conformational stability at a pH around 6-7 versus pH 5. The addition of NaCl and Arg-HCl had a minor impact on the thermal stability of Antibody A according to DSF measurements of T_{unfold}. SLS measurements revealed the highest T_{Agg} at pH 6 in the absence of NaCl and Arg-HCl and at pH 7 with the addition of Arg-HCl. All samples at pH 5 showed noticeably lower T_{Agg} values than samples at pH 6-7.

Colloidal Stability. Colloidal stability was evaluated by measuring the 2nd virial coefficient (A2) and the diffusion interaction parameter (kD) of Antibody A for all eight solutions in Table 12. The diffusion interaction parameter kD was calculated from dynamic light scattering (DLS) data, while the second osmotic virial coefficient A2 was calculated from the obtained static light scattering (SLS) data.

A2 provides an assessment of how a solute such as Antibody A behaves in solution, specifically whether attractive or repulsive interactions exist. A positive A2 value indicates intermolecular repulsion, while a negative A2 value indicates intermolecular attraction, which is not desired for colloidal stability. Higher A2 values correspond to increased colloidal stability.

A2 values for Antibody A in the eight solutions of Table 12 indicated weak intermolecular forces. In the absence of excipients, weak attractive interactions were measured at pH 6 and 7, while weak repulsive interactions were measured at pH 5. The addition of NaCl had a positive effect on A2 values measured at pH 6-7, while the addition of Arg-HCl had a positive effect on A2 at all pH values tested by slightly increasing the repulsive interactions. These data are summarized in Table 14.

The first-order diffusion parameter (kD) also provides an indication of the intermolecular interactions and is closely linked to A2. kD values for Antibody A in the eight solutions of Table 12 showed a comparable trend to A2 values, with Antibody A exhibiting stronger repulsive interactions at pH 5 than at pH 6 and 7. The addition of NaCl or Arg-HCl increased repulsive interactions under all pH conditions. The increase in repulsive interactions were more pronounced for the addition of Arg-HCl, as summarized in Table 14.

TABLE 14

| | | 100 mM | 100 mM | kD in | A2 in |
|---|---|---|---|---|---|
| Buffer (10 mM) | pH | NaCl | Arg-HCl | ml/g | (10⁻⁵*ml*mol)/g² |
| His-HCl | 5 | — | — | −7.4 | 2.0 |
| His-HCl | 6 | — | — | −13.3 | −1.5 |
| Na-phosphate | 7 | — | — | −10.8 | −0.7 |
| His-HCl | 5 | X | — | −3.3 | 1.2 |
| His-HCl | 6 | X | — | 0.1 | 2.3 |
| Na-phosphate | 7 | X | — | −1.9 | 2.3 |
| His-HCl | 5 | — | X | −0.7 | 4.4 |
| Na-phosphate | 7 | — | X | 0.9 | 5.5 |

Evaluation of the A2 and kD values for solutions of Antibody A overall showed weak intermolecular forces. At solution conditions without NaCl or Arg-HCl, stronger repulsive interactions were measured at pH 5 versus pH 6 or 7. Addition of NaCl and Arg-HCl both improved the colloidal stability of Antibody A at pH 6 and pH 7 according to kD and A2 data, and this was more pronounced for the addition of Arg-HCl.

### Example 5: Formulation Screening

Antibody A was manufactured and supplied in a buffer of 10 mM sodium citrate with 150 mM NaCl at approximately pH 6.7 and at a concentration of approximately 60 mg/mL (formulation F4).

Formulations F1-F3 comprising Antibody A were prepared as summarized in Table 15 with the goal of developing a stable, high concentration, liquid formulation suitable for either subcutaneous injection or for intravenous infusion. Formulation F4 was used as a comparative formulation.

TABLE 15

| | Antibody A | | | | | | |
|---|---|---|---|---|---|---|---|
| No. | (mg/mL) | Buffer | pH | Excipient 1 | Excipient 2 | Excipient 3 | Surfactant |
| F1 | 150 | 20 mM | 6.2 | 115 mM | 10 mM | — | PS80 |
| | | His/HisHCl | | NaCl | methionine | | (0.04% w/v) |
| F2 | 150 | 20 mM | 6.2 | 50 mM | — | 125 mM | PS80 |
| | | His/HisHCl | | NaCl | | sucrose | (0.04% w/v) |
| F3 | 150 | 20 mM | 6.2 | 50 mM | 10 mM | 110 mM | PS80 |
| | | His/HisHCl | | arginine | methionine | sucrose | (0.04% w/v) |
| | | | | HCl | | | |
| F4 | 60 | 10 mM | 6.7 | 150 mM | — | — | PS80 |
| | | Na-citrate | | NaCl | | | (0.01% w/v) |

Formulations F1-F3 were manufactured by subjecting formulation no. F4 to (1) buffer exchange to achieve target buffer concentration and pH by tangential flow filtration followed by (2) the up-concentration above the target concentration by tangential flow filtration (F2 and F3) or by centrifugation in ultrafiltration centrifugal devices (F1, due to lower volume processing).

The protein concentration, pH value and density were determined upon processing of Antibody A and utilized for the required calculation of batch composition of each formulation by using standard dilution procedure.

All bulk formulation solutions were filtered using a 0.22 μm polyvinylidene fluoride (PVDF) membrane filter. The protein concentration, osmolality, and pH of the finally compounded solutions were determined, and the results are summarized in Table 16. For F1-F4, the pH values, osmolality values, and protein concentration values were taken over from T0 results. For F4, the pH of 7.0 was higher than target at T0. The deviation was not confirmed at further timepoints (pH 6.8).

TABLE 16

Formulation

| Test | F1 | F2 | F3 | F4 |
|---|---|---|---|---|
| pH | 6.6 | 6.3 | 6.2 | 7.0 |
| Osmolality by freezing | 284 | 332 | 293 | 302 |
| point [mOsm/kg] | | | | |
| Protein concentration | 152.0 | 153.7 | 154.9 | 60.4 |
| [mg/mL] | | | | |
| Viscosity at 25° C. [mPa · s] | 7 | 10 | 8 | 2 |

The primary packaging materials were prepared as appropriate and each formulation filled manually, observing aseptic techniques, into 6R/20 mm glass type I vials at a target fill volume of 2.4 mL, stoppered with 20 mm bromobutyl rubber stoppers and sealed with 20 mm aluminum flip-off seals. Samples of all formulations were labelled and stored at 5±3° C. till distribution for the stability studies.

Testing samples of each formulation were distributed in upright position into the stability chambers at designated temperatures of 5±3° C., 25±2° C. and 60%±5% relative humidity (RH), or 40° C.±2° C. and 75%±5% relative humidity. Samples were tested at the timepoints identified in Table 17 according to test methods specified in Table 18.

One testing sample per liquid formulation was subjected to shaking stress. Samples were tested according to test methods shown in the study program in Table 18.

A testing sample per liquid formulation was subjected in vertical position to four freeze/thaw cycles from −65° C. or below to room temperature. Samples were tested according to test methods shown in the study program in Table 18.

Table 17 summarizes the conditions and timepoints at which 25 vials of each formulation were tested.

TABLE 17

| | | | 25 ± 2° C./ | 40° C. ± 2° C./ |
|---|---|---|---|---|
| | Initial/ | | 60% ± 5% | 75% ± 5% |
| Time of Testing | T0 | 5 ± 3° C. | RH | RH |
| Initial/T0 | 1 | | | |
| Freeze thaw from | 1 | | | |
| 25° C. to −65° C. | | | | |
| Shaking stress | | | 1 | |
| 2 weeks | | | 1 | 1 |
| 4 weeks | | 1 | 1 | 1 |
| 8 weeks | | 1 | 1 | 1 |
| 3 months | | 1 | 1 | 1 |
| 6 months | | 1 | 1 | |
| 9 months | | 1 | | |
| 12 months | | 1 | | |
| Reserve¹ | | 3 | 2 | 2 |
| Vials/temp | 2 | 9 | 8 | 6 |

| | |
|---|---|
| Total vials | 25 |

The stability study was extended to 24 months by using reserve vials of F3 and F4 stored at 5° C. At initial and subsequent time-points, testing samples of each formulation were tested according to test methods shown in Table 18.

TABLE 18

| | Dosage | Stability |
|---|---|---|
| Analytical Method | Form | Timepoint |
| Visible Particles (Black & White) | Liquid | All |
| Clarity & opalescence of a solution | Liquid | All |
| (turbidimetry) | | |
| Color measurement using colorimeter | Liquid | All |
| Sub-visible particles by Light Obscuration | Liquid | All |
| (LO), low volume method | | |
| Determination of pH | Liquid | All |
| Osmolality by Freezing Point Depression | Liquid | T0 |
| Protein content by SoloVPE ® | Liquid | All |
| Purity by Size Exclusion-HPLC (SE-HPLC) | Liquid | All |
| Purity by Chip-based CE-SDS (non-reducing | Liquid | All |
| and reducing conditions) | | |
| Purity by Imaged Capillary Isoelectric Focusing | Liquid | All except |
| (icIEF) using ICE3 | | freeze-thaw |
| | | and shaking |
| Determination of Polysorbate 20/80 Content by | Liquid | All |
| Fluorescent Micelle Assay (HPLC) | | |
| Purity by reverse phase-HPLC (RP-HPLC) | Liquid | All |
| (reduced and nonreduced condition) | | |

### Results

Freeze/thaw cycles. All formulations subjected to 4 freeze-thaw cycles (−65° C. to room temperature) did not show any relevant changes in any of the analytical methods compared to the initial non-stressed samples, indicating that formulations effectively stabilized Antibody A against freeze-thaw stress.

Agitation. Shaking stress at ambient temperature led to an increase in visible particles for F1-F3, with more visible particles observed in F3 than either F1 or F2. F4, which contained the lowest concentration of protein, remained free of visible particles. However, F4 appeared the most unstable regarding aggregate formation and loss of main peak by SE-HPLC after agitation. No changes in purity were observed for other formulations.

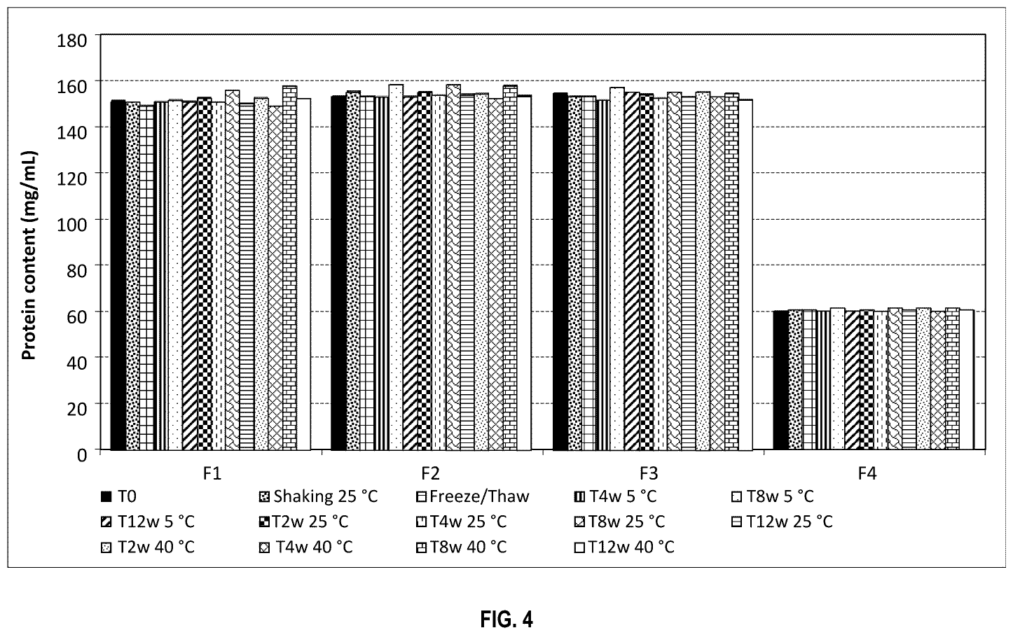

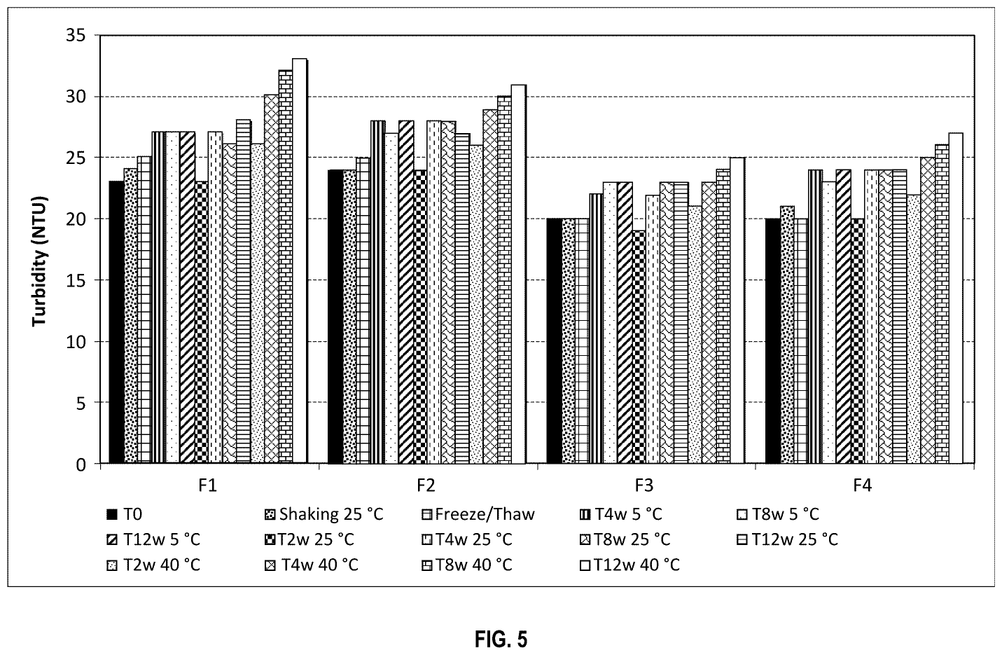

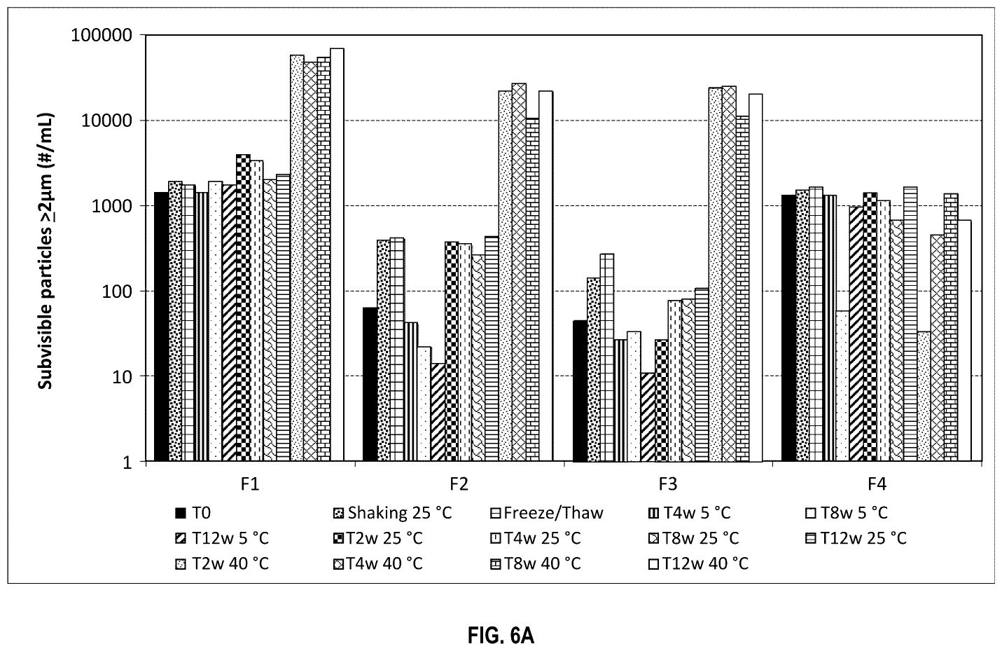

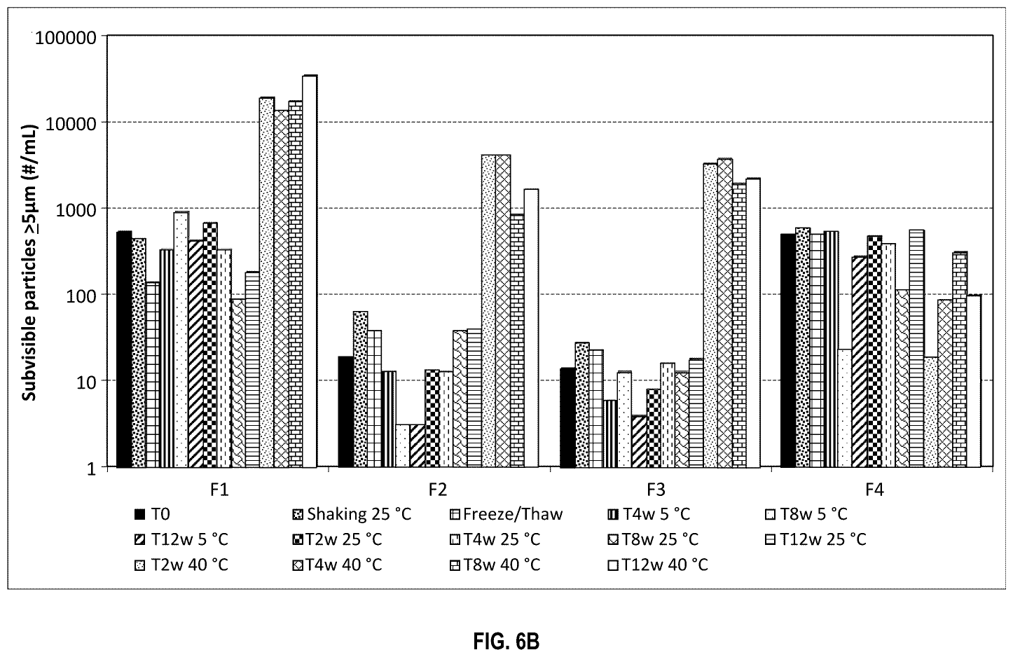

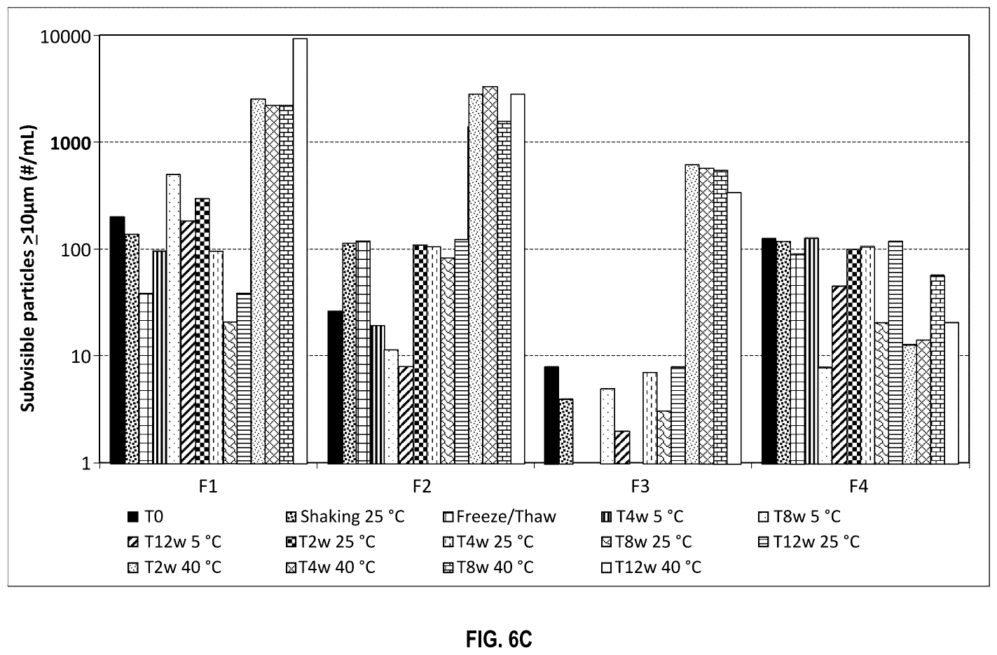

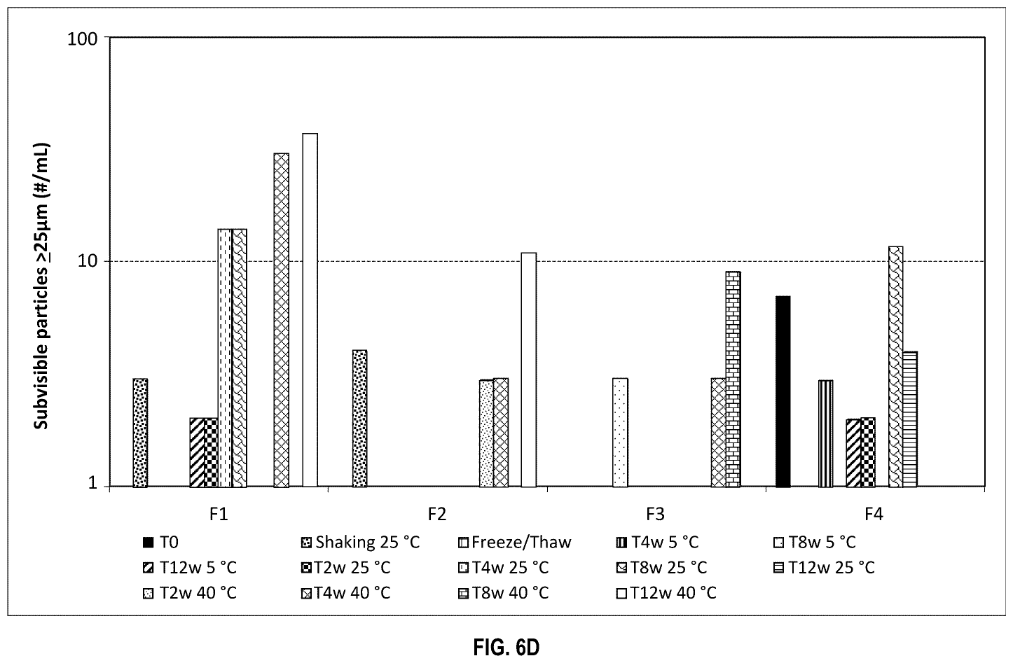

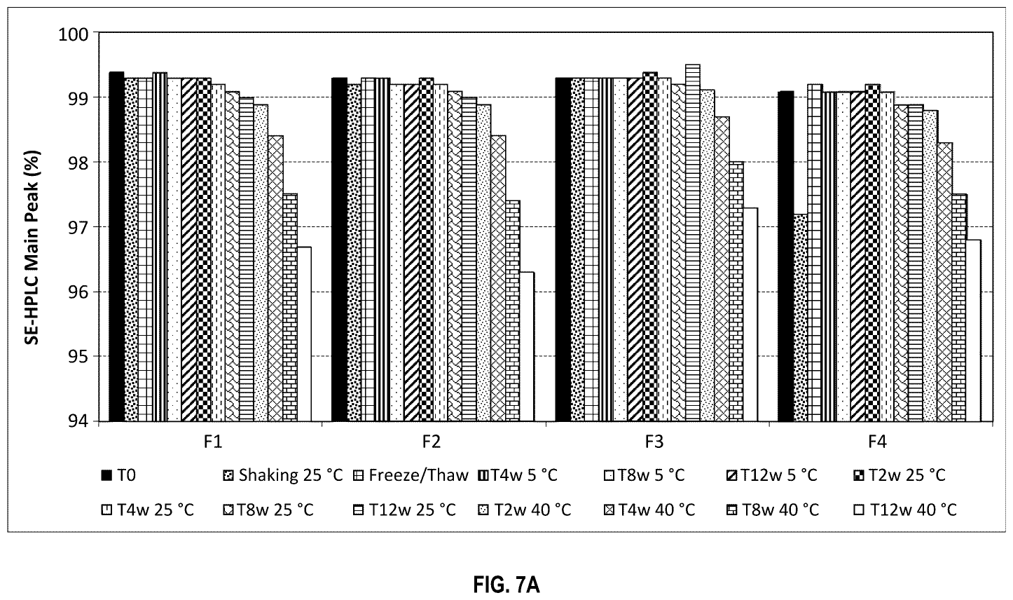

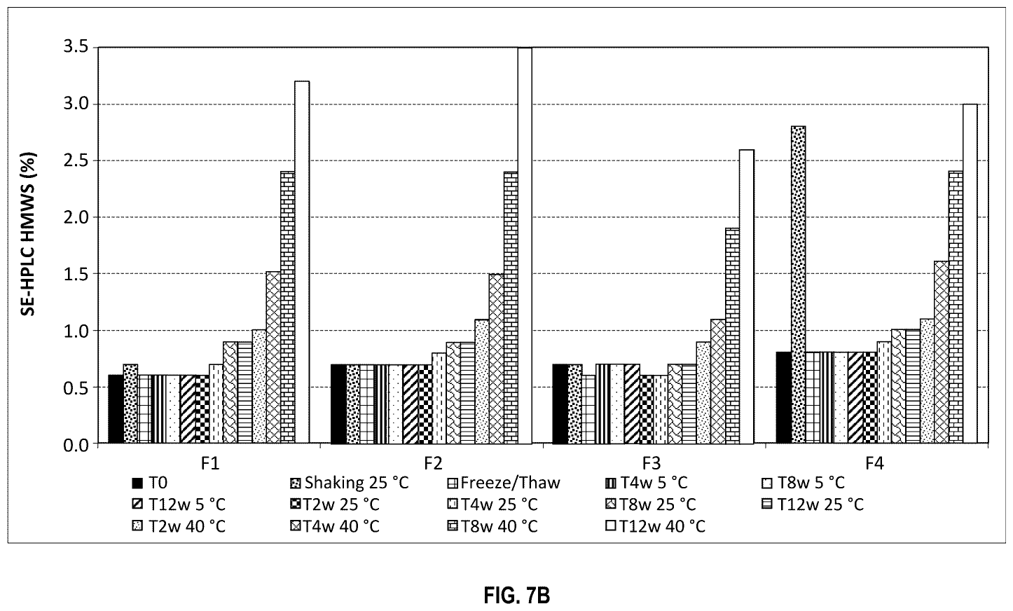

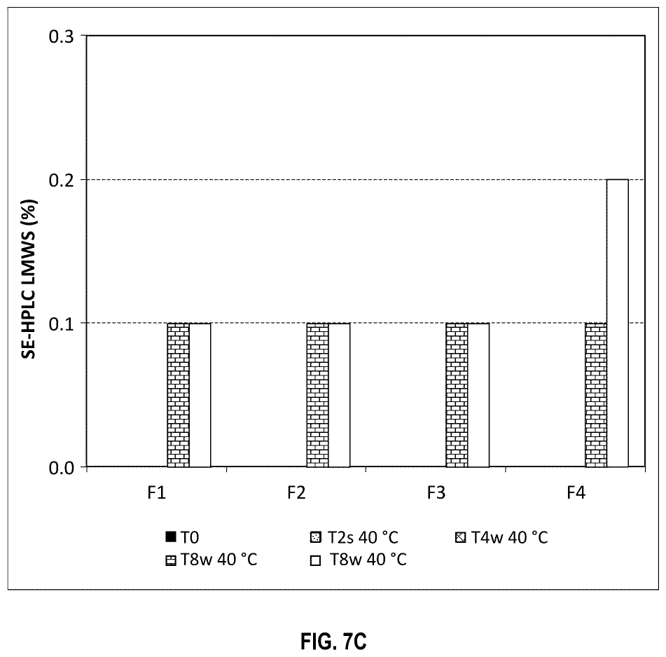

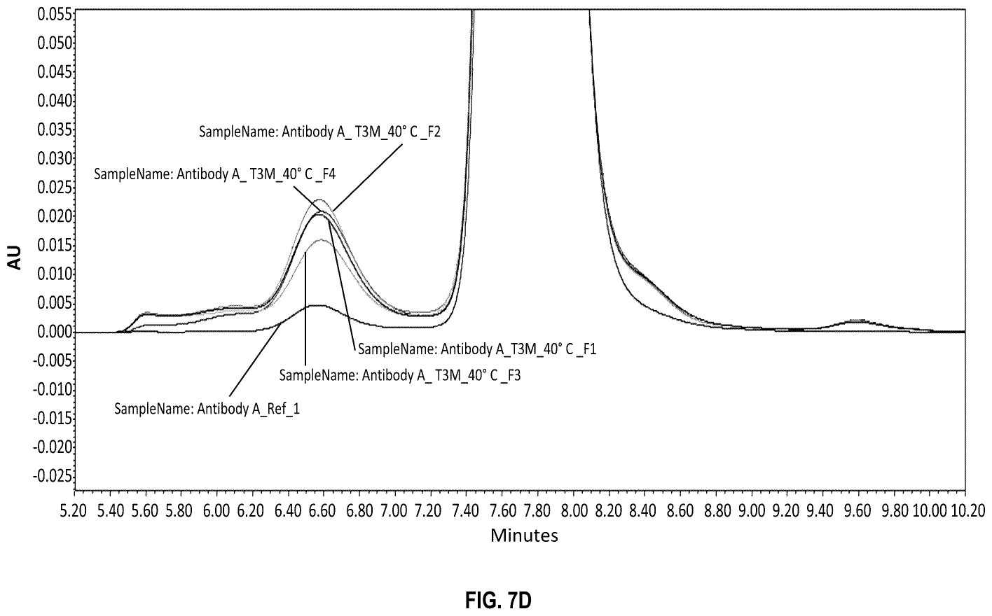

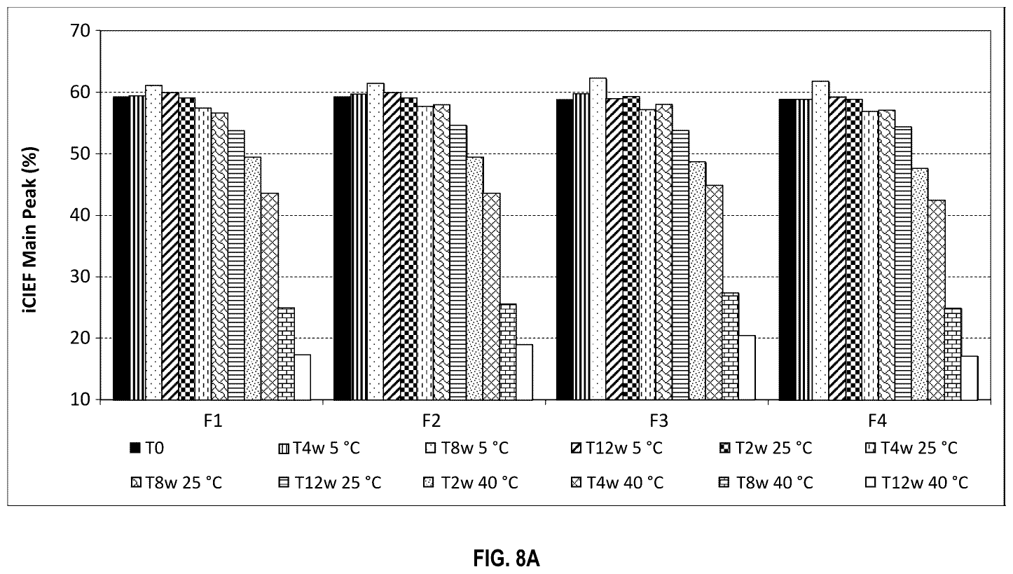

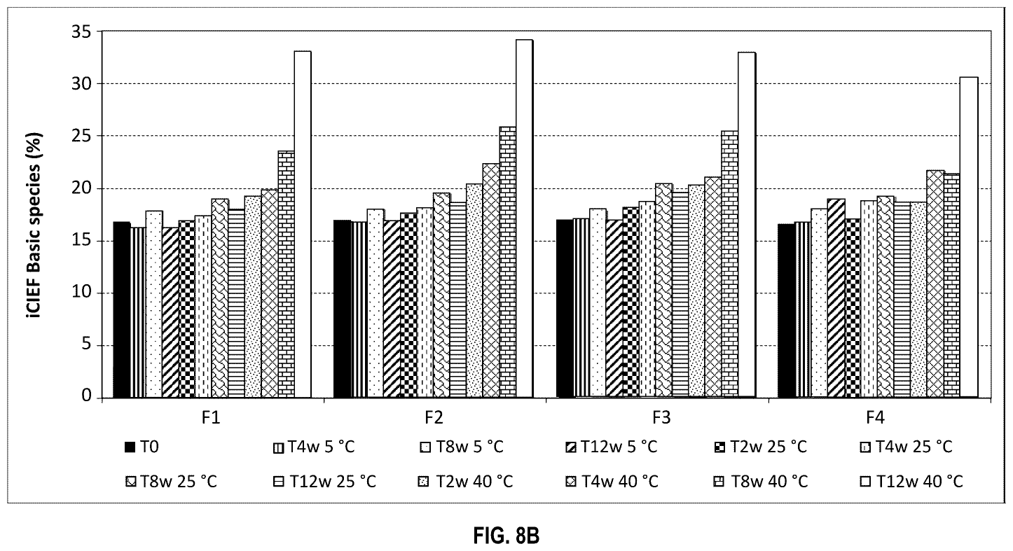

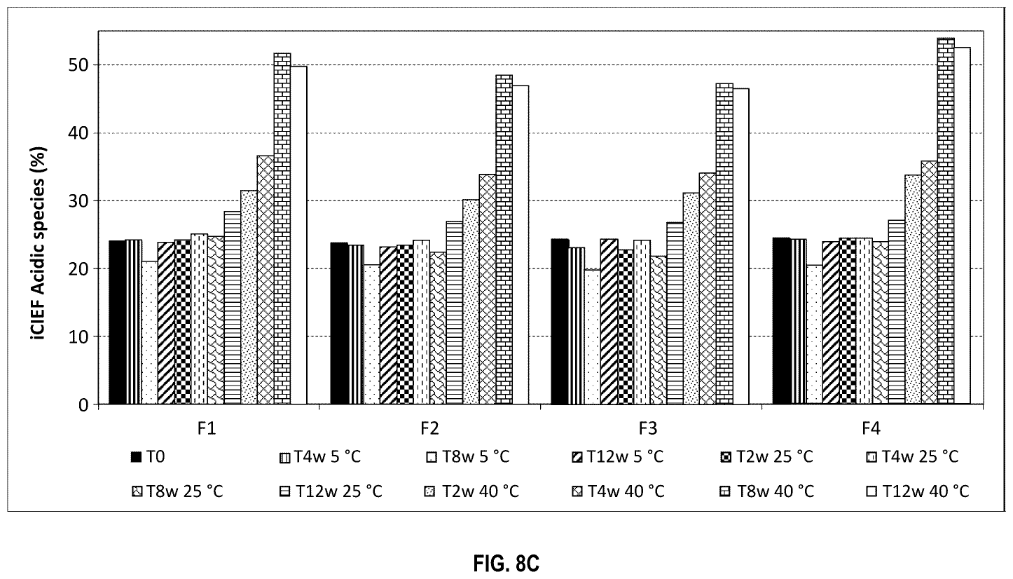

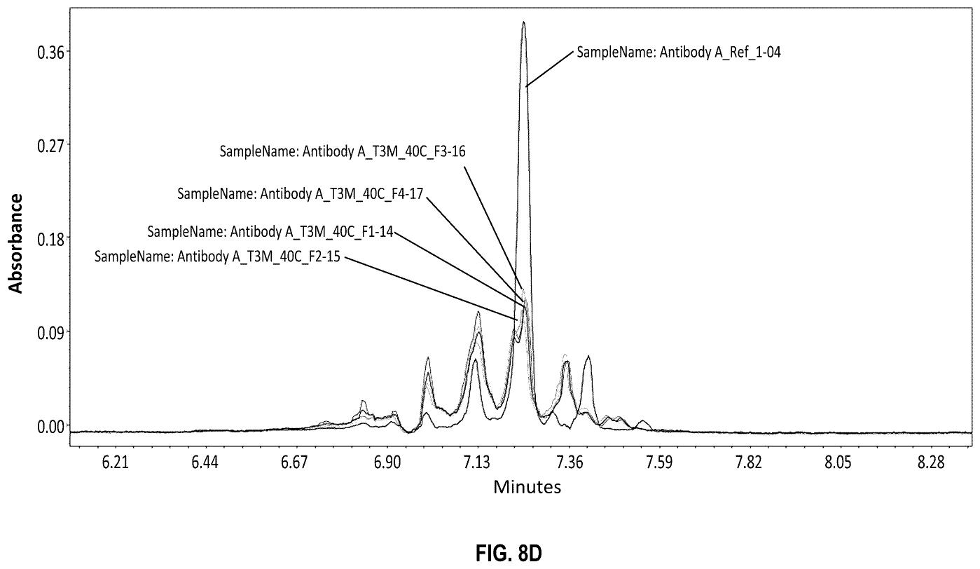

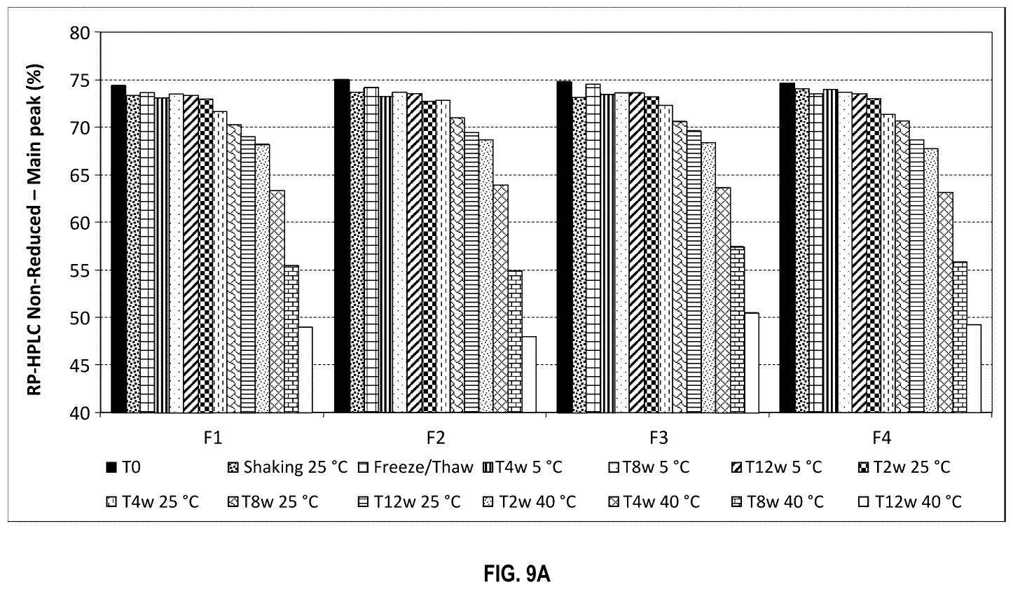

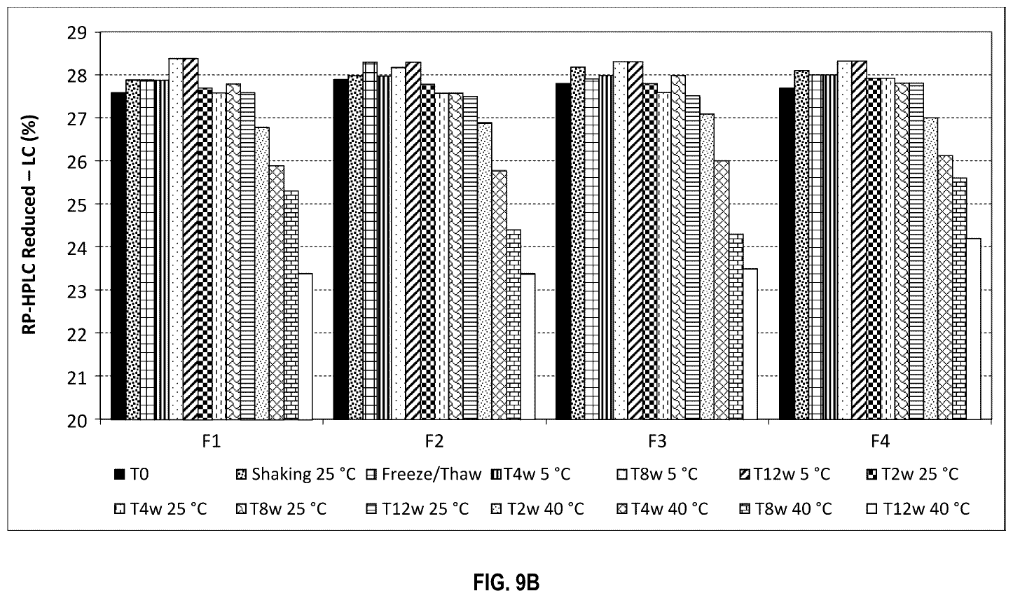

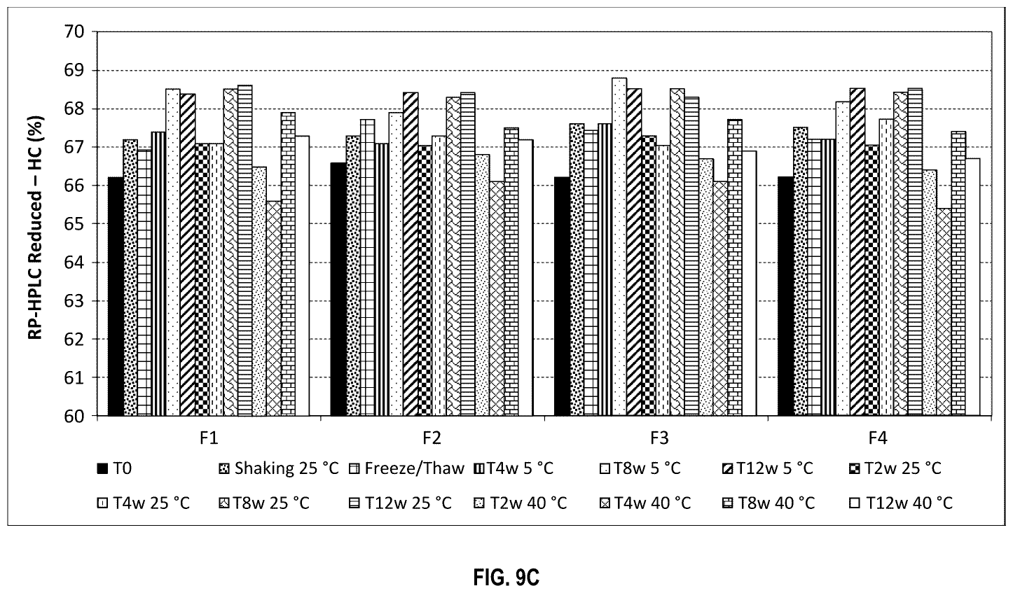

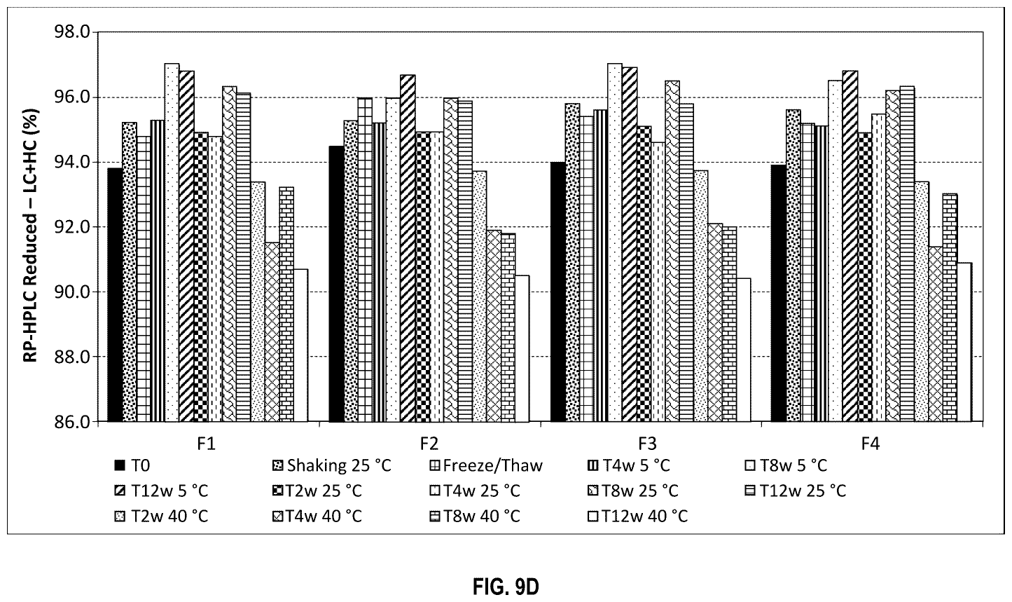

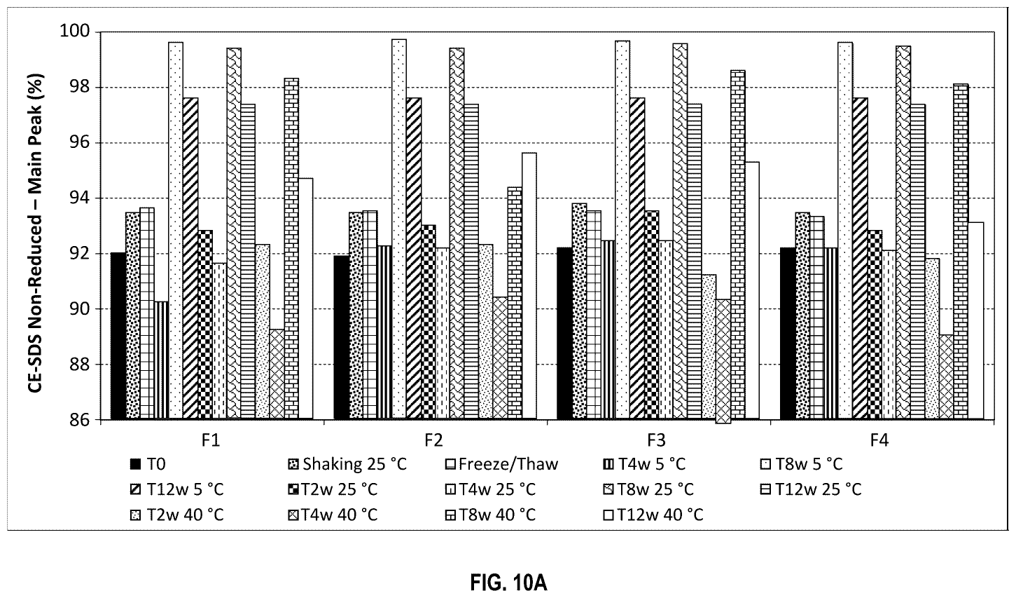

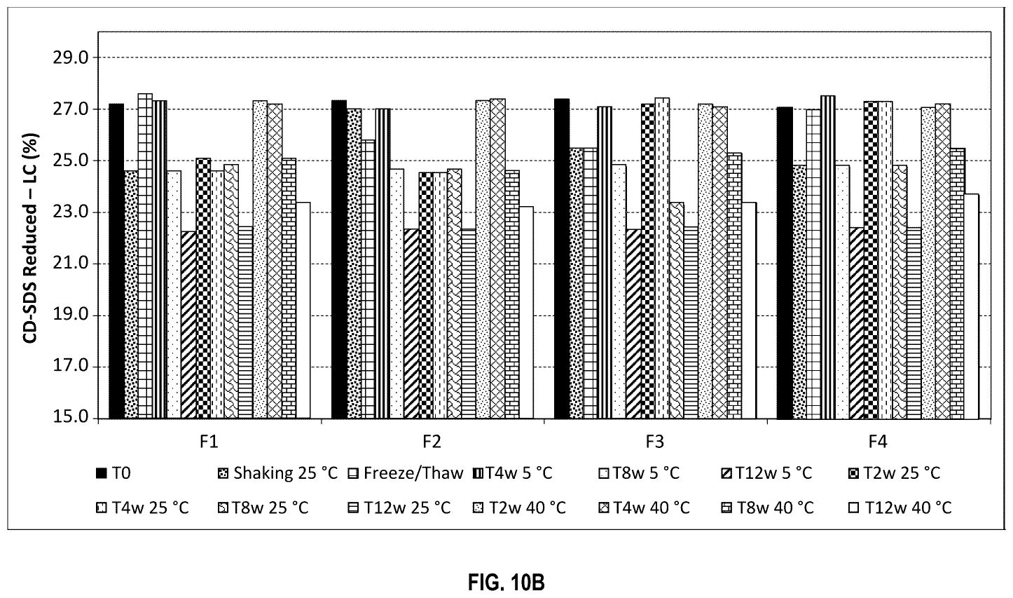

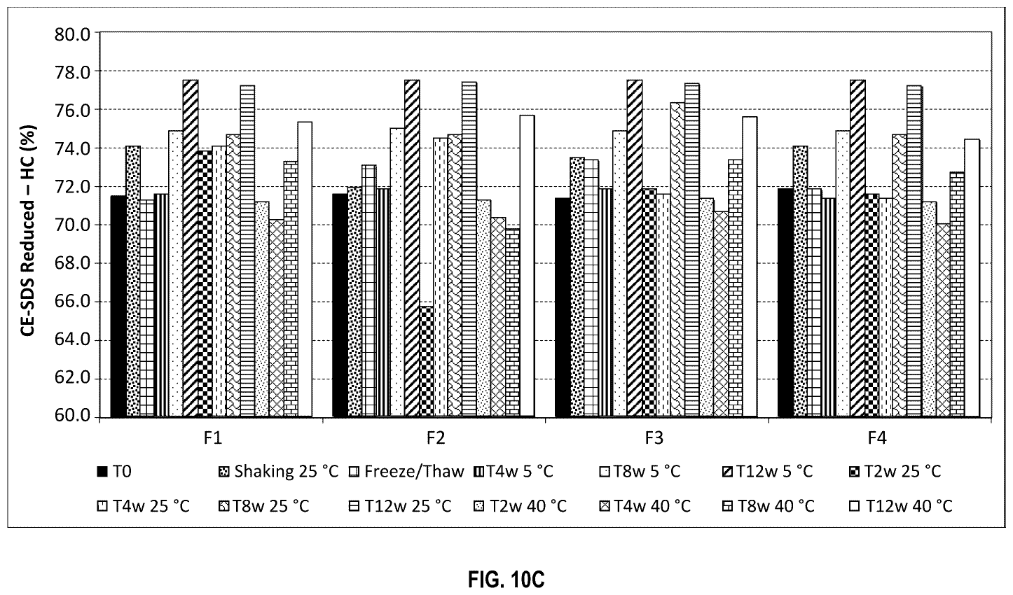

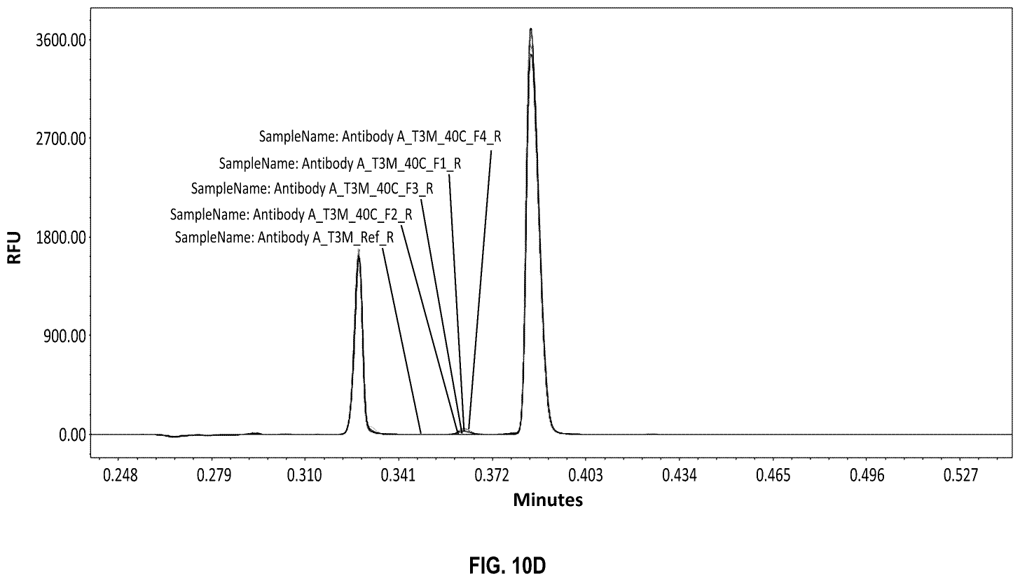

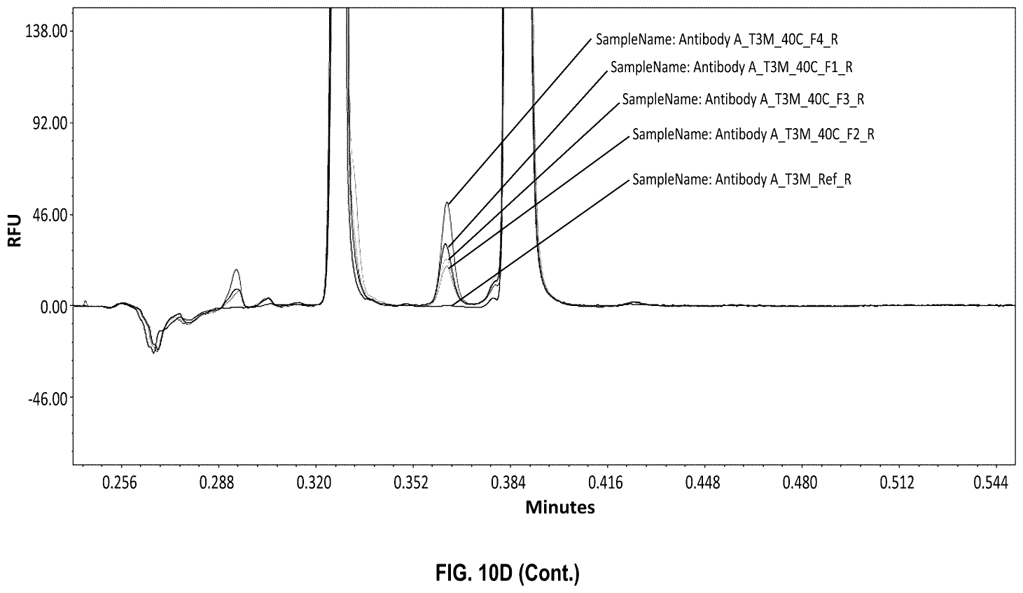

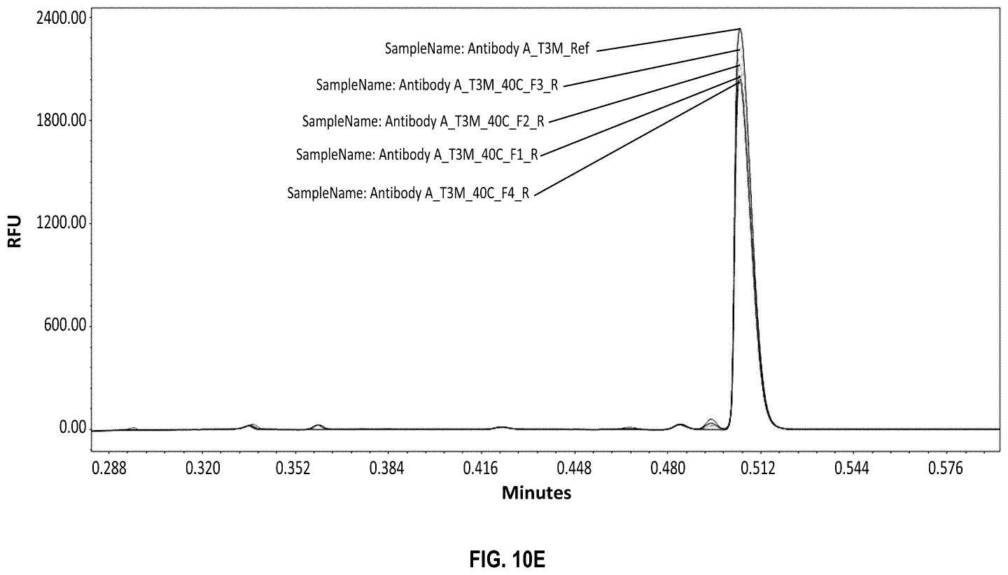

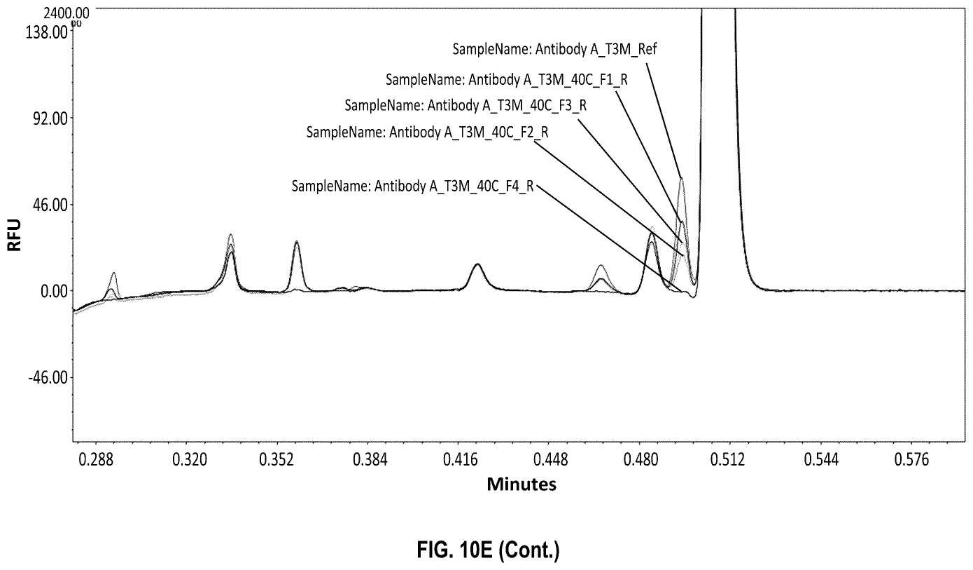

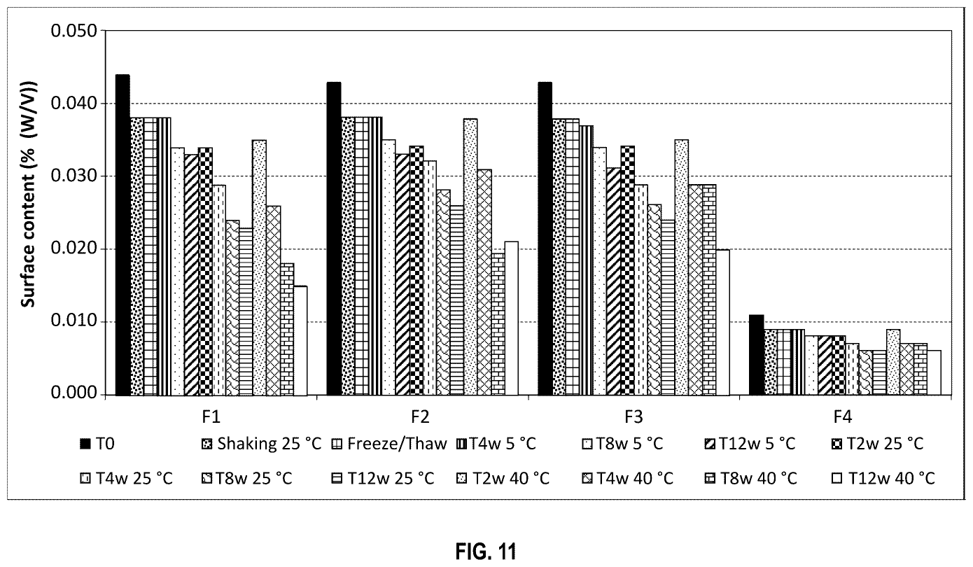

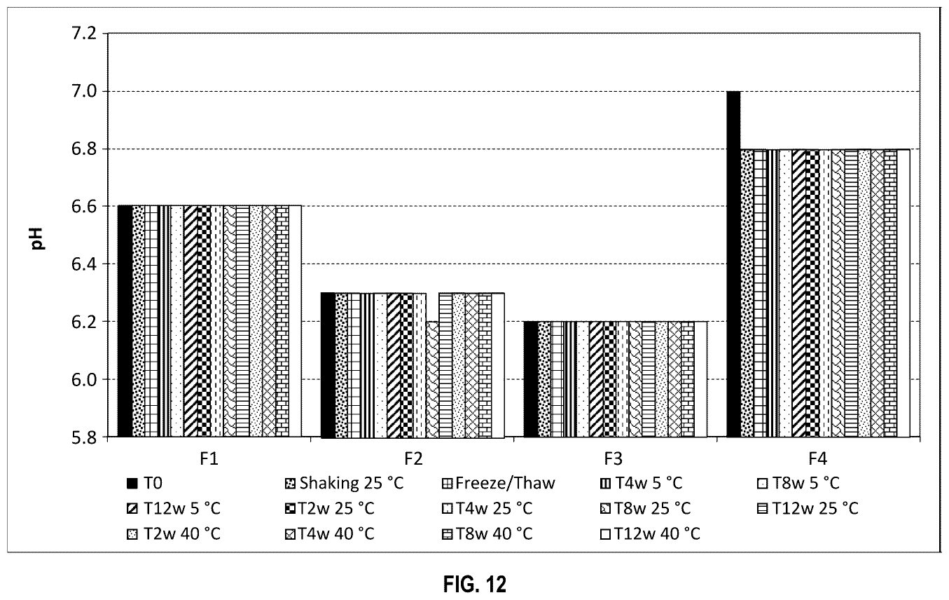

Short-term stability: T0 to 12 weeks. Results of short-term stability studies for F1-F4 upon storage for up to 12 weeks are shown in FIGS. 4-12. FIG. 4 shows the protein content by UV/Vis spectroscopy for formulations F1-F4 at T0 to 12 weeks at 5±3° C., 25±2° C. and 60%±5% relative humidity (RH), or 40° C.±2° C. and 75%±5% relative humidity. FIG. 5 shows the clarity and opalescence of solutions (turbidity) for formulations F1-F4 at T0 to 12 weeks at 5±3° C., 25±2° C. and 60%±5% relative humidity (RH), or 40° C.±2° C. and 75%±5% relative humidity. FIGS. 6A-6D shows the count of subvisible particles for formulations F1-F4 at T0 to 12 weeks at 5±3° C., 25±2° C. and 60%±5% relative humidity (RH), or 40° C.±2° C. and 75%±5% relative humidity for particles ≥2 μm (FIG. 6A), particles ≥5 μm (FIG. 6B), particles ≥10 μm (FIG. 6C), and particles ≥25 μm (FIG. 6D). FIGS. 7A-7D provides the results of size exclusion HPLC (SE-HPLC) for formulations F1-F4 at T0 to 12 weeks at 5±3° C., 25±2° C. and 60%±5% relative humidity (RH), or 40° C.±2° C. and 75%±5% relative humidity by the % area of the main peak (FIG. 7A), the % area of the high molecular weight species (HMWS) (FIG. 7B), and the % area of the low molecular weight species (LMWS) (FIG. 7C). FIG. 7D provides an overlay of SE-HPLC chromatograms for formulations F1-F4 after 12 weeks at 40° C.±2° C. and 75%=5% relative humidity. FIGS. 8A-8D provides the results of imaged capillary isoelectric focusing (icIEF) for formulations F1-F4 at T0 to 12 weeks at 5±3° C., 25±2° C. and 60%±5% relative humidity (RH), or 40° C.±2° C. and 75%±5% relative humidity by the % area of the main peak (FIG. 8A), the % area of the acidic variants (FIG. 8B), and the % area of the basic variants (FIG. 8C). FIG. 8D provides an overlay of the icIEF data for formulations F1-F4 after 12 weeks at 40° C.±2° C. and 75%±5% relative humidity. FIGS. 9A-9D shows the results of reverse phase HPLC (RP-HPLC) for formulations F1-F4 at T0 to 12 weeks at 5±3° C., 25±2° C. and 60%±5% relative humidity (RH), or 40° C.±2° C. and 75%=5% relative humidity by the % area of the main peak under non-reducing conditions (FIG. 9A), by the % area of the LC of Antibody A under reducing conditions (FIG. 9B), by the % area of the HC of Antibody A under reducing conditions (FIG. 9C), and by the % area of the LC plus the % area of the HC of Antibody A under reducing conditions (FIG. 9D). FIGS. 10A-10E shows the results of capillary electrophoresis sodium dodecyl sulfate (CE-SDS) by Labchip for formulations F1-F4 at T0 to 12 weeks at 5±3° C., 25±2° C. and 60%±5% relative humidity (RH), or 40° C.±2° C. and 75%±5% relative humidity by % area of the main peak under non-reducing conditions (FIG. 10A), by the % area of the LC of Antibody A under reducing conditions (FIG. 10B), by the % area of the HC of Antibody A under reducing conditions (FIG. 10C). FIG. 10D provides overlays of CE-SDS data under reducing conditions for formulations F1-F4 after 12 weeks at 40° C.±2° C. and 75%±5% relative humidity. FIG. 10E provides overlays of CE-SDS data under non-reducing conditions for formulations F1-F4 after 12 weeks at 40° C.±2° C. and 75%±5% relative humidity. FIG. 11 shows the PS80 content in % (w/v) determined by a fluorescent micelle assay for formulations F1-F4 at T0 to 12 weeks at 5±3° C., 25±2° C. and 60%±5% relative humidity (RH), or 40° C.±2° C. and 75%±5% relative humidity. FIG. 12 shows the pH for formulations F1-F4 at T0 to 12 weeks at 5±3° C., 25±2° C. and 60%±5% relative humidity (RH), or 40° C.±2° C. and 75%±5% relative humidity.

Over the first twelve weeks of stability studies, no changes were seen for protein content, pH, or color for all tested conditions. Sub-visible particles results are shown in FIGS. 6A-6D. After 12 weeks at 5° C. and 25° C., no visible particles were observed in any formulation, while subvisible particle counts were lowest for the 150 mg/mL formulations at pH 6.2 (F2 and F3), followed by the 150 mg/mL formulation at pH 6.5 (F1) and the reference formulation at 60 mg/mL and pH 6.7 (F4). Overall low subvisible particle counts above 10 and 25 μm and below the USP <787> acceptance limits were measured at 5 and 25° C. Clarity and opalescence (turbidimetry) results are shown in FIG. 5. All tested formulations had an initial turbidity ≥20NTU but still below 30NTU, which corresponds to equally or less opalescent than reference suspension IV (Ph. Eur.). In general, lower turbidity levels were observed for F3 compared to F1 and F2. Turbidity values remained stable upon storage at 5° C. and 25° C. Turbidity increase measured from 4 weeks onward under all storage conditions was related to equipment.

Purity by SE-HPLC results are shown in FIGS. 7A-7D. No major changes were observed by SE-HPLC for all formulations at 5° C. and 25° C. Purity by CE-SDS results are shown in FIGS. 10A-10E. No significant changes in purity were observed by CE-SDS analysis after storage at 5° C. and 25° C.

At 40° C., all 150 mg/mL formulations (F1-F3) showed visible particles, haze, and subvisible particle counts from 2 weeks storage, while the reference formulation F4 remained free from visible particles at all time points. F1 also exhibited a slight increase in turbidity. At 40° C., changes observed for F1, F2, and F4 by SE-HPLC were comparable (FIGS. 7A-7D). Only at the 12 weeks timepoint, higher amount of LMWS were detected for F2 at 40° C. F3 showed lowest aggregate formation throughout storage. After 12 weeks, purity losses measured by CE-SDS at 40° C. were similar for all tested formulations except F4, which exhibited the smallest main peak under non-reduced conditions (FIGS. 10A-10D). Under reduced conditions, loss in heavy chain was likewise more pronounced for F4 (FIGS. 10A-10D).

RP-HPLC analysis under non-reduced conditions showed no changes upon storage for 12 weeks at 5° C. (FIGS. 9A-9D). Purity losses at 25° C. and 40° C. were comparable for all tested formulations. At 5° C., no major changes were observed for purity by icIEF analysis (FIGS. 8A-8D). However, pH dependent reduction of main peak and increase in acidic and basic variants were observed at 25° C. and 40° C. Higher pH formulations F1 (pH 6.5) and F4 (pH 6.7) had lower main peak levels and higher content of acidic species.

A rapid reduction in surfactant content was seen for all formulations and at all temperatures (FIG. 11). Based on the results of the 12-weeks stability study, formulation F3 was selected as the 150 mg/mL formulation for further study. Subsequently, only F3 and F4 formulations were further monitored at the 6-, 9-, and 12-month timepoints.

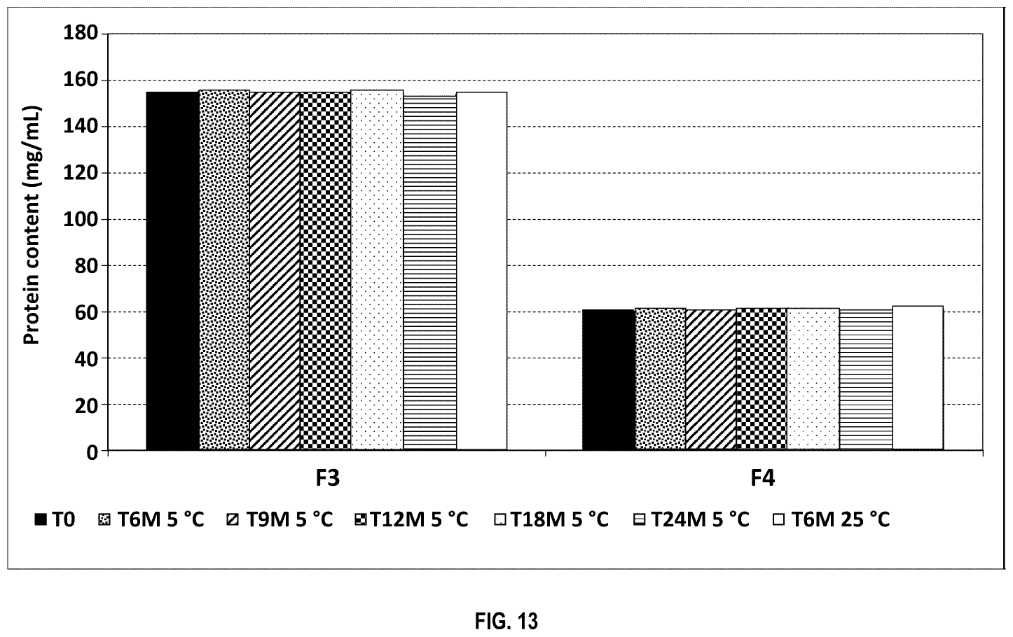

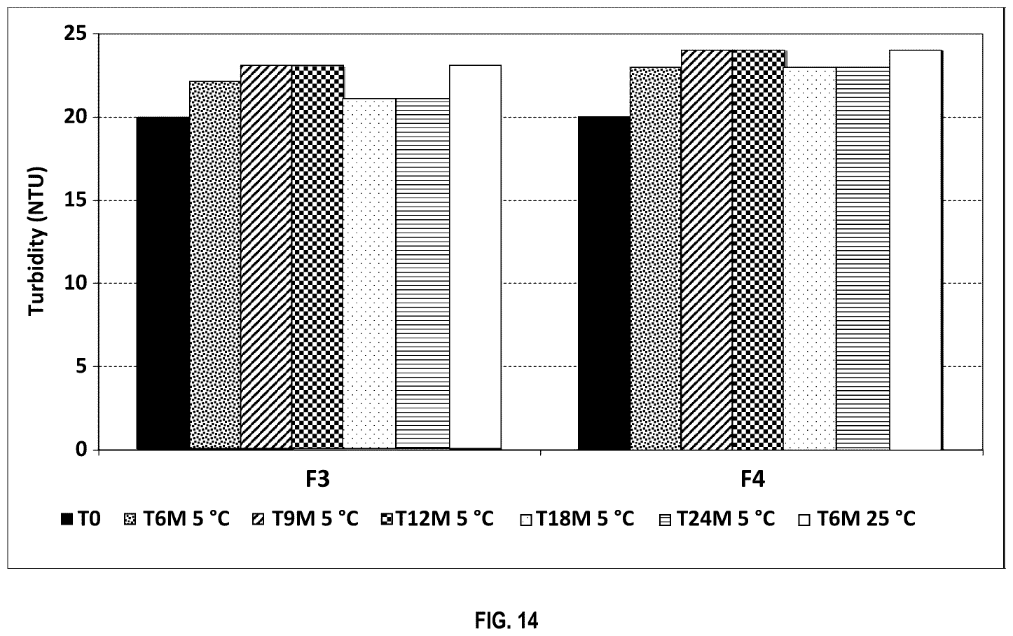

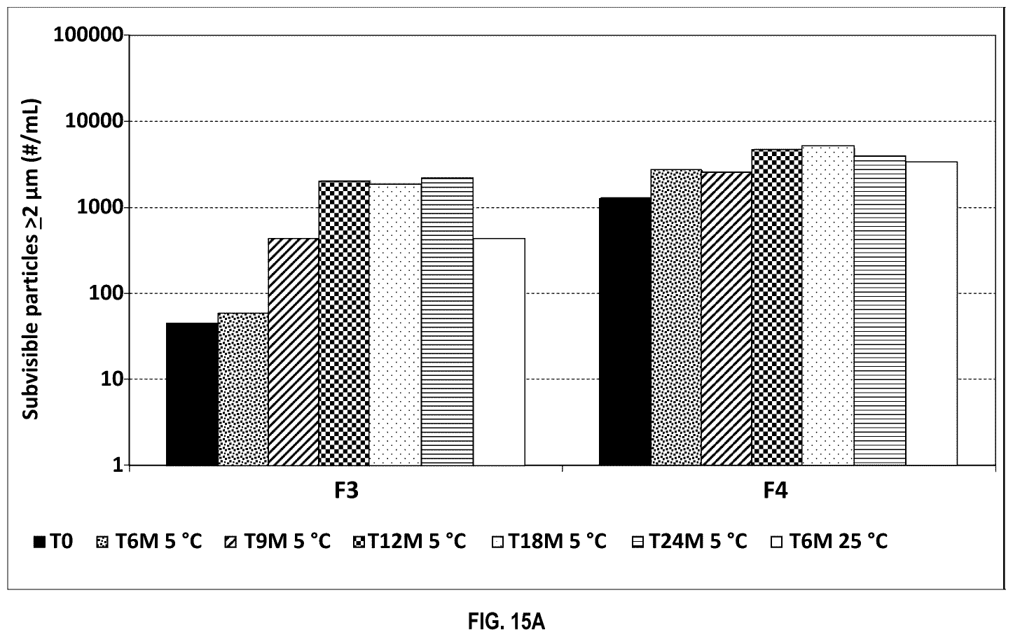

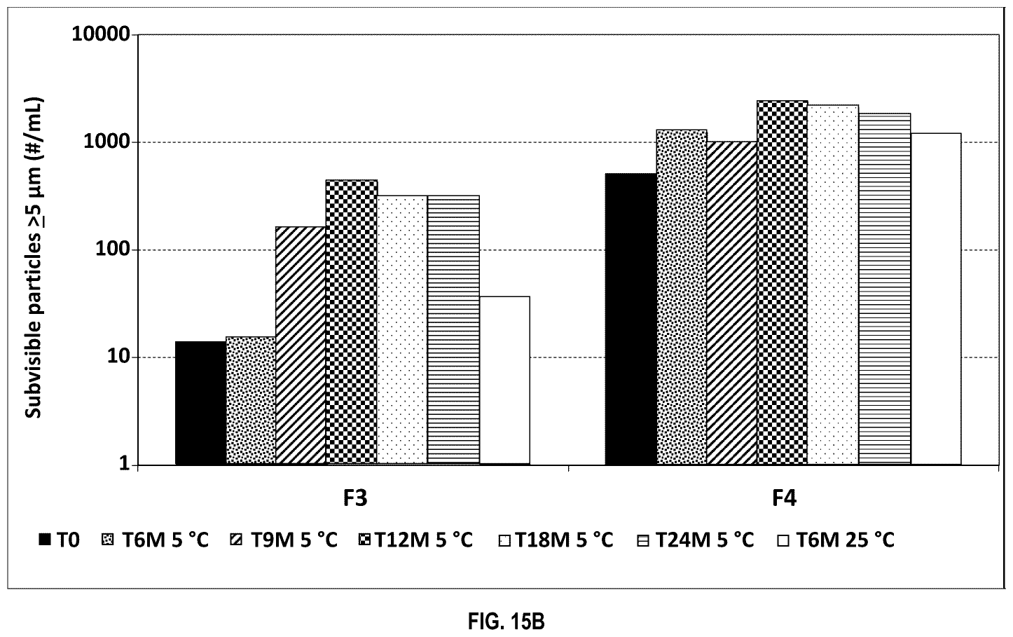

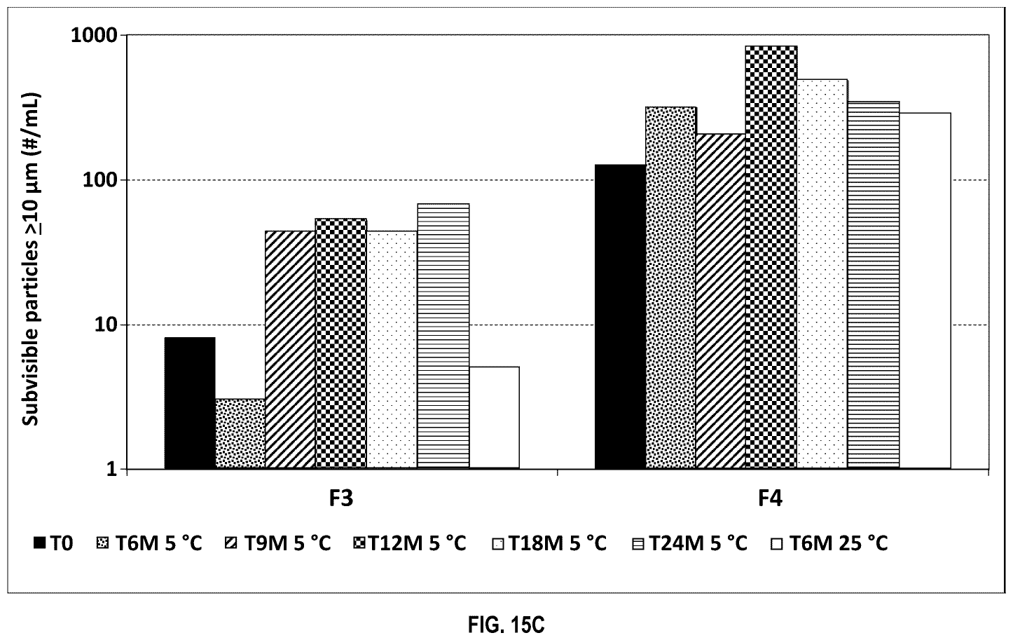

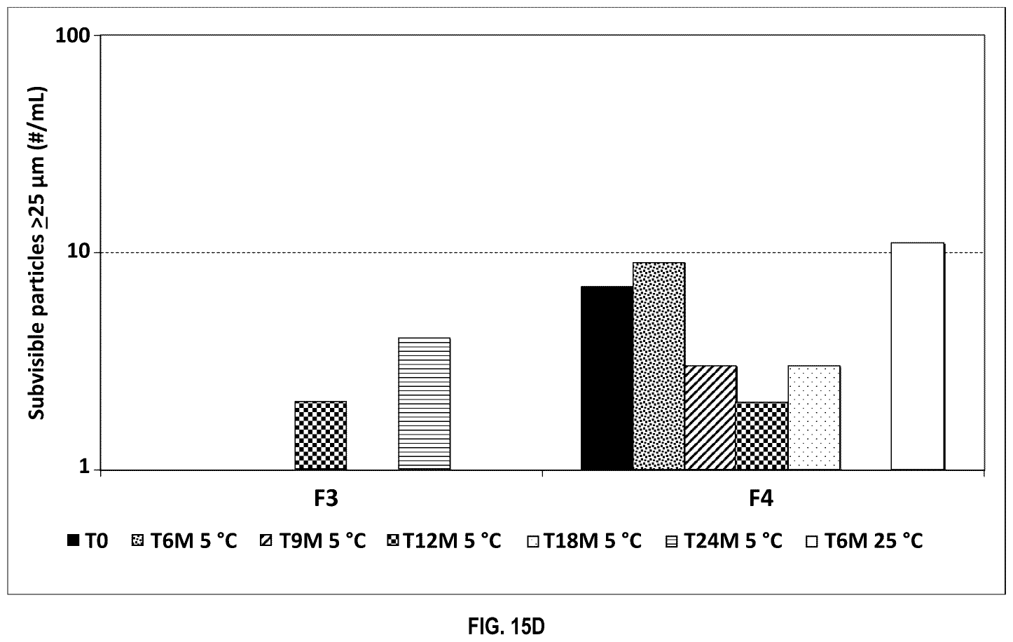

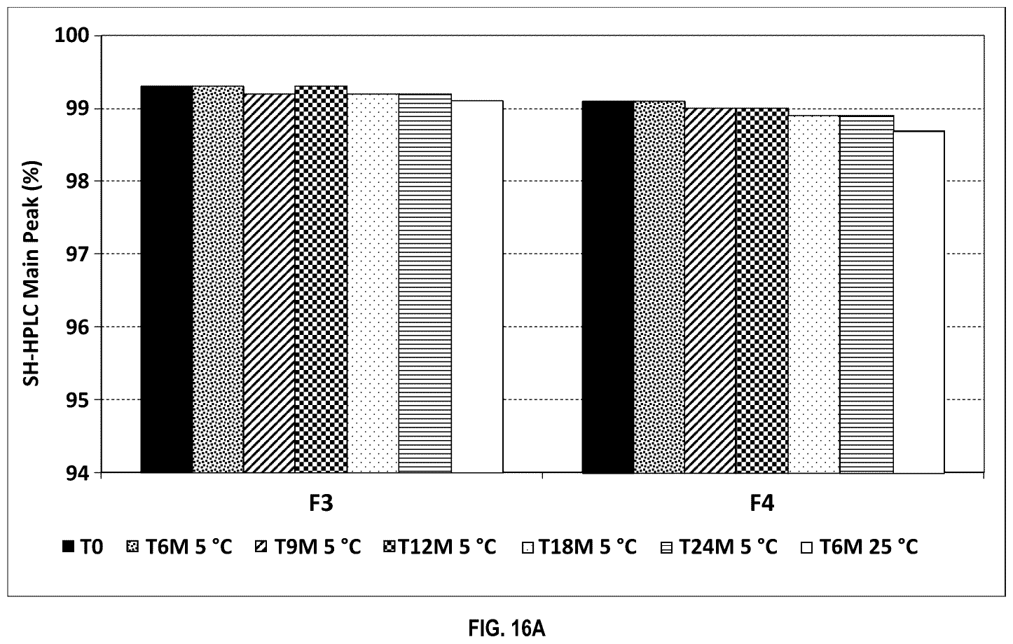

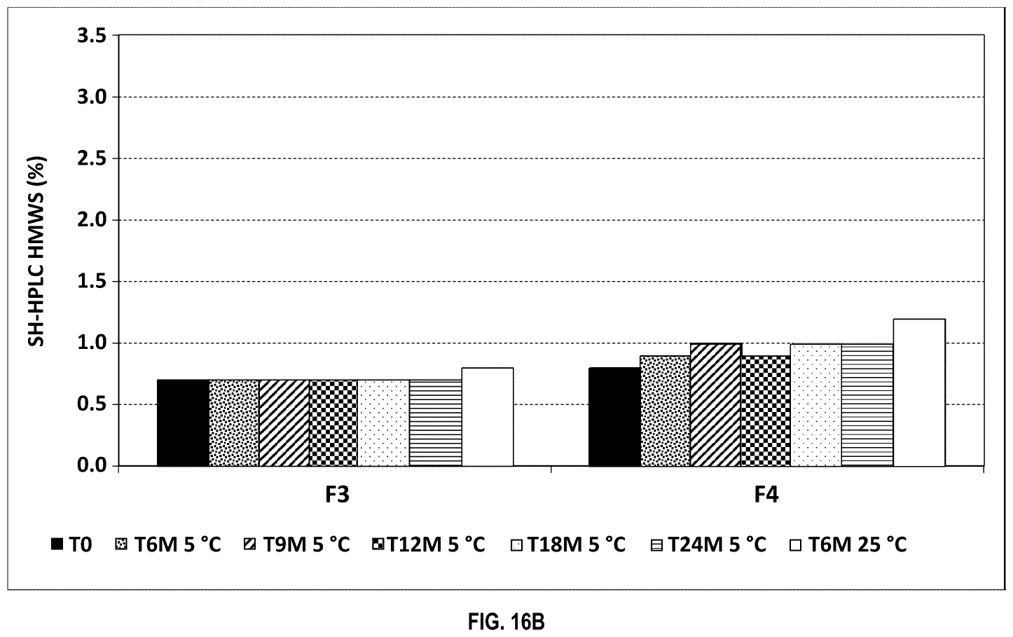

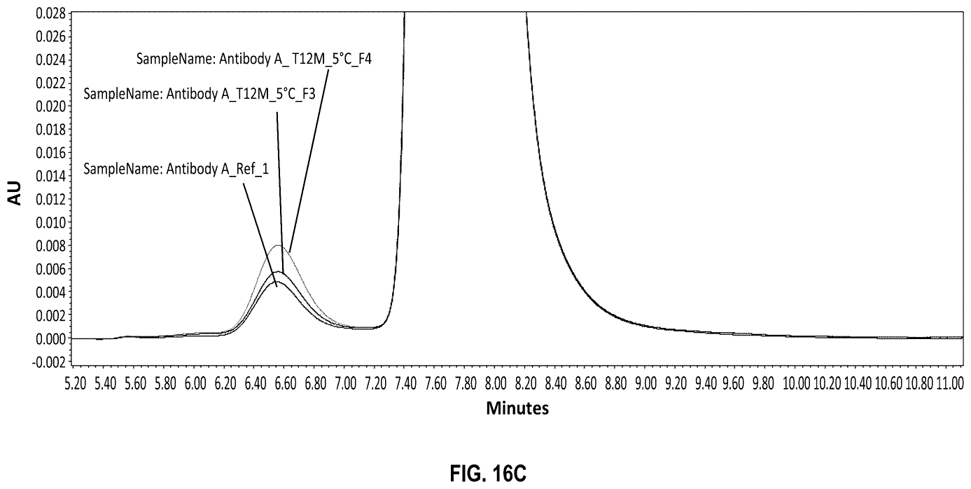

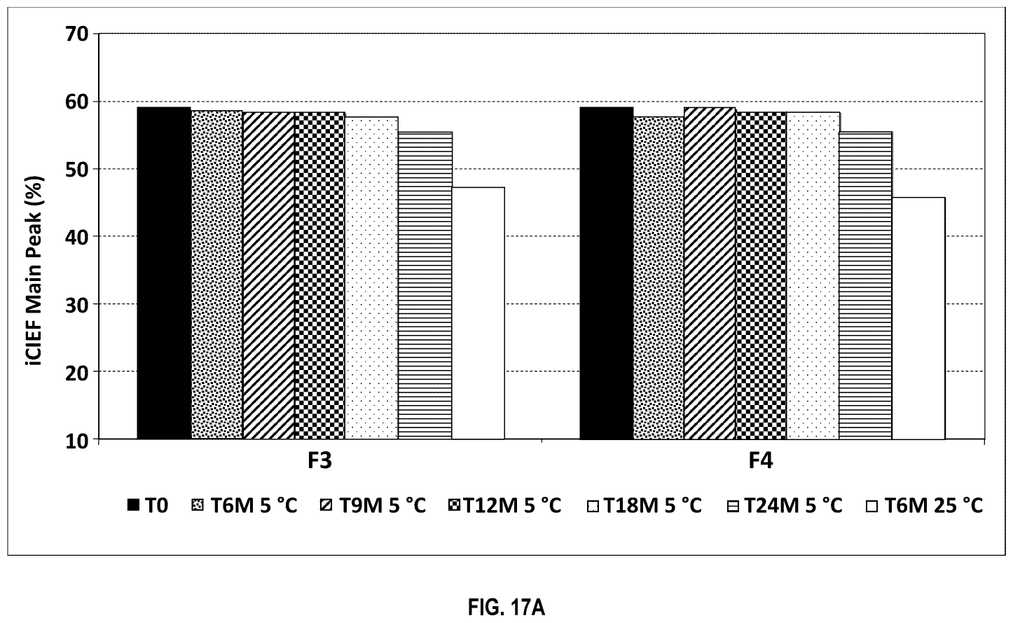

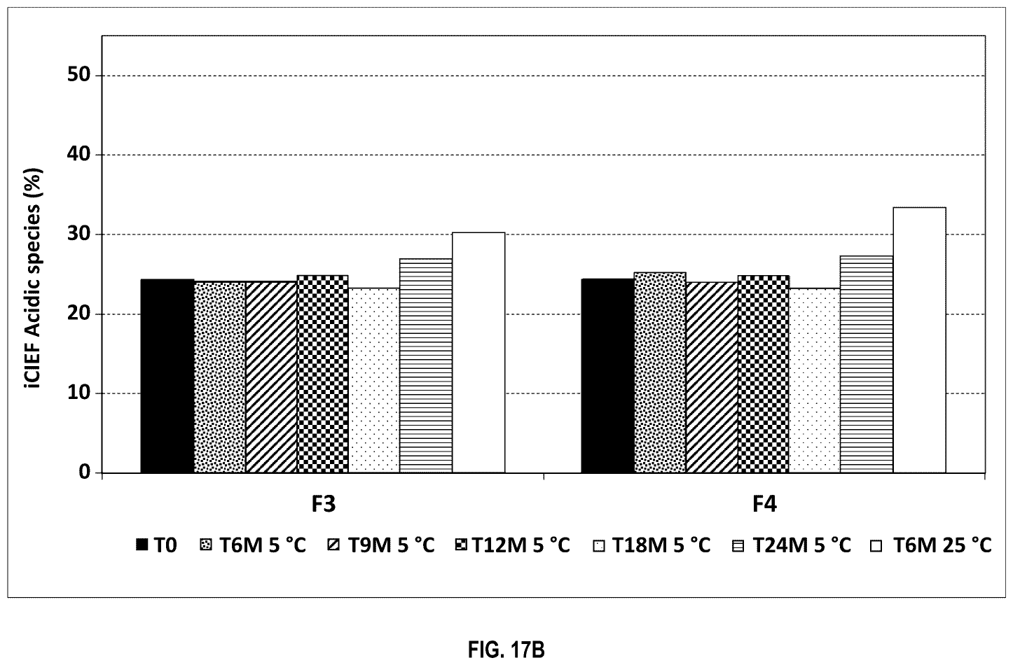

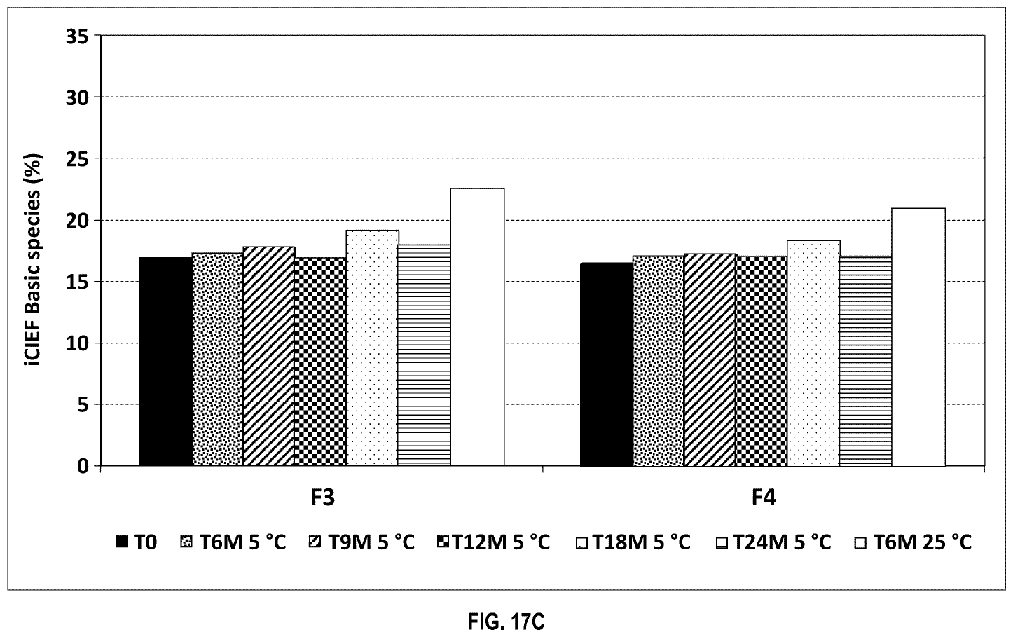

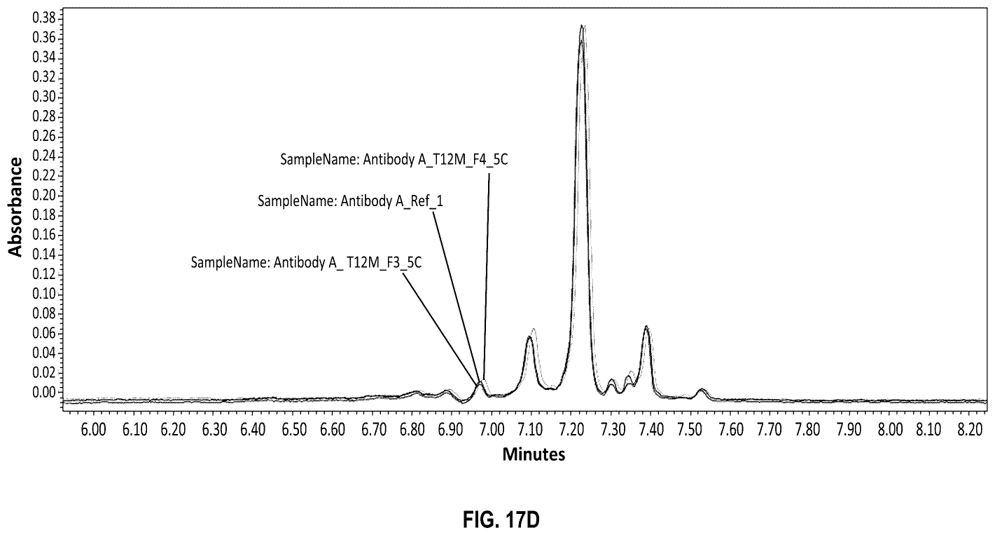

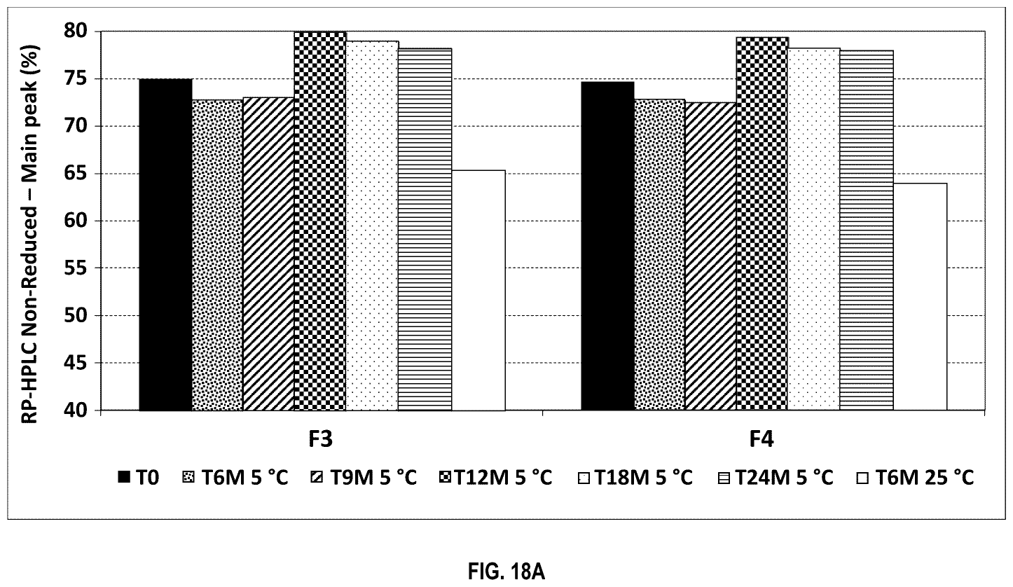

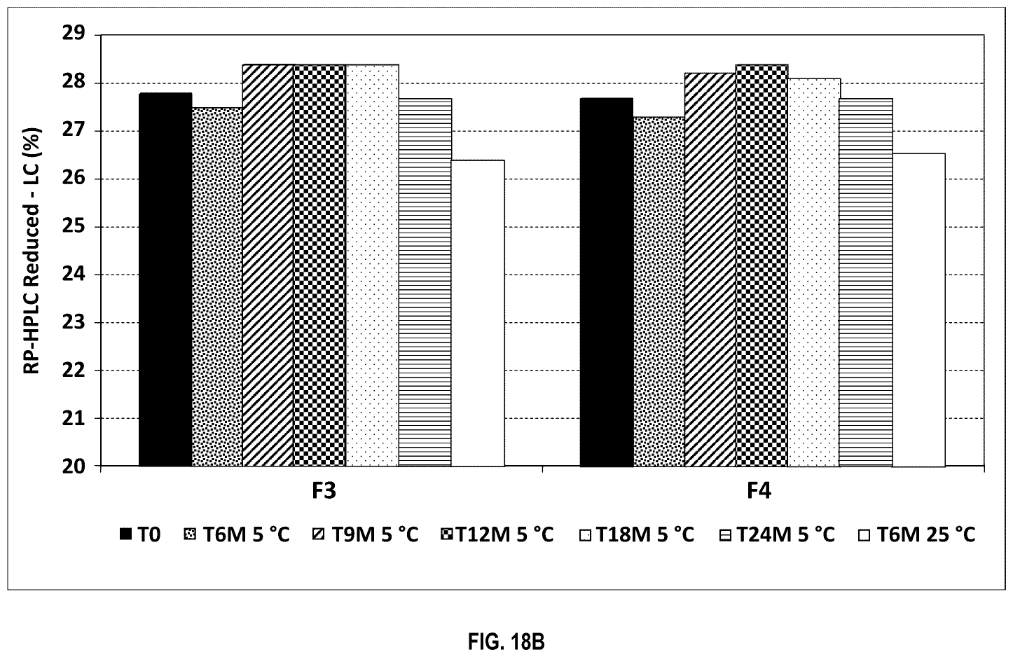

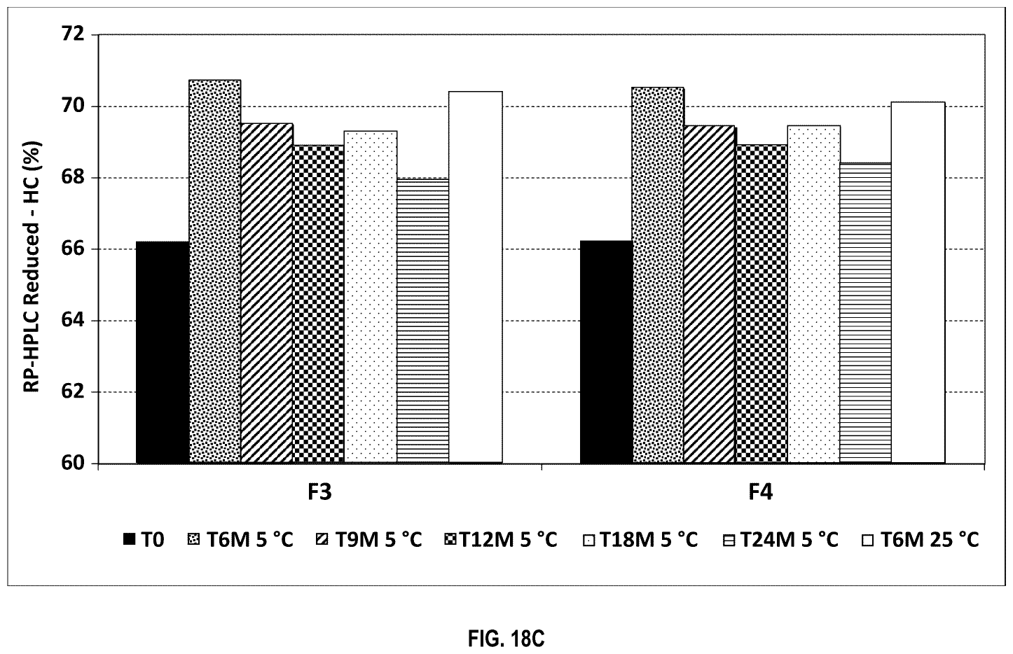

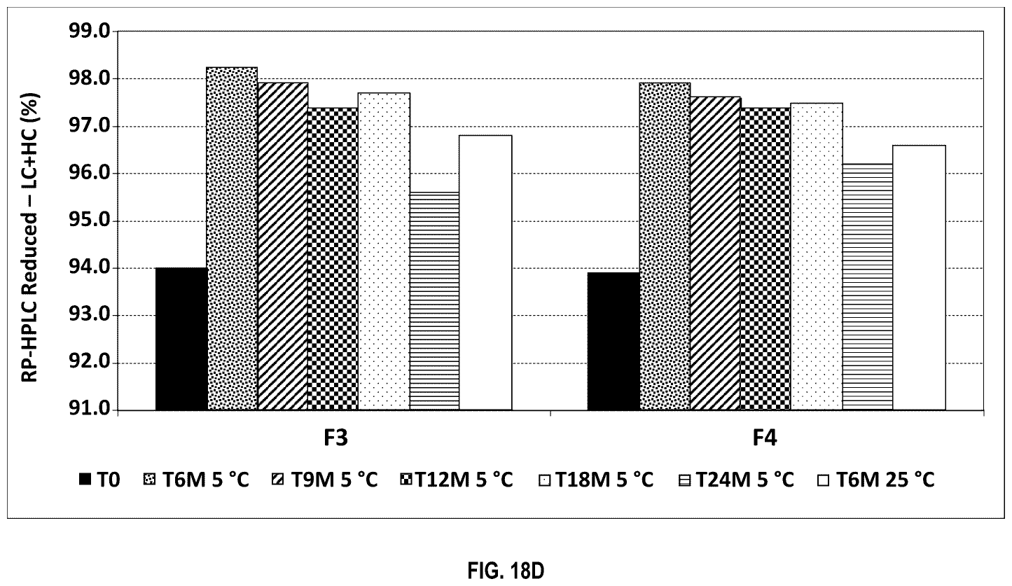

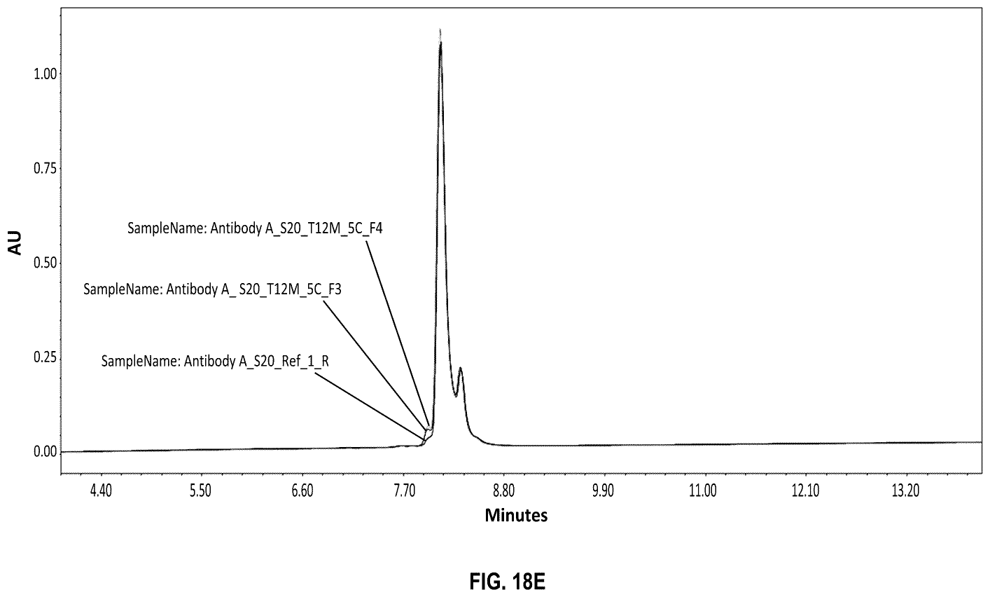

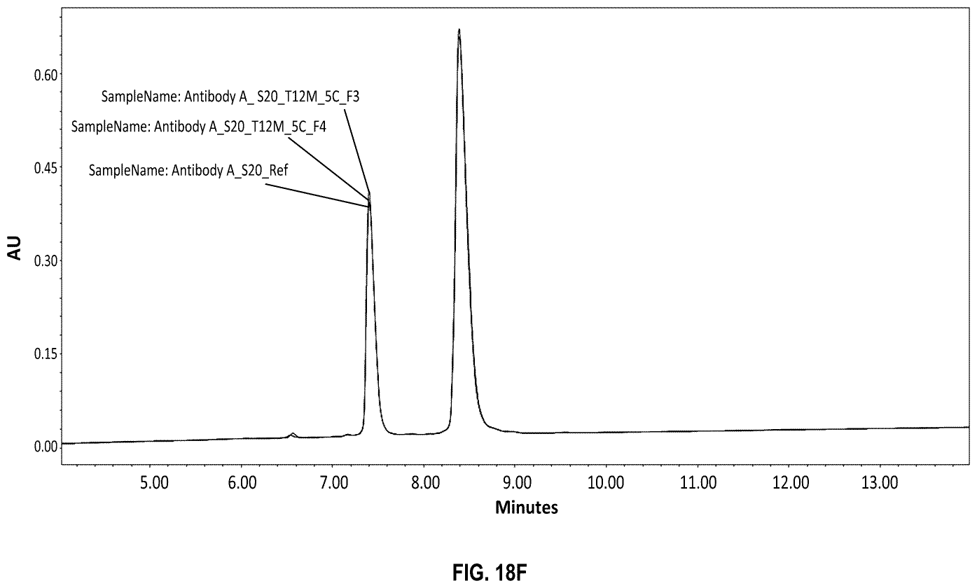

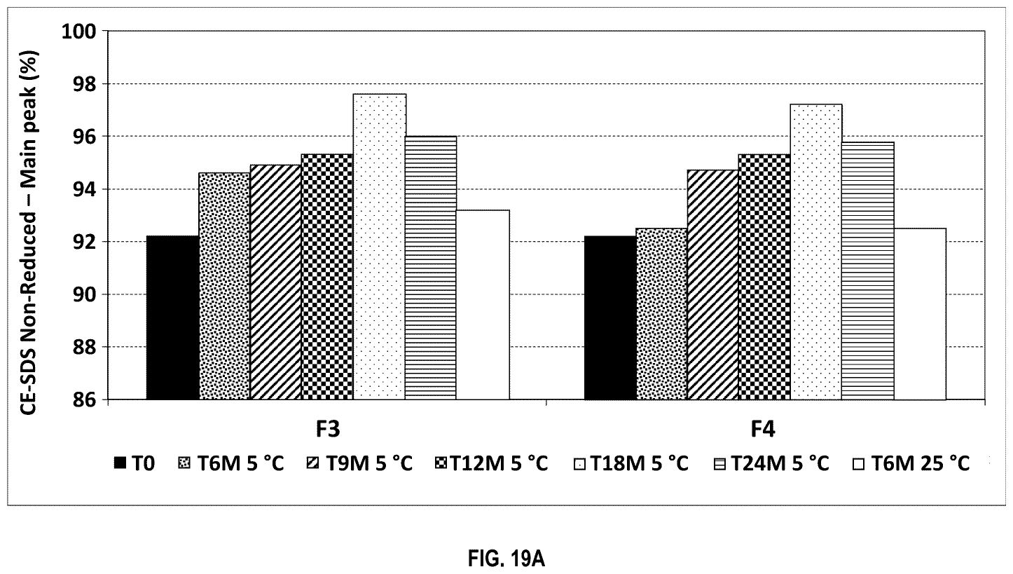

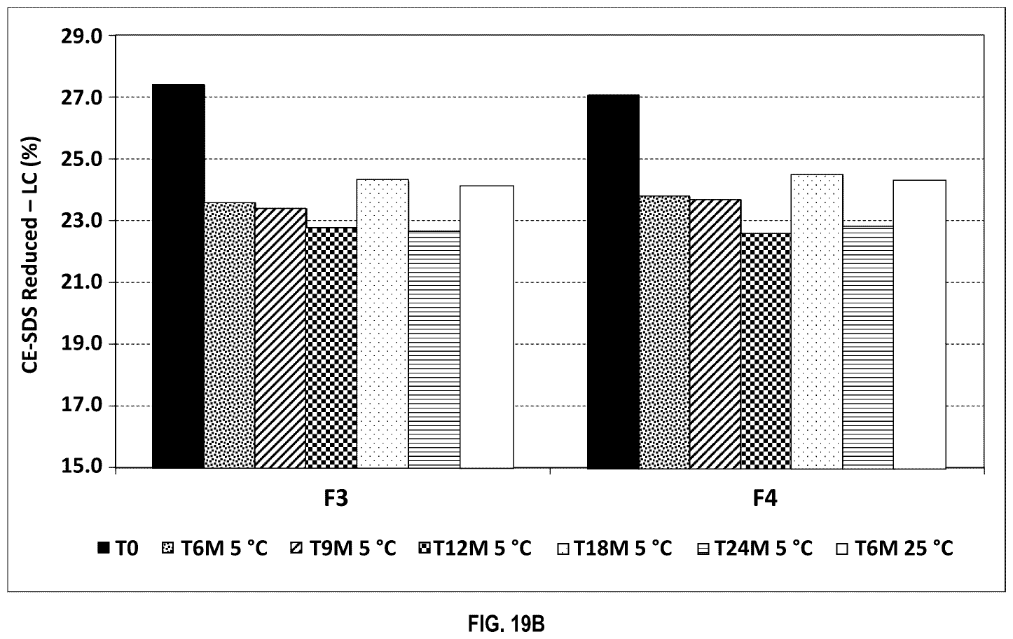

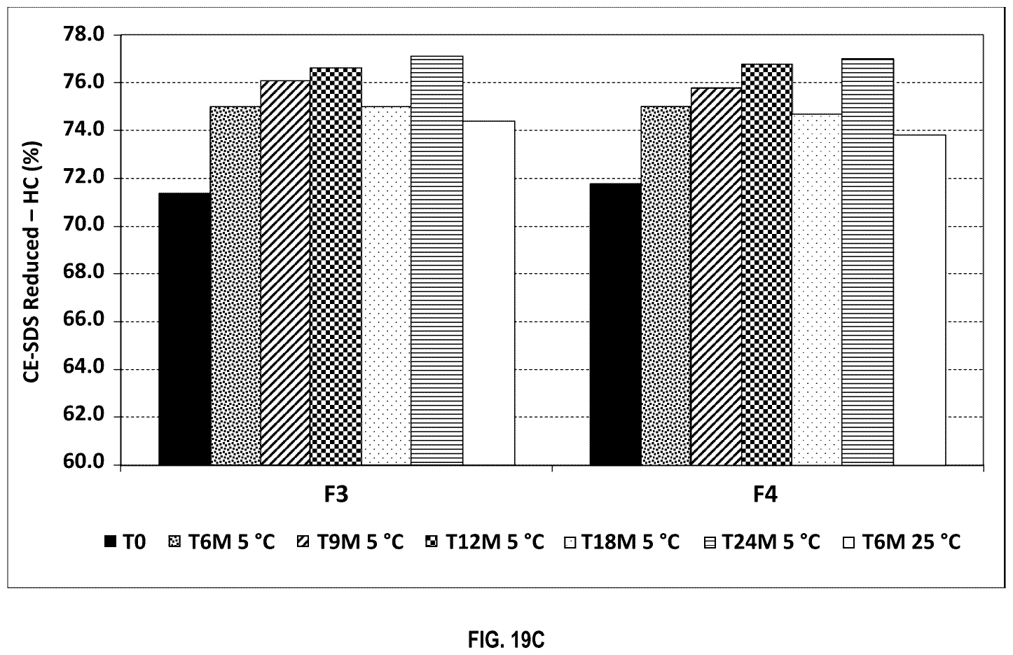

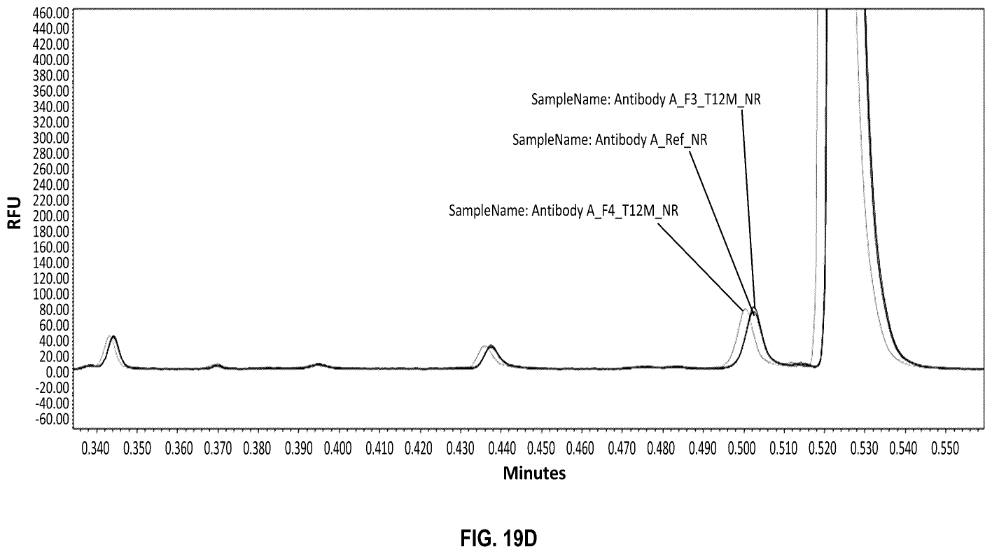

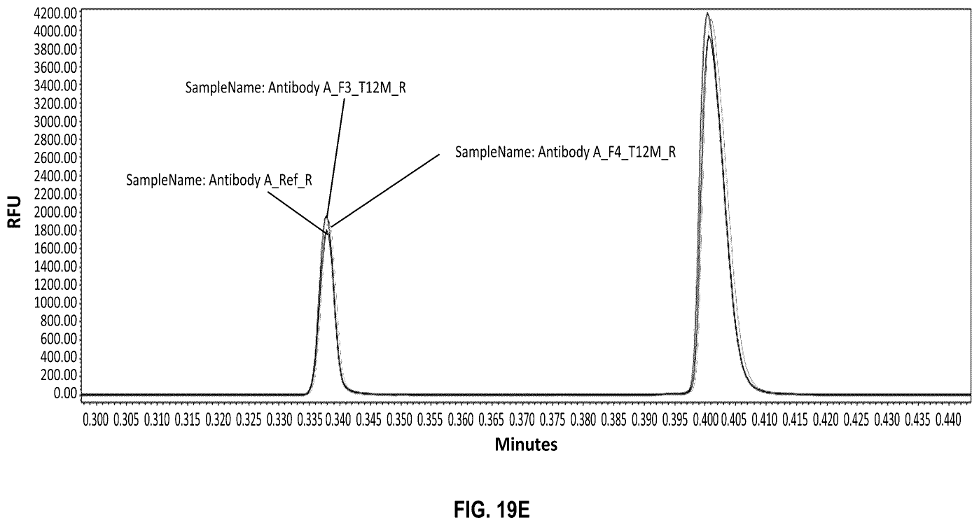

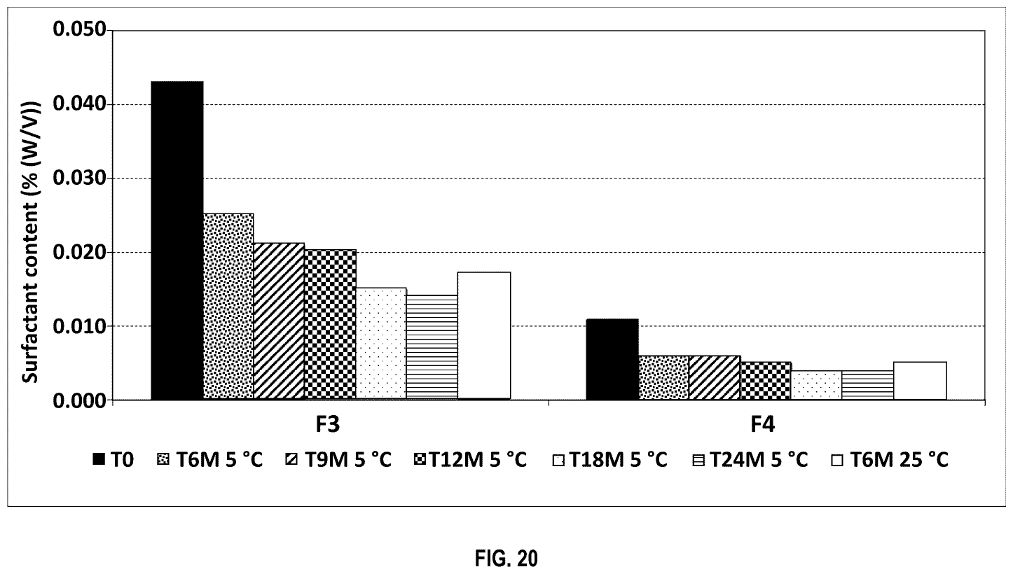

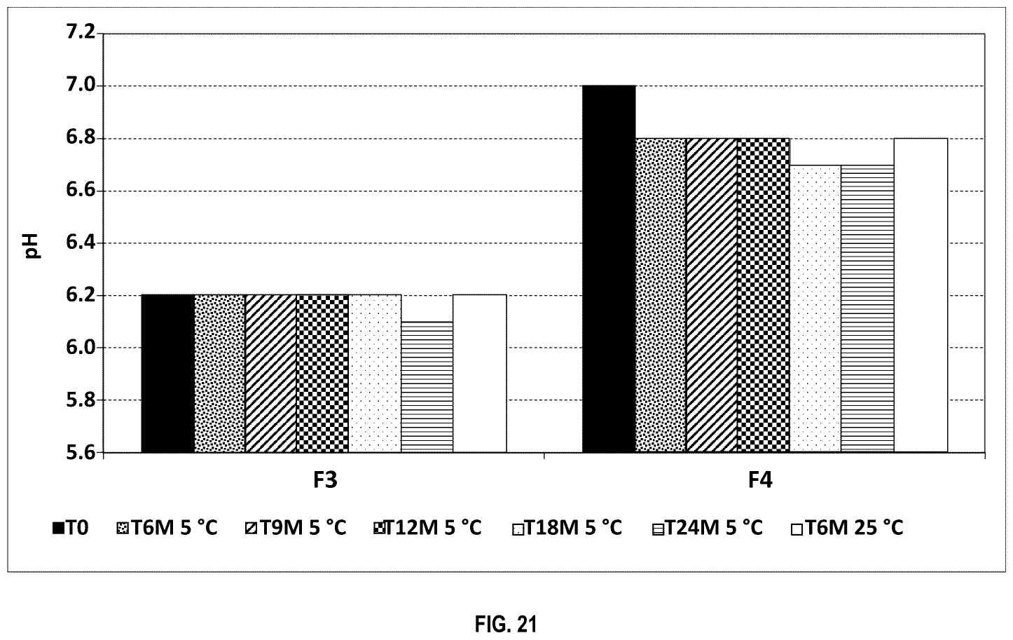

Long-term stability: T0 to 6-24 months. Results of long-term stability studies for F3 and F4 upon storage for up to 24 months are shown in FIGS. 13-21. FIG. 13 shows the total protein content (mg/mL) determined by UV/vis for formulations F3 and F4 at T0 at room temperature (25° C.); at T6M, T9M, T12M, T18M, and T24M at 5±3° C.; and at T6M at 25±2° C. and 60%±5% relative humidity (RH). FIG. 14 shows the clarity and opalescence of solution (turbidity in NTU) for formulations F3 and F4 at T0 at room temperature (25° C.); at T6M, T9M, T12M, T18M, and T24M at 5±3° C.; and at T6M at 25±2° C. and 60%±5% relative humidity (RH). FIGS. 15A-15D shows the subvisible particle count for formulations F3 and F4 at T0 at room temperature (25° C.); at T6M, T9M, T12M, T18M, and T24M at 5±3° C.; and at T6M at 25±2° C. and 60%±5% relative humidity (RH) for particles ≥2 μm (#/mL) (FIG. 15A), particles ≥5 μm (#/mL) (FIG. 15B), particles ≥10 μm (#/mL) (FIG. 15C), and particles ≥25 μm (#/mL) (FIG. 15D). FIGS. 16A-16C provides the purity results by size exclusion HPLC (SE-HPLC) for formulations F3 and F4 at T0 at room temperature (25° C.); at T6M, T9M, T12M, T18M, and T24M at 5±3° C.; and at T6M at 25±2° C. and 60%±5% relative humidity (RH) by the % area of the main peak (FIG. 16A) and the % area of the high molecular weight species (HMWS) (FIG. 16B). The low molecular weight species (LMWS) for F3 and F4 were either not detected or under the limit of detection at the times and under the conditions tested. FIG. 16C provides an overlay of SE-HPLC data for formulations F3 and F4 after 12 months at 5±3° C. FIGS. 17A-17D provides the results of imaged capillary isoelectric focusing (icIEF) for formulations F3 and F4 at T0 at room temperature (25° C.); at T6M, T9M, T12M, T18M, and T24M at 5±3° C.; and at T6M at 25±2° C. and 60%±5% relative humidity (RH) by the % area of the main peak (FIG. 17A), the % area of the acidic variants (FIG. 17B), and the % area of the basic variants (FIG. 17C). FIG. 17D provides an overlay of icIEF data for formulations F3 and F4 after 12 months at 5±3° C. FIGS. 18A-18F shows the results of reverse phase HPLC (RP-HPLC) for formulations F3 and F4 at T0 at room temperature (25° C.); at T6M, T9M, T12M, T18M, and T24M at 5±3° C.; and at T6M at 25±2° C. and 60%±5% relative humidity (RH) by the % area of the main peak under non-reducing conditions (FIG. 18A), by the % area of the LC of Antibody A under reducing conditions (FIG. 18B), by the % area of the HC of Antibody A under reducing conditions (FIG. 18C), and by the % area of the LC plus the % area of the HC of Antibody A under reducing conditions (FIG. 18D). FIG. 18E provides an overlay of RP-HPLC data under non-reducing conditions for formulations F3 and F4 after 12 months at 5±3° C. FIG. 18F provides an overlay of RP-HPLC data under reducing conditions for formulations F3 and F4 after 12 months at 5±3° C. FIGS. 19A-19E shows the results of capillary electrophoresis sodium dodecyl sulfate (CE-SDS) by Labchip for formulations F3 and F4 at T0 at room temperature (25° C.); at T6M, T9M, T12M, T18M, and T24M at 5±3° C.; and at T6M at 25±2° C. and 60%±5% relative humidity (RH) by % area of the main peak under non-reducing conditions (FIG. 19A), by the % area of the LC of Antibody A under reducing conditions (FIG. 19B), by the % area of the HC of Antibody A under reducing conditions (FIG. 19C). FIG. 19D provides an overlay of CE-SDS data under non-reducing conditions for formulations F3 and F4 after 12 months at 5±3° C. FIG. 19E provides an overlay of CE-SDS data under reducing conditions for formulations F3 and F4 after 12 months at 5±3° C. FIG. 20 shows the PS80 content in % (w/v) determined by a fluorescent micelle assay for formulations F3 and F4 at T0 at room temperature (25° C.); at T6M, T9M, T12M, T18M, and T24M at 5±3° C.; and at T6M at 25±2° C. and 60%±5% relative humidity (RH). FIG. 21 shows the pH for formulations F3 and F4 at T0 at room temperature (25° C.); at T6M, T9M, T12M, T18M, and T24M at 5±3° C.; and at T6M at 25±2° C. and 60%±5% relative humidity (RH).

6- to 12-Month Stability. After storage for up to 12 months at 5° C. and 6 months at 25° C., formulation F4 (60 mg/mL) exhibited many visible particles and haze formation. Formulation F3 (150 mg/mL) remained free of visible particles after storage for 9 months at 5° C. After 12 months storage, haze and visible particles were also observed for F3 at long term storage conditions. Color, content and pH of formulation F3 and F4 did not change for both temperatures. Turbidity values were stable and similar for both formulations. All values were below 30 NTUs after 12 months storage.

Subvisible particle levels were low for F3 upon storage for 9 months. Increase in particle levels were seen for 12 months timepoint at 5° C. for ≤2 μm≤10 μm particles. Higher levels of particles were detected for F4. In general, haze observation was correlating to increased sub-visible particle counts.

No major changes in purity were observed at 5° C. after 12 months storage. Formulation F3 and F4 showed comparable results in all tested methods. After 6 months storage at 25° C., formulation F4 showed a moderate increase in aggregates as assessed by SE-HPLC compared to F3. The icIEF profiles confirmed the purity changes observed after 3 months storage at 25° C. and 40° C. Reduction of main peak and increase in acidic and basic variants were observed in both formulations, formulation F4 showing a lower main peak level and higher content of acidic species compared to F3.

Finally, the polysorbate content decrease was further progressing at 25° C. and 5° C. to reach a content loss of 50% in both formulations upon 12 months storage at 5° C.

18-Month Stability (F3 and F4 only). At 5° C., many visible particles and haze were observed for both tested formulations after 18 months. Color, content, pH, and sub-visible particle levels did not change compared to the 12-month timepoint. After 18 months at 5° C., reference formulation F4 showed still higher sub-visible particle levels compared to formulation F3. No major changes in purity as assessed by SE-HPLC, CE-SDs, iCIEF and RP-HPLC were observed at 5° C. after 18 months storage. Both tested formulations showed comparable results in all tested methods. The polysorbate content continued to decrease in both F3 and F4 after storage for up to 18 months at 5° C., resulting in surfactant loss of more than 50%.

24-Month Stability (F3 and F4 only). Many visible particles were observed for both F3 and F4 after 24 months. Color, pH and sub-visible particle levels did not change compared to the 18-month timepoint. The protein content of formulation F3 decreased slightly compared to T18 months and compared to T0, however the result was still within method variability. After 24 months at 5° C., reference formulation F4 at low protein concentration of 60 mg/mL showed still higher sub-visible particle levels compared to high protein formulation F3. No major changes in purity as assessed by SE-HPLC, CE-SDs, iCIEF and RP-HPLC were observed at 5° C. after 24 months storage. Both tested formulations showed comparable results in all tested methods. Polysorbate content for F3 and F4 was similar compared to the 18-month timepoint.

### Example 6: Stability Testing of Formulation F3 of Antibody A (1 mL)

The stability of formulation F3 of Antibody A was evaluated under different storage conditions. Antibody A was formulated in 20 mM L-histidine/L-histidine HCl monohydrate, 50 mM L-arginine HCl, 110 mM sucrose, 10 mM methionine, 0.04% polysorbate 80 (w/v), pH 6.2 at concentration of 150 mg/mL and a nominal volume of 1.0 ml in a liquid state. The formulation was stored in 2R/13 MM clear Type 1 Fiolax® glass vials, SL, NBB, with 13 mm bromobutyl Flurotec® Serum Stoppers (FluroTec-13 mm Injection Stopper), and sealed with 13 mm aluminum TruEdge® flip-off seal (no text) Royal Blue.

The vials of Formulation 3 were stored at 5° C.±3° C. upright, 5° C.±3° C. inverted, 25° C./60% relative humidity upright, and 40° C./75% relative humidity upright at the timepoints indicated in Table 19.

TABLE 19

| | Time Point (months) | | | | | | | | | | |
|---|---|---|---|---|---|---|---|---|---|---|---|
| | BR/T0 | 1 M | 3 M | 6 M | 9 M | 12 M | 18 M | 24 M | 36 M | 42 M | 48 M |
| 5° C. ± 3° C. | X | X | X | X | X | X | X | X | X | — | — |
| (INV) | | | | | | | | | | | |
| 5° C. ± 3° C. | X | X | X | X | X | X | X | X | X | X | X |
| (UP) | | | | | | | | | | | |
| 25° C. ± 2° C./ | X | X | X | X | X | X | — | — | — | — | — |
| 60% RH ± | | | | | | | | | | | |
| 5% RH (UP) | | | | | | | | | | | |
| 40° C. ± 2° C./ | X | X | X | X | — | — | — | — | — | — | — |
| 75% RH ± | | | | | | | | | | | |
| 5% RH (UP) | | | | | | | | | | | |

Formulation F3 was evaluated for color, opalescence/clarity, pH at 24° C.±1° C., visible particles, subvisible particles ≥2 μm, subvisible particles ≥5 μm, subvisible particles ≥10 μm, subvisible particles ≥25 μm, protein content (determined by A280), surfactant content (HPLC), monomer content (SE-HPLC), aggregates (SE-HPLC), fragments (SE-HPLC), the sum of heavy chain plus light chain (reducing CE-SDS), total fragments (reducing CE-SDS), main peak (non-reducing CE-SDS), total other peaks (non-reducing CE-SDS), main isoform (icIEF), acidic isoforms (icIEF), basic isoforms (icIEF), and isoelectric point (pI) of main isoform (icIEF). The solution stored at 5° C.±3° C. upright was further tested for sterility and container closure integrity.

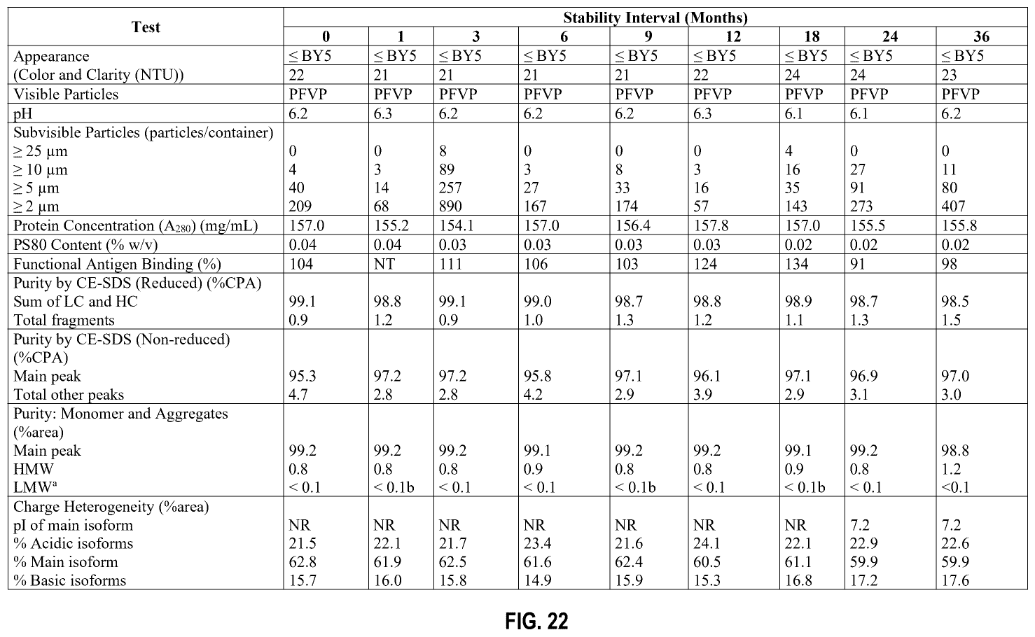

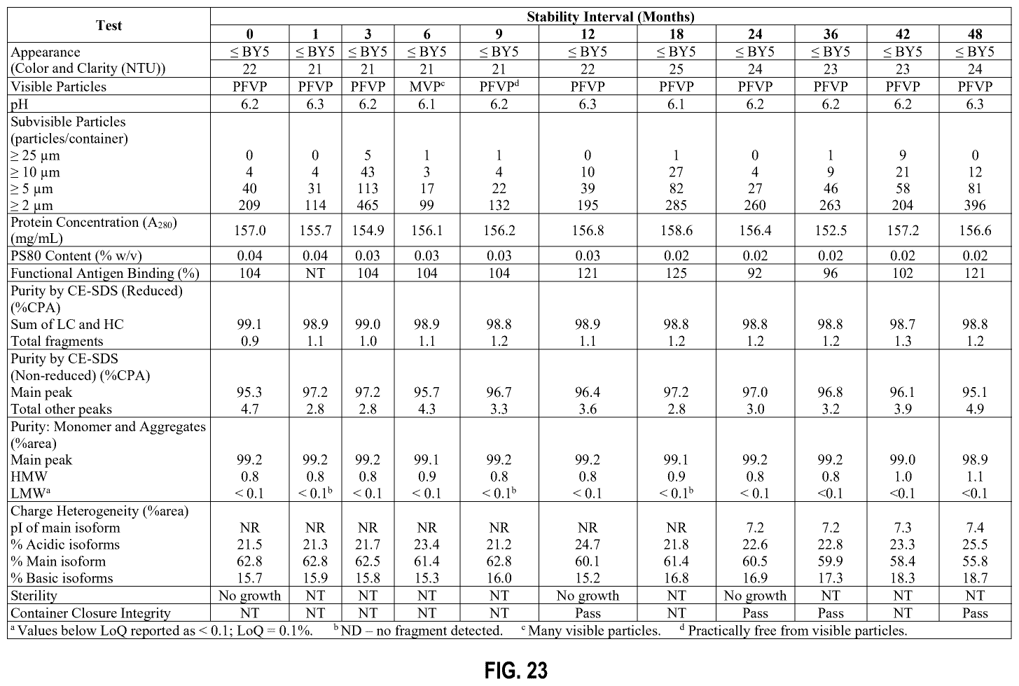

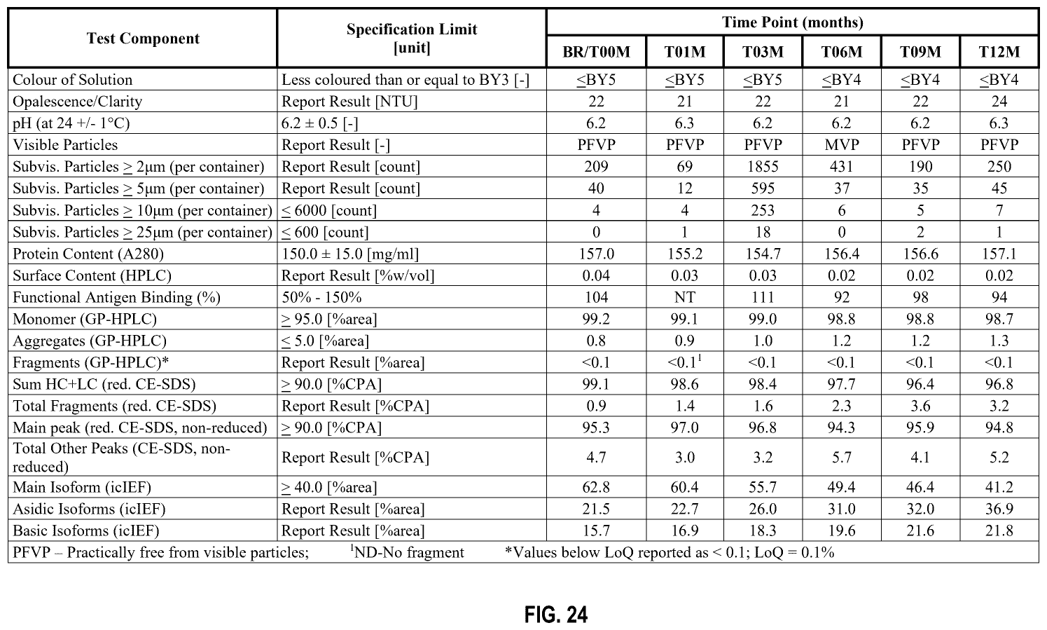

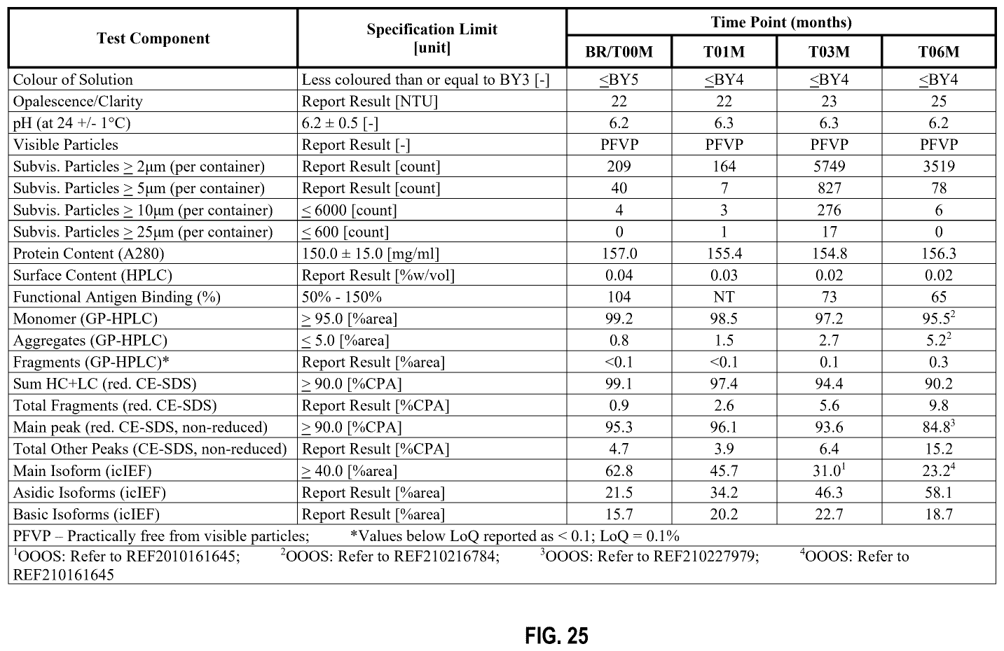

The results are summarized in FIGS. 22-25. FIG. 22 provides the results of stability testing of Formulation F3 of Antibody A at 5° C.±3° C. in inverted vials from T0 to 36 months. FIG. 23 provides the results of stability testing of Formulation F3 of Antibody A at 5° C.±3° C. in upright vials from T0 to 48 months. FIG. 24 provides the results of stability testing of Formulation F3 of Antibody A at 25° C./60% relative humidity from T0 to 12 months. FIG. 25 provides the results of stability testing of Formulation F3 of Antibody A at 40° C./75% relative humidity from T0 to 6 months.

After 36 months at storage conditions of at 5° C.±3° C., all results were within specifications, where defined. There was no difference observed between upright and inverted vial results. Testing of samples in upright vials at 5° C.±3° C. continued to 48 months, and all results remained within specifications, where defined. After 12 months at storage conditions of 25° C./60% relative humidity upright, a trend towards a decreased purity by icIEF, CE-SDS, and GP-HPLC was observed. The surfactant content results showed a decrease over time. At T06M, many visible particles were found in the sample. All quality attributes were within the specification limits, where defined. After 6 months at storage condition of 40° C./75% relative humidity upright, a more pronounced decrease of the main isoform (icIEF), with a concurrent increase in the acidic and basic isoforms (icIEF) was observed as compared to 25° C./60% relative humidity data. From T03M onwards, the results for main isoform (icIEF), were out of specification limit of ≥40.0% area. In addition, a continued decrease in purity by SE-HPLC and CE-SDS resulted in out-of-specification (OOS) at T06M for monomer (SE-HPLC), aggregates (SE-HPLC) and main peak (non-red. CE-SDS). The OOS observations were expected due to stressed sample storage condition. In addition, the surfactant content showed a decrease while opalescence/clarity appeared to increase over time. The rest of measured quality attributes were within the specification limits, where defined. Essentially no stability degradation was observed throughout the 48M while the formulation was frozen. The lack of trends up to 48M could support a shelf life of the frozen formulation for up to 60M.

### Example 7: Stability Testing of Bulk Formulation 3 of Antibody A (25 mL)

The stability of Antibody A was evaluated under different storage conditions in Formulation F3: 150 mg/mL Antibody A, 20 mM L-histidine/L-histidine HCl monohydrate, 50 mM L-arginine HCl, 110 mM sucrose, 10 mM methionine, 0.04% polysorbate 80 (w/v), having pH 6.2 and a nominal volume of 25 ml in 50 ml sterile PALL Allegro bags.

The bags of Formulation 3 were stored at −75° C.±10° C. (−65° C. or below) for thirty-six months, 5° C.±3° C. (+5° C.) for twelve months, or 25° C.±2° C./60%±5% relative humidity (+25° C./60% RH) for six months.

Formulation F3 was evaluated for clarity and degree of opalescence, degree of coloration, protein concentration (A280) (E^{0.1%}_{1 cm}=1.46), pH at 24° C.±1° C., isoelectric point (pI) of main isoform (icIEF), % main isoform (icIEF), % acidic isoforms (icIEF), % basic isoforms (icIEF), ≥90% IgG as heavy and light chains (Reduced SDS-PAGE), % total fragments (Reduced SDS-PAGE), % main peak and % total other peaks (Non-reduced SDS-PAGE), ≥95% main peak (SE-HPLC), ≤5% aggregates (SE-HPLC), and % fragments (SE-HPLC). The sample at +5° C. was further tested for bioburden.

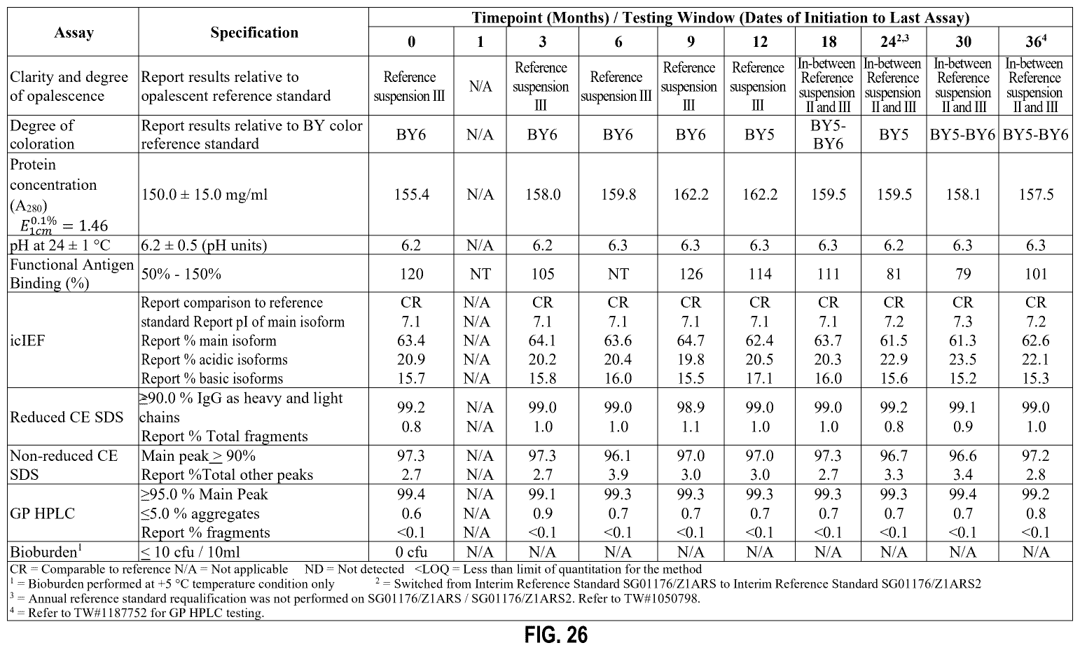

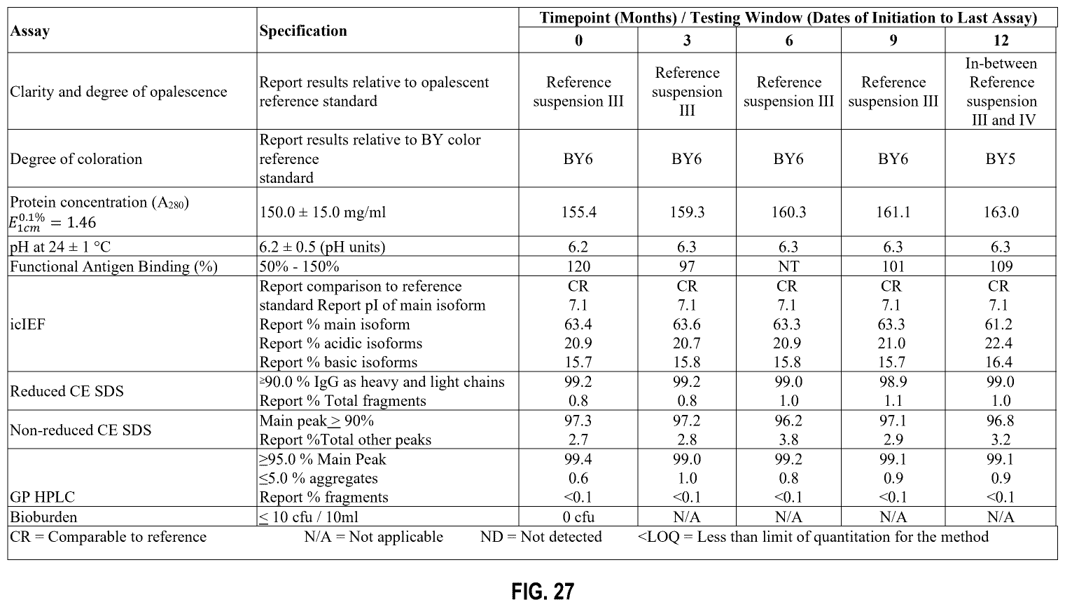

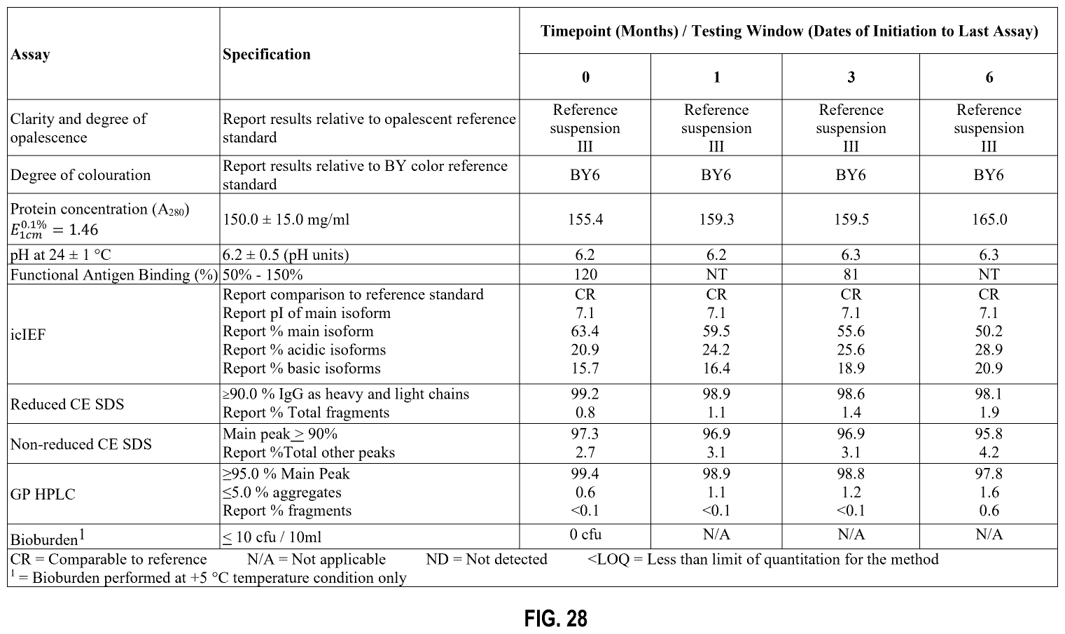

The results are summarized in FIGS. 26-28. FIG. 26 provides the results of stability testing of Formulation F3 of Antibody A at −75° C.±10° C. (−65° C. or below) from T0 to 36 months. FIG. 27 provides the results of stability testing of Formulation F3 of Antibody A at 5° C.±3° C. (+5° C.) from T0 to 12 months. FIG. 28 provides the results of accelerated stability testing of Formulation F3 of Antibody A at 25° C.±2° C./60%±5% relative humidity (+25° C./60% RH) from T0 to 6 months.

The samples stored at −65° C. or below for 36 months passed the stability study specification by all test methods. Changes were observed in icIEF analysis compared to study start. In the icIEF analysis for the sample stored at −65° C. or below, the percentage in acidic isoform increased from 20.9% at T=0 to 22.1% at 36 months. Antibody A in Formulation 3 was confirmed to be acceptably stable for 36 months when stored at −65° C. or below. A shelf life of thirty-six months at −65° C. or below for Antibody A in Formulation 3 was confirmed based on the available data.

The samples stored at +5° C. for twelve months passed the stability study specification by all test methods. Changes were observed in the Protein Concentration analysis at twelve months compared to T0, with the protein concentration increasing from 155.4 mg/mL at T0 to 163.0 mg/mL at 12 months. However, there were no significant changes observed for the sample stored at +5° C. in the other test methods compared to the study start. Changes in the protein concentration did not seem to impact the performance of the sample stored at this temperature condition at 12 months as indicated by the other test methods.

The samples stored at +25° C./60% RH for six months passed the stability study specification by all test methods. Changes were observed in icIEF, CE SDS, SE HPLC (GP HPLC), and Protein Concentration analysis at six months compared to T0.

In the icIEF analysis for the sample stored at +25° C./60% RH, there was a decrease in percentage of main isoform from 63.4% at T=0 to 50.2% at 6 months, an increase in percentage of acidic isoform from 20.9% at T=0 to 28.9% at 6 months, and an increase in percentage of basic isoform from 15.7% at T=0 to 20.9% at 6 months. In the CE SDS reduced and non-reduced analysis for the sample stored at +25° C./60% RH, there was a decrease in percentage IgG (heavy chains and light chains) from 99.2% at T=0 to 98.1% and there was a decrease in percentage intact IgG from 97.3% at T=0 to 95.8% at 6 months. In the SE HPLC (GP HPLC) analysis for the sample stored at +25° C./60% RH, there was a decrease in percentage of the main peak from 99.4% at T=0 to 97.8% at 6 months. In the Protein Concentration analysis for the sample stored at +25° C./60% RH, there was an increase in protein concentration from 155.4 mg/ml at T=0 to 165.0 mg/ml at 6 months.

These are signs of sample degradation which is expected for samples stored at the stressed storage condition.

### Example 8: Target Concentrations for Manufacturing Antibody A

Table 21 shows the target concentration of each excipient in a formulation of Antibody A along with the manufacturing ranges in bold. For example, the L-Arginine ranges are 49.75 mM to 50.22 mM with a target of 49.99 mM. The titer controls during manufacturing limit the pH of the formulation of Antibody A to a range of from 6.1-6.3.

TABLE 21

| | | | | Ranges (mM) | | |
|---|---|---|---|---|---|---|
| Component | Quantities (g) | | | Low | Target | High |
| Buffering agent: | 1791 | 1800 | 1809 | 11.54 | 11.60 | 11.66 |
| L-Histidine, Multi-Compendial | | | | | | |
| L-Histidine | 1752 | 1760 | 1768 | 8.36 | 8.40 | 8.43 |
| Monohydrochloride | | | | 19.90 | 20.00 | 20.09 |
| Colloidal stabilizer: | 10480 | 10530 | 10580 | 49.75 | 49.99 | 50.22 |
| L-Arginine, Hydrochloride | | | | | | |
| Multi-Compendial | | | | | | |
| Antioxidant: | 1483 | 1490 | 1497 | 9.94 | 9.99 | 10.03 |
| L-Methionine, Multi-Compendial | | | | | | |
| Saccharide: | 37470 | 37650 | 37830 | 109.47 | 109.99 | 110.52 |
| Sucrose, Multi-Compendial | | | | | | |
| Surfactant: | 99.5 | 100 | 100.5 | 9.95% | 10.00% | 10.05% |
| Polysorbate 80 | | | | | | |

### Example 9: Osmolality of the Formulation Mimicking Physiological Conditions Due to Additional of Saccharide

The saccharide in the formulation, sucrose, is also acting as both a conformational stabilizer and a tonicity agent, because the level of added saccharide (110 mM sucrose) raises the tonicity of the formulation, resulting in an osmolality of the formulation to physiological conditions. The osmolality of the formulation ranges from 243-343 mOsmo/Kg, achieving physiological conditions similar to blood (˜300 mOsmo/Kg). Injecting a product into a subject that has an osmolality closer to blood helps alleviate pain and discomfort. Other tonicity agents, such as trehalose, could be added to the formulation to achieve the same benefit as sucrose of achieving an osmolality of the formulation closer to that of blood.

### Example 10: Numbered Embodiments

The invention is further exemplified by the non-limiting numbered embodiments below.

Embodiment A1. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a heavy chain CDR1 having an amino acid sequence of SEQ ID NO: 1; a heavy chain CDR2 having an amino acid sequence of SEQ ID NO: 2; a heavy chain CDR3 having an amino acid sequence of SEQ ID NO: 3; a light chain CDR1 having an amino acid sequence of SEQ ID NO: 4; a light chain CDR2 having an amino acid sequence of SEQ ID NO: 5; and a light chain CDR3 having an amino acid sequence of SEQ ID NO: 6.

Embodiment A2. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a variable heavy chain region (VH) having an amino acid sequence of SEQ ID NO: 7.

Embodiment A3. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a variable light chain region (VL) having an amino acid sequence of SEQ ID NO: 8.

Embodiment A4. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a variable heavy chain region (VH) having an amino acid sequence of SEQ ID NO: 7 and a variable light chain region (VL) having an amino acid sequence of SEQ ID NO: 8.

Embodiment A5. A formulation comprising 100 mg/mL to 200 mg/mL anti-IL-1β antibody, a buffering agent, a colloidal stabilizer, an antioxidant, a saccharide, and a surfactant, wherein the anti-IL-1β antibody comprises a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 9 or SEQ ID NO: 10.

Embodiment A6. A formulation comprising 100 mg/mL to 200 mg/mL anti-IL-1β antibody, a buffering agent, a colloidal stabilizer, an antioxidant, a saccharide, and a surfactant, wherein the anti-IL-1β antibody comprises a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 11.

Embodiment A7. A formulation comprising 100 mg/mL to 200 mg/mL anti-IL-1β antibody, a buffering agent, a colloidal stabilizer, an antioxidant, a saccharide, and a surfactant, wherein the anti-IL-1β antibody comprises a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 9 or SEQ ID NO: 10 and a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 11.

Embodiment A8. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a heavy chain having an amino acid sequence of SEQ ID NO: 9 SEQ ID NO: 10.

Embodiment A9. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a light chain having an amino acid sequence of SEQ ID NO: 11.

Embodiment A10. A formulation comprising 100 mg/mL to 200 mg/mL anti-IL-1β antibody a buffering agent, a colloidal stabilizer, an antioxidant, a saccharide, and a surfactant, wherein the anti-IL-1β antibody comprises a heavy chain having an amino acid sequence of SEQ ID NO: 9 SEQ ID NO: 10 and a light chain having an amino acid sequence of SEQ ID NO: 11.

Embodiment A11. The formulation of any one of embodiments A1-A10, wherein the formulation comprises 10 mM to 30 mM buffering agent, 30 mM to 70 mM colloidal stabilizer, 1 to 20 mM antioxidant, 70 to 150 mM saccharide, and 0.01% to 0.04% (w/v) surfactant.

Embodiment A12. The formulation of any one of embodiments A1-A10, 150 mg/mL of the anti-IL-1β antibody, 20 mM buffering agent, 50 mM colloidal stabilizer, 10 mM antioxidant, 110 mM saccharide, and 0.02% to 0.04% (w/v) surfactant.

Embodiment A13. The formulation of any one of embodiments A1-A12, wherein the buffering agent is histidine/histidine HCl and/or sodium phosphate.

Embodiment A14. The formulation of any one of embodiments A1-A12, wherein the colloidal stabilizer is arginine/arginine HCl, lysine/lysine HCl, and/or sodium chloride.

Embodiment A15. The formulation of any one of embodiments A1-A12, wherein the antioxidant is methionine.

Embodiment A16. The formulation of any one of embodiments A1-A12, wherein the saccharide is sucrose, glucose, fructose, galactose, mannose, arabinose, xylose, erythrose, lactose, maltose, and/or trehalose.

Embodiment A17. The formulation of any one of embodiments A1-A12, wherein the surfactant is PS80.

Embodiment A18. The formulation of any one of embodiments A1-A12, wherein the buffering agent is histidine/histidine HCl, the colloidal stabilizer is arginine/arginine HCl, the antioxidant is methionine, the saccharide is sucrose, and the surfactant is PS80.

Embodiment A19. The formulation of any one of embodiments A1-A18, wherein the pH of the formulation is 6 to 7.

Embodiment A20. The formulation of any one of claims A1-A18, wherein the pH of the formulation is 6 to 6.4.

Embodiment A21. The formulation of any one of embodiments A1-A18, wherein the pH of the formulation is 6.2.

Embodiment A22. The formulation of any one of embodiments A1-A21, wherein the anti-IL-1β antibody comprises ≥95% monomeric antibody and ≤5% aggregated antibody. Embodiment A23. The formulation of any one of embodiments A1-A21, wherein the anti-IL-1β antibody comprises ≥97% monomeric antibody and ≤3% aggregated antibody. Embodiment A24. The formulation of any one of embodiments A1-A21, wherein the anti-IL-1β antibody comprises ≥98% monomeric antibody and ≤2% aggregated antibody. Embodiment A25. The formulation of any one of embodiments A1-A21, wherein the anti-IL-1β antibody comprises ≥99% monomeric antibody and ≤1% aggregated antibody.

Embodiment A26. The formulation of any one of embodiments A1-A21, wherein the anti-IL-1β antibody comprises ≤0.2% fragments.

Embodiment A27. The formulation of any one of embodiments A1-A21, wherein the anti-IL-1β antibody comprises ≤0.1% fragments.

Embodiment A28. The formulation of any one of embodiments A1-A21, where in the anti-IL-1β antibody comprises ≥50% of the main isoform as determined by imaged capillary isoelectric focusing (icIEF).

Embodiment A29. The formulation of any one of embodiments A1-A21, where in the anti-IL-1β antibody comprises ≥60% of the main isoform as determined by imaged capillary isoelectric focusing (icIEF).

Embodiment A30. The formulation of any one of embodiments A1-A21, wherein the anti-IL-1β antibody comprises ≥95% monomeric antibody and ≤5% is aggregated antibody after storage at 2° C. to 8° C. for six months, nine months, twelve months, eighteen months, twenty-four months, or thirty-six months.

Embodiment A31. The formulation of any one of embodiments A1-A21, wherein the anti-IL-1β antibody comprises ≥97% monomeric antibody and ≤3% is aggregated antibody after storage at 2° C. to 8° C. for six months, nine months, twelve months, eighteen months, twenty-four months, or thirty-six months.

Embodiment A32. The formulation of any one of embodiments A1-A21, wherein the anti-IL-1β antibody comprises ≥98% monomeric antibody and ≤2% is aggregated antibody after storage at 2° C. to 8° C. for six months, nine months, twelve months, eighteen months, twenty-four months, or thirty-six months.

Embodiment A33. The formulation of any one of embodiments A1-A21, wherein the anti-IL-1β antibody comprises ≥99% monomeric antibody and ≤1% is aggregated antibody after storage at 2° C. to 8° C. for six months, nine months, twelve months, eighteen months, twenty-four months, or thirty-six months.

Embodiment A34. The formulation of any one of embodiments A1-A21, wherein the anti-IL-1β antibody comprises ≤0.2% fragments after storage at 2° C. to 8° C. for six months, nine months, twelve months, eighteen months, twenty-four months, or thirty-six months.

Embodiment A35. The formulation of any one of embodiments A1-A21, wherein the anti-IL-1β antibody comprises ≤0.1% fragments after storage at 2° C. to 8° C. for six months, nine months, twelve months, eighteen months, twenty-four months, or thirty-six months.

Embodiment A36. The formulation of any one of embodiments A1-A21, wherein ≥50% of the anti-IL-1β antibodies in the formulation are the main isoform as determined by imaged capillary isoelectric focusing (icIEF) after storage at 2° C. to 8° C. for six months, nine months, twelve months, eighteen months, twenty-four months, or thirty-six months.

Embodiment A37. The formulation of any one of embodiments A1-A21, wherein ≥60% of the anti-IL-1β antibodies in the formulation are the main isoform as determined by imaged capillary isoelectric focusing (icIEF) after storage at 2° C. to 8° C. for six months, nine months, twelve months, eighteen months, twenty-four months, or thirty-six months.

Embodiment A38. The formulation of any one of embodiments A1-A21, wherein the anti-IL-1β antibody comprises ≥95% monomeric antibody and ≤5% is aggregated antibody after storage at ≥−65° C. for six months, nine months, twelve months, eighteen months, twenty-four months, or thirty-six months.

Embodiment A39. The formulation of any one of embodiments A1-A21, wherein the anti-IL-1β antibody comprises ≥97% monomeric antibody and ≤3% is aggregated antibody after storage at ≥−65° C. for six months, nine months, twelve months, eighteen months, twenty-four months, or thirty-six months.

Embodiment A40. The formulation of any one of embodiments A1-A21, wherein the anti-IL-1β antibody comprises ≥98% monomeric antibody and ≤2% is aggregated antibody after storage at ≥−65° C. for six months, nine months, twelve months, eighteen months, twenty-four months, or thirty-six months.

Embodiment A41. The formulation of any one of embodiments A1-A21, wherein the anti-IL-1β antibody comprises ≥99% monomeric antibody and ≤1% is aggregated antibody after storage at ≥−65° C. for six months, nine months, twelve months, eighteen months, twenty-four months, or thirty-six months.

Embodiment A42. The formulation of any one of embodiments A1-A21, wherein the anti-IL-1β antibody comprises ≤0.2% fragments after storage at ≥−65° C. for six months, nine months, twelve months, eighteen months, twenty-four months, or thirty-six months.

Embodiment A43. The formulation of any one of embodiments A1-A21, wherein the anti-IL-1β antibody comprises ≤0.1% fragments after storage at ≥−65° C. for six months, nine months, twelve months, eighteen months, twenty-four months, or thirty-six months.

Embodiment A44. The formulation of any one of embodiments A1-A21, wherein ≥50% of the anti-IL-1β antibodies in the formulation are the main isoform as determined by imaged capillary isoelectric focusing (icIEF) after storage at ≥−65° C. for six months, nine months, twelve months, eighteen months, twenty-four months, or thirty-six months.

Embodiment A45. The formulation of any one of embodiments A1-A21, wherein ≥60% of the anti-IL-1β antibodies in the formulation are the main isoform as determined by imaged capillary isoelectric focusing (icIEF) after storage at ≥−65° C. for six months, nine months, twelve months, eighteen months, twenty-four months, or thirty-six months.

Embodiment A46. The formulation of any one of embodiments A1-A21, wherein the anti-IL-1β antibody comprises ≥95% monomeric antibody and ≤5% is aggregated antibody after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A47. The formulation of any one of embodiments A1-A21, wherein the anti-IL-1β antibody comprises ≥97% monomeric antibody and ≤3% is aggregated antibody after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A48. The formulation of any one of embodiments A1-A21, wherein the anti-IL-1β antibody comprises ≥98% monomeric antibody and ≤2% is aggregated antibody after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A49. The formulation of any one of embodiments A1-A21, wherein the anti-IL-1β antibody comprises ≥99% monomeric antibody and ≤1% is aggregated antibody after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A50. The formulation of any one of embodiments A1-A21, wherein the anti-IL-1β antibody comprises ≤0.2% fragments after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A51. The formulation of any one of embodiments A1-A21, wherein the anti-IL-1β antibody comprises ≤0.1% fragments after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A52. The formulation of any one of embodiments A1-A21, wherein ≥50% of the anti-IL-1β antibodies in the formulation are the main isoform as determined by imaged capillary isoelectric focusing (icIEF) after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

Embodiment A53. The formulation of any one of embodiments A1-A21, wherein ≥60% of the anti-IL-1β antibodies in the formulation are the main isoform as determined by imaged capillary isoelectric focusing (icIEF) after storage at ≥−65° C. for six months, nine months, twelve months, eighteen months, twenty-four months, or thirty-six months followed by storage at 2° C. to 8° C. for six months, nine months, twelve months, eighteen months, twenty-four months, or thirty-six months.

### Sequences

The following Table 21 provides the sequences referred to in this application.

| SEQ ID NO: | Description | Sequence |
|---|---|---|
| 1 | VH CDR1 | Gly Tyr Thr Phe Asp Arg Tyr |
| 2 | VH CDR2 | Leu Pro Gly Ser Gly Asp |
| 3 | VH CDR3 | Met Tyr Tyr Asp Tyr Asp Gln Gly Phe Asp Leu |
| 4 | VL CDR1 | Lys Phe Ser Gln Asp Ile Asp Arg Phe Leu Ser |
| 5 | VL CDR2 | Arg Val Lys Arg Leu Val Asp |
| 6 | VL CDR3 | Val Gln Tyr Asp Glu Phe Pro Tyr Gly |
| 7 | Heavy chain | Gln Val Gln Leu Val Gln Ser Gly Ala Glu Val Lys Lys Pro |
| | variable region | Gly Ser Ser Val Lys Val Ser Cys Lys Ala Ser Gly Tyr Thr |
| | (VH) | Phe Asp Arg Tyr Trp Ile Glu Trp Val Arg Gln Ala Pro Gly |
| | | Gln Gly Leu Glu Trp Met Gly Glu Ile Leu Pro Gly Ser Gly |
| | | Asp Ile Asn Tyr Asn Glu Lys Phe Lys Gly Arg Val Thr Ile |
| | | Thr Ala Asp Lys Ser Thr Ser Thr Ala Tyr Met Glu Leu Ser |
| | | Ser Leu Arg Ser Glu Asp Thr Ala Val Tyr Tyr Cys Ala Arg |
| | | Met Tyr Tyr Asp Tyr Asp Gln Gly Phe Asp Leu Trp Gly |
| | | Gln Gly Thr Leu Val Thr Val Ser Ser |
| 8 | Light chain | Asp Ile Gln Met Thr Gln Ser Pro Ser Ser Leu Ser Ala Ser |
| | variable region | Val Gly Asp Arg Val Thr Ile Thr Cys Lys Phe Ser Gln Asp |
| | (VL) | Ile Asp Arg Phe Leu Ser Trp Phe Gln Gln Lys Pro Gly Lys |
| | | Ala Pro Lys Ser Leu Ile Tyr Arg Val Lys Arg Leu Val Asp |
| | | Gly Val Pro Ser Arg Phe Ser Gly Ser Gly Ser Gly Thr Asp |
| | | Phe Thr Leu Thr Ile Ser Ser Leu Gln Pro Glu Asp Phe Ala |
| | | Thr Tyr Tyr Cys Val Gln Tyr Asp Glu Phe Pro Tyr Gly Phe |
| | | Gly Gly Gly Thr Lys Val Glu Ile Lys |
| 9 | Heavy chain | Met Glu Thr Asp Thr Leu Leu Leu Trp Val Leu Leu Leu |
| | (with leader | Trp Val Pro Gly Ser Thr Gly Gln Val Gln Leu Val Gln Ser |
| | sequence) | Gly Ala Glu Val Lys Lys Pro Gly Ser Ser Val Lys Val Ser |
| | | Cys Lys Ala Ser Gly Tyr Thr Phe Asp Arg Tyr Trp Ile Glu |
| | | Trp Val Arg Gln Ala Pro Gly Gln Gly Leu Glu Trp Met Gly |
| | | Glu Ile Leu Pro Gly Ser Gly Asp Ile Asn Tyr Asn Glu Lys |
| | | Phe Lys Gly Arg Val Thr Ile Thr Ala Asp Lys Ser Thr Ser |
| | | Thr Ala Tyr Met Glu Leu Ser Ser Leu Arg Ser Glu Asp Thr |
| | | Ala Val Tyr Tyr Cys Ala Arg Met Tyr Tyr Asp Tyr Asp |
| | | Gln Gly Phe Asp Leu Trp Gly Gln Gly Thr Leu Val Thr |
| | | Val Ser Ser Ala Ser Thr Lys Gly Pro Ser Val Phe Pro Leu |
| | | Ala Pro Cys Ser Arg Ser Thr Ser Glu Ser Thr Ala Ala Leu |
| | | Gly Cys Leu Val Lys Asp Tyr Phe Pro Glu Pro Val Thr Val |
| | | Ser Trp Asn Ser Gly Ala Leu Thr Ser Gly Val His Thr Phe |
| | | Pro Ala Val Leu Gln Ser Ser Gly Leu Tyr Ser Leu Ser Ser |
| | | Val Val Thr Val Pro Ser Ser Ser Leu Gly Thr Lys Thr Tyr |
| | | Thr Cys Asn Val Asp His Lys Pro Ser Asn Thr Lys Val Asp |
| | | Lys Arg Val Glu Ser Lys Tyr Gly Pro Pro Cys Pro Pro Cys |
| | | Pro Ala Pro Glu Phe Leu Gly Gly Pro Ser Val Phe Leu Phe |
| | | Pro Pro Lys Pro Lys Asp Thr Leu Met Ile Ser Arg Thr Pro |
| | | Glu Val Thr Cys Val Val Val Asp Val Ser Gln Glu Asp Pro |
| | | Glu Val Gln Phe Asn Trp Tyr Val Asp Gly Val Glu Val His |
| | | Asn Ala Lys Thr Lys Pro Arg Glu Glu Gln Phe Asn Ser Thr |
| | | Tyr Arg Val Val Ser Val Leu Thr Val Leu His Gln Asp Trp |
| | | Leu Asn Gly Lys Glu Tyr Lys Cys Lys Val Ser Asn Lys |
| | | Gly Leu Pro Ser Ser Ile Glu Lys Thr Ile Ser Lys Ala Lys |
| | | Gly Gln Pro Arg Glu Pro Gln Val Tyr Thr Leu Pro Pro Ser |
| | | Gln Glu Glu Met Thr Lys Asn Gln Val Ser Leu Thr Cys |
| | | Leu Val Lys Gly Phe Tyr Pro Ser Asp Ile Ala Val Glu Trp |
| | | Glu Ser Asn Gly Gln Pro Glu Asn Asn Tyr Lys Thr Thr Pro |
| | | Pro Val Leu Asp Ser Asp Gly Ser Phe Phe Leu Tyr Ser Arg |
| | | Leu Thr Val Asp Lys Ser Arg Trp Gln Glu Gly Asn Val |
| | | Phe Ser Cys Ser Val Met His Glu Ala Leu His Asn His Tyr |
| | | Thr Gln Lys Ser Leu Ser Leu Ser Leu Gly Lys |
| 10 | Heavy chain | Gln Val Gln Leu Val Gln Ser Gly Ala Glu Val Lys Lys Pro |
| | (without leader | Gly Ser Ser Val Lys Val Ser Cys Lys Ala Ser Gly Tyr Thr |
| | sequence) | Phe Asp Arg Tyr Trp Ile Glu Trp Val Arg Gln Ala Pro Gly |
| | | Gln Gly Leu Glu Trp Met Gly Glu Ile Leu Pro Gly Ser Gly |
| | | Asp Ile Asn Tyr Asn Glu Lys Phe Lys Gly Arg Val Thr Ile |
| | | Thr Ala Asp Lys Ser Thr Ser Thr Ala Tyr Met Glu Leu Ser |
| | | Ser Leu Arg Ser Glu Asp Thr Ala Val Tyr Tyr Cys Ala Arg |
| | | Met Tyr Tyr Asp Tyr Asp Gln Gly Phe Asp Leu Trp Gly |
| | | Gln Gly Thr Leu Val Thr Val Ser Ser Ala Ser Thr Lys Gly |
| | | Pro Ser Val Phe Pro Leu Ala Pro Cys Ser Arg Ser Thr Ser |
| | | Glu Ser Thr Ala Ala Leu Gly Cys Leu Val Lys Asp Tyr Phe |
| | | Pro Glu Pro Val Thr Val Ser Trp Asn Ser Gly Ala Leu Thr |
| | | Ser Gly Val His Thr Phe Pro Ala Val Leu Gln Ser Ser Gly |
| | | Leu Tyr Ser Leu Ser Ser Val Val Thr Val Pro Ser Ser Ser |
| | | Leu Gly Thr Lys Thr Tyr Thr Cys Asn Val Asp His Lys Pro |
| | | Ser Asn Thr Lys Val Asp Lys Arg Val Glu Ser Lys Tyr Gly |
| | | Pro Pro Cys Pro Pro Cys Pro Ala Pro Glu Phe Leu Gly Gly |
| | | Pro Ser Val Phe Leu Phe Pro Pro Lys Pro Lys Asp Thr Leu |
| | | Met Ile Ser Arg Thr Pro Glu Val Thr Cys Val Val Val Asp |
| | | Val Ser Gln Glu Asp Pro Glu Val Gln Phe Asn Trp Tyr Val |
| | | Asp Gly Val Glu Val His Asn Ala Lys Thr Lys Pro Arg Glu |
| | | Glu Gln Phe Asn Ser Thr Tyr Arg Val Val Ser Val Leu Thr |
| | | Val Leu His Gln Asp Trp Leu Asn Gly Lys Glu Tyr Lys |
| | | Cys Lys Val Ser Asn Lys Gly Leu Pro Ser Ser Ile Glu Lys |
| | | Thr Ile Ser Lys Ala Lys Gly Gln Pro Arg Glu Pro Gln Val |
| | | Tyr Thr Leu Pro Pro Ser Gln Glu Glu Met Thr Lys Asn Gln |
| | | Val Ser Leu Thr Cys Leu Val Lys Gly Phe Tyr Pro Ser Asp |
| | | Ile Ala Val Glu Trp Glu Ser Asn Gly Gln Pro Glu Asn Asn |
| | | Tyr Lys Thr Thr Pro Pro Val Leu Asp Ser Asp Gly Ser Phe |
| | | Phe Leu Tyr Ser Arg Leu Thr Val Asp Lys Ser Arg Trp Gln |
| | | Glu Gly Asn Val Phe Ser Cys Ser Val Met His Glu Ala Leu |
| | | His Asn His Tyr Thr Gln Lys Ser Leu Ser Leu Ser Leu Gly |
| | | Lys |
| 11 | Light chain | Asp Ile Gln Met Thr Gln Ser Pro Ser Ser Leu Ser Ala Ser |
| | | Val Gly Asp Arg Val Thr Ile Thr Cys Lys Phe Ser Gln Asp |
| | | Ile Asp Arg Phe Leu Ser Trp Phe Gln Gln Lys Pro Gly Lys |
| | | Ala Pro Lys Ser Leu Ile Tyr Arg Val Lys Arg Leu Val Asp |
| | | Gly Val Pro Ser Arg Phe Ser Gly Ser Gly Ser Gly Thr Asp |
| | | Phe Thr Leu Thr Ile Ser Ser Leu Gln Pro Glu Asp Phe Ala |
| | | Thr Tyr Tyr Cys Val Gln Tyr Asp Glu Phe Pro Tyr Gly Phe |
| | | Gly Gly Gly Thr Lys Val Glu Ile Lys Arg Thr Val Ala Ala |
| | | Pro Ser Val Phe Ile Phe Pro Pro Ser Asp Glu Gln Leu Lys |
| | | Ser Gly Thr Ala Ser Val Val Cys Leu Leu Asn Asn Phe Tyr |
| | | Pro Arg Glu Ala Lys Val Gln Trp Lys Val Asp Asn Ala |
| | | Leu Gln Ser Gly Asn Ser Gln Glu Ser Val Thr Glu Gln Asp |
| | | Ser Lys Asp Ser Thr Tyr Ser Leu Ser Ser Thr Leu Thr Leu |
| | | Ser Lys Ala Asp Tyr Glu Lys His Lys Val Tyr Ala Cys Glu |
| | | Val Thr His Gln Gly Leu Ser Ser Pro Val Thr Lys Ser Phe |
| | | Asn Arg Gly Glu Cys |
| 12 | Alternative | Asp Ile Gln Met Thr Gln Ser Pro Ser Ser Leu Ser Ala Ser |
| | Light chain | Val Gly Asp Arg Val Thr Ile Thr Cys Lys Phe Ser Gln Asp |
| | variable region | Ile Asp Arg Phe Leu Ser Trp Phe Gln Gln Lys Pro Gly Lys |
| | (VL) | Ala Pro Lys Ser Leu Ile Tyr Arg Val Lys Arg Leu Val Asp |
| | | Gly Val Pro Ser Arg Phe Ser Gly Ser Gly Ser Gly Thr Asp |
| | | Phe Thr Leu Thr Ile Ser Ser Leu Gln Pro Glu Asp Phe Ala |
| | | Thr Tyr Tyr Cys Val Gln Tyr Asp Glu Phe Pro Tyr Gly Phe |
| | | Gly Gly |

1. A liquid pharmaceutical formulation comprising: a concentration of an anti-IL-1β antibody of 100 mg/mL to 200 mg/mL; at least one buffering agent; at least one colloidal stabilizer; at least one antioxidant; at least one saccharide; and at least one surfactant, wherein the anti-IL-1β antibody comprises:
(i) a heavy chain CDR1 (HCDR1) comprising the amino acid sequence of SEQ ID NO: 1; a heavy chain CDR2 (HCDR2) comprising the amino acid sequence of SEQ ID NO: 2; a heavy chain CDR3 (HCDR3) comprising the amino acid sequence of SEQ ID NO: 3; a light chain CDR1 (LCDR1) comprising the amino acid sequence of SEQ ID NO: 4; a light chain CDR2 (LCDR2) comprising the amino acid sequence of SEQ ID NO: 5; and a light chain CDR3 (LCDR3) comprising the amino acid sequence of SEQ ID NO: 6; or
(ii) a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6;
a VH comprising the acid sequence of SEQ ID NO: 7; a VL comprising an acid sequence chosen from: SEQ ID NO: 8 and SEQ ID NO: 12; a HC comprising an amino acid sequence chosen from: SEQ ID NO: 9 and SEQ ID NO: 10; and a LC comprising the amino acid sequence of SEQ ID NO: 11; or
(iii) variable heavy chain region (VH) comprising the amino acid sequence of SEQ ID NO: 7; or
(iv) a variable light chain region (VL) comprising an amino acid sequence chosen from: SEQ ID NO: 8 and SEQ ID NO: 12; or
(v) a VH comprising the amino acid sequence of SEQ ID NO: 7 and a VL comprising an amino acid sequence chosen from: SEQ ID NO: 8 and SEQ ID NO: 12; or
(vi) a heavy chain (HC) comprising an amino acid sequence having at least 85%, 90%, 91%, 92%, 93%, 94%, 95%, 96%, 97%, 98%, 99%, or 100% sequence identity to an amino acid sequence chosen from: SEQ ID NO: 9 and SEQ ID NO: 10; or
(vii) a light chain (LC) comprising the amino acid sequence having at least 85%, 90%, 91%, 92%, 93%, 94%, 95%, 96%, 97%, 98%, 99%, or 100% sequence identity to the amino acid sequence of SEQ ID NO: 11; or
(viii) a HC comprising an amino acid sequence having at least 85%, 90%, 91%, 92%, 93%, 94%, 95%, 96%, 97%, 98%, 99%, or 100% sequence identity to an amino acid sequence chosen from: SEQ ID NO: 9 and SEQ ID NO: 10 and a LC comprising an amino acid sequence having at least 85%, 90%, 91%, 92%, 93%, 94%, 95%, 96%, 97%, 98%, 99%, or 100% sequence identity to the amino acid sequence of SEQ ID NO: 11; or
(ix) a HC comprising an amino acid sequence chosen from: SEQ ID NO: 9 and SEQ ID NO: 10; or
(x) a LC comprising the amino acid sequence of SEQ ID NO: 11; or
(xi) a HC comprising an amino acid sequence chosen from: SEQ ID NO: 9 and SEQ ID NO: 10 and a LC comprising the amino acid sequence of SEQ ID NO: 11.

2. The liquid pharmaceutical formulation as claimed in claim 1, wherein the anti-IL-1β antibody is present at a concentration of 135 mg/ml to 165 mg/ml.

3. The liquid pharmaceutical formulation as claimed in claim 2, wherein the at least one buffering agent is present at a concentration of 10 mM to 30 mM, the at least one colloidal stabilizer is present at a concentration of 30 mM to 70 mM, the at least one antioxidant is present at a concentration of 1 to 20 mM, the at least one saccharide is present at a concentration of 70 to 150 mM, and the at least one surfactant is present at a concentration of 0.01% to 0.04% (w/v).

4. The liquid pharmaceutical formulation as claimed in claim 3 comprising 150 mg/mL of the anti-IL-1β antibody, 20 mM of the at least one buffering agent, 50 mM of the at least one colloidal stabilizer, 10 mM of the at least one antioxidant, 110 mM of the at least one saccharide, and 0.02% to 0.04% (w/v) of the at least one surfactant.

5. The liquid pharmaceutical formulation as claimed in claim 4, wherein the formulation has a pH of 5.7 to 7, and wherein the formulation comprises at least one of:
a. the at least one buffering agent is chosen from: histidine; histidine HCl; and sodium phosphate;
b. the at least one colloidal stabilizer is chosen from: arginine; arginine HCl; lysine; lysine HCl; and sodium chloride;
c. the at least one saccharide acts as both a conformational stabilizer and a tonicity agent and is chosen from: sucrose, glucose; fructose; galactose; mannose; arabinose; xylose; erythrose; lactose; maltose; and trehalose;
d. the at least one antioxidant is methionine;
e. the at least one surfactant is a polysorbate; and
f. the at least one surfactant is PS80.

6-7. (canceled)

8. The liquid pharmaceutical formulation as claimed in claim 7, wherein the at least one saccharide is 110 mM sucrose, wherein the sucrose raises the tonicity of the formulation to an osmolality of 243-343 mOsmo/Kg.

9-12. (canceled)

13. The liquid pharmaceutical formulation as claimed in claim 12, wherein the at least one buffering agent is histidine, the at least one colloidal stabilizer is arginine, the at least one saccharide is sucrose, the at least one antioxidant is methionine, and the at least one surfactant is PS80, and wherein the pH of the formulation ranges from 5.7 to 6.5.

14. The liquid pharmaceutical formulation as claimed in claim 13, wherein the formulation is suitable for a route of administration chosen from: intravenous infusion and subcutaneous injection.

15. A liquid pharmaceutical formulation for delivery via subcutaneous injection comprising: 150 mg/ml of anti-IL-1β antibody; 20 mM histidine (11.6 mM histidine+8.4 mM histidine HCl); 50 mM arginine; 110 mM sucrose; 10 mM methionine; and 0.02% to 0.04% (w/v) polysorbate-80, wherein the anti-IL-1β antibody comprises: a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7 and a VL comprising an acid sequence chosen from: SEQ ID NO: 8 and SEQ ID NO: 12; a HC comprising an amino acid sequence chosen from: SEQ ID NO: 9 and SEQ ID NO: 10 SEQ ID NO: 9, and a LC comprising the amino acid sequence of SEQ ID NO: 11, wherein the pH of the formulation is 6.0 to 6.4.

16. A liquid pharmaceutical formulation for delivery via subcutaneous injection comprising: 150 mg/ml of anti-IL-1β antibody; 20 mM histidine (11.6 mM histidine+8.4 mM histidine HCl); 50 mM arginine; 110 mM sucrose; 10 mM methionine; and 0.04% (w/v) polysorbate-80, wherein the anti-IL-1β antibody comprises: a HCDR1 comprising the acid sequence of SEQ ID NO: 1; a HCDR2 comprising the acid sequence of SEQ ID NO: 2; a HCDR3 comprising the acid sequence of SEQ ID NO: 3; a LCDR1 comprising the acid sequence of SEQ ID NO: 4; a LCDR2 comprising the acid sequence of SEQ ID NO: 5; and a LCDR3 comprising the acid sequence of SEQ ID NO: 6; a VH comprising the acid sequence of SEQ ID NO: 7 and a VL comprising an acid sequence chosen from: SEQ ID NO: 8 and SEQ ID NO: 12; a HC comprising an amino acid sequence chosen from: SEQ ID NO: 9 and SEQ ID NO: 10 SEQ ID NO: 9, and a LC comprising the amino acid sequence of SEQ ID NO: 11, wherein the pH of the formulation is 6.2.

17. The liquid pharmaceutical formulation as claimed in claim 16, wherein the formulation is delivered via intravenous infusion.

18. The liquid pharmaceutical formulation as claimed in claim 17, wherein the anti-IL-1β antibody comprises ≥95% monomeric antibody and ≤5% aggregated antibody.

19. The liquid pharmaceutical formulation as claimed in claim 18, wherein the anti-IL-1β antibody comprises 0.1% to 0.2% fragments.

20. The liquid pharmaceutical formulation as claimed in claim 19, wherein the anti-IL-1β antibody comprises 40.0% to 65.0% of the main isoform as determined by imaged capillary isoelectric focusing (icIEF).

21. The liquid pharmaceutical formulation as claimed in claim 20, wherein the formulation may be stored at a temperature of 2° C. to 8° C. for 48 months without substantially impacting the purity of the formulation.

22. The liquid pharmaceutical formulation as claimed in claim 20, wherein the anti-IL-1β antibody comprises ≥95% monomeric antibody and ≤5% aggregated antibody after storage at 2° C. to 8° C. for forty-eight months.

23. The liquid pharmaceutical formulation as claimed in claim 20, wherein the anti-IL-1β antibody comprises ≤0.1% fragments after storage at 2° C. to 8° C. for thirty-six months.

24. The liquid pharmaceutical formulation as claimed in claim 20, wherein the anti-IL-1β antibody comprises ≥95% monomeric antibody and ≤5% is aggregated antibody after storage at −85° C. to −65° C. for thirty-six months.

25. A liquid pharmaceutical formulation comprising:
a) a means for inhibiting IL-1β; and
b) at least one buffering agent
c) at least one colloidal stabilizer;
d) at least one antioxidant;
e) at least one saccharide; and
f) at least one surfactant.

26. The liquid pharmaceutical formulation as claimed in claim 25, wherein the means for inhibiting IL-1β is an anti-IL-1β antibody.
